(19) United States
(12) Patent Application Publication
(10) Pub. No.: US 2004/0028660 A1
(43) Pub. Date: Feb. 12, 2004

(54) Methods of using JNK or MKK inhibitors to modulate cell differentiation and to treat myeloproliferative disorders and myelodysplastic syndromes

(75) Inventors: Robert J. Hariri, Florham Park, NJ (US); David I. Stirling, Warren, NJ (US); Jerome B. Zeldis, Princeton, NJ (US)

Correspondence Address: PENNIE AND EDMONDS, 1155 AVENUE OF THE AMERICAS, NEW YORK, NY, 100362711

(73) Assignees: Anthrogenesis Corporation; Celgene Corporation

(21) Appl. No.: 10/449,248

(22) Filed: May 30, 2003

Related U.S. Application Data
(60) Provisional application No. 60/384,250, filed on May 30, 2002.
(60) Provisional application No. 60/434,833, filed on Dec. 19, 2002.

(51) Int. Cl. A61K 45/00; A61K 31/505; A61K 31/425; A61K 31/42; A61K 31/4162

(52) U.S. Cl. 424/93.7; 514/275; 514/379; 514/373; 514/410; 514/406

(57) ABSTRACT

The present invention provides methods of modulating mammalian, particularly human, stem cell and progenitor cell differentiation to regulate and control the differentiation and maturation of these cells along specific cell and tissue lineages. The methods of the invention relate to the use of certain small organic molecules to modulate the differentiation of stem cell populations along specific cell and tissue lineages, particularly embryonic-like stem cells originating from a postpartum placenta or stem cells isolated form sources such as cord blood. The invention also relates to the treatment or prevention of myelodysplastic syndrome or myeloproliferative syndrome, or symptoms thereof, comprising administration of JNK or MKK inhibitors, alone or in combination, as well as with or without the use of unconditioned cells or cells conditioned in accordance with other aspects of the invention. Finally, the invention relates to the use of such differentiated stem cells in transplantation and other medical treatments.

This application claims benefit of U.S. provisional application No. 60/384,250, filed May 30, 2002 and U.S. provisional application No. 60/434,833, filed Dec. 19, 2002, each of which is incorporated by reference herein in its entirety.

### 1. INTRODUCTION

The present invention relates to methods of modulating mammalian stem cell and progenitor cell differentiation, comprising exposing a stem or progenitor cells to compounds that inhibit c-Jun N-terminal kinase (JNK) or mitogen-activated protein kinase kinase (MKK) activity. The methods of the invention are useful for regulating or controlling the differentiation or maturation of mammalian, particularly human, stem cells along specific cell and tissue lineages. The methods of the invention relate to the use of certain small organic molecules to modulate the differentiation of stem cell populations along specific cell and tissue lineages, and in particular, to the differentiation of embryonic-like stem cells originating from a postpartum placenta or for the differentiation of stem cells isolated form sources such as cord blood. The present invention also provides methods of treating or preventing a myeloproliferative disorder (“MPD”) or a myclodysplastic syndrome (“MDS”), comprising administering to a patient in need thereof an effective amount of a JNK inhibitor or a MKK inhibitor, alone or in combination. Finally, the invention relates to the use of such differentiated stem cells in transplantation and other medical treatments.

### 2. BACKGROUND OF THE INVENTION

2.1. Stem Cells

Human stem cells are totipotential or pluripotential precursor cells capable of generating a variety of mature human cell lineages. This ability serves as the basis for the cellular differentiation and specialization necessary for organ and tissue development.

Recent success at transplanting such stem cells have provided new clinical tools to reconstitute and/or supplement bone marrow after myeloablation due to disease, exposure to toxic chemical and/or radiation. Stem cells can be employed to repopulate many, if not all, tissues and restore physiologic and anatomic functionality. The application of stem cells in tissue engineering, gene therapy delivery and cell therapeutics is also advancing rapidly.

Many different types of mammalian stem cells have been characterized, including embryonic stem cells, embryonic germ cells, adult stem cells and other committed stem cells or progenitor cells. Control or regulation of the differentiation of stem cells is sill, however, difficult. Most existing methods of modulating the differentiation of stem cells are crude and unregulatable, such that stem cells will differentiate into mixtures of cell types, rather than into one (or more) desired cell type(s), or result in low yield of the product cells.

Human stem cells have been obtained from a variety of sources. See, e.g, Caplan et al. U.S. Pat. No. 5,486,359; Körbling et al., 2002, “Hepatocytes and epithelial cells of donor origin in recipients of peripheral-blood stem cells,” N. Engl. J. Med. 346(10):738-46; Naughton et al. U.S. Pat. No. 5,962,325; and Hu et al. WO 00/73421. The drawback of existing methods of obtaining stem cells, however, is that they require harvesting of marrow or periosteal cells from a donor, from which the stem cells must be subsequently isolated; they are labor-intensive; and the yield of stem cells is very low. These references do not disclose the use of JNK or MKK inhibitors or modulators to modulate the differentiation of stem or progenitor cells. Umbilical cord blood (cord blood) is a known alternative source of hematopoietic progenitor stem cells. A major limitation of stem cell procurement from cord blood, however, has been the frequently inadequate volume of cord blood obtained, resulting in insufficient cell numbers to effectively reconstitute bone marrow after transplantation.

Methods for the ex vivo expansion of cell populations have been described. See, e.g., Emerson et al., U.S. Pat. No. 6,326,198; Kraus et al., U.S. Pat. No. 6,338,942. Modulation and differentiation using small molecules is not disclosed.

A number of biomolecules have been identified as modulating stem or progenitor cell differentiation. See Rodgers et al., U.S. Pat. No. 6,335,195 (culture of hematopoietic and mesenchymal stem cells and the induction of lineage-specific cell proliferation and differentiation by growth in the presence of angiotensinogen, angiotensin I, angiotensin II AII AT₂ type 2 receptor agonists); Nadkarni et al. 1984, Tumor 70:503-505; Melchner et al., 1985, Blood 66(6):1469-1472; Slager et al., Dev. Genet. 1993;14(3):212-24, Ray et al., 1997, J. Biol. Chem. 272(30):18702-18708); Damjanov et al., 1993, Labor. Investig. 68(2):220-232; Yan et al., 2001, Devel. Biol. 235: 422-432; Hatzopoulos et al., 1998, Development 125:1457-1468 (retinoids, such as vitamin A and retinoic acid (RA); the effect of retinoids on differentiation, however, has yet to be completely understood such that it could be used as a regulatable means of controlling differentiation of stem cells).

Folic acid analogues have been shown to effect differentiation of stem cells by killing off certain populations of stem cells (DeLoia et al., 1998, Human Reproduction 13(4): 1063-1069), and thus would not be an effective tool for regulating and propagating differentiation of large quantities of stem cells for administration to a patient.

Cytokines such as IL-1, IL-2, IL-3, IL-6, IL-7, IL-11, as well as proteins such as erythropoietin, Kit ligand, M-CSF and GM-CSF, have also been shown to direct differentiation of stem cells into specific cell types in the hematopoietic lineage (Dushnik-Levinson et al., 1995, Biol. Neonate 67:77-83). These processes, however, are not well understood and still remain too crude and imprecise to allow for a regulatable means of controlling differentiation of stem cells.

2.2. c-Jun N-Terminal Kinase (JNK)

The Jun N-terminal kinase (JNK) pathway is activated by exposure of cells to environmental stress or by treatment of cells with pro-inflammatory cytokines. Targets of the JNK pathway include the transcription factors c-jun and ATF2 (Whitmarsh & Davis, J. Mol. Med. 74:589-607, 1996). These transcription factors are members of the basic leucine zipper (bZIP) group that bind as homo- and hetero-dimeric complexes to AP-1 and AP-1-like sites in the promoters of many genes (Karin et al., Curr. Opin. Cell Biol. 9:240-246, 1997). JNK binds to the N-terminal region of c-jun and ATF-2 and phosphorylates two sites within the activation domain of each transcription factor (Hibi et al., 1993, Genes Dev. 7:2135-2148; Mohit et al., 1995, Neuron 14:67-75). Three JNK enzymes have been identified as products of distinct genes (Hibi et al, supra; Mohit et al., supra). Ten different isoforms of JNK have been identified, representing alternatively spliced forms of three different genes: JNK1, JNK2 and JNK3. JNK1 and 2 are ubiquitously expressed in human tissues, whereas JNK3 is selectively expressed in the brain, heart and testis (Dong et al., Science 270:1-4, 1998). JNK1 and 2 are expressed widely in mammalian tissues, whereas JNK3 is expressed almost exclusively in the brain. Selectivity of JNK signaling is achieved via specific interactions of JNK pathway components and by use of scaffold proteins that selectively bind multiple components of the signaling cascade.

JNKs are activated by dual phosphorylation on Thr-183 and Tyr-185. JNKK1 (also known as MKK 4) and JNKK2 (MKK7), two MAPKK level enzymes, can mediate JNK activation in cells (Lin et al., 1995, Science 268:286-289; Tournier et al., 1997, Proc. Nat. Acad. Sci. USA 94:7337-7342). JNKK2 specifically phosphorylates JNK, whereas JNKK1 can also phosphorylate and activate p38. Both JNKK1 and JNKK2 are widely expressed in mammalian tissues. JNKK1 and JNKK2 are activated by the MAPKKK enzymes, MEKK1 and 2 (Lange-Carter et al., 1993, Science 260:315-319; Yan et al., 1994, Nature 372:798-781). Both MEKK1 and MEKK2 are widely expressed in mammalian tissues.

Activation of the JNK pathway has been documented in a number of disease settings, providing the rationale for targeting this pathway for drug discovery. In addition, molecular genetic approaches have validated the pathogenic role of this pathway in several diseases. For example, autoimmune and inflammatory diseases arise from the over-activation of the immune system. Activated immune cells express many genes encoding inflammatory molecules, including cytokines, growth factors, cell surface receptors, cell adhesion molecules and degradative enzymes. Many of these genes are regulated by the JNK pathway, through activation of the transcription factors AP-1 and ATF-2, including TNFα, IL-2, E-selectin and matrix metalloproteinases such as collagenase-1 (Manning A. M. and Mercurio F. Exp. Opin Invest. Drugs 6: 555-567, 1997). Monocytes, tissue macrophages and tissue mast cells are key sources of TNFα production. The JNK pathway regulates TNFα production in bacterial lipopolysaccharide-stimulated macrophages, and in mast cells stimulated through the FceRII receptor (Swantek J. L., Cobb M. H., Geppert T. D. Mol. Cell. Biol. 17:6274-6282, 1997; Ishizuka T., Tereda N., Gerwins P., Hamelmann E., Oshiba A., Fanger G. R., Johnson G. L., and Gelfland E. W. Proc. Nat. Acad. Sci. USA 94:6358-6363, 1997). Inhibition of JNK activation effectively modulates TNFα secretion from these cells. The JNK pathway therefore regulates production of this key pro-inflammatory cytokine. Matrix metalloproteinases (MMPs) promote cartilage and bone erosion in rheumatoid arthritis, and generalized tissue destruction in other autoimmune diseases. Inducible expression of MMPs, including MMP-3 and MMP-9, type II and IV collagenases, are regulated via activation of the JNK pathway and AP-1 (Gum R., Wang H., Lengyel E., Juarez J., and Boyd D). Oncogene 14:1481-1493, 1997). In human rheumatoid synoviocytes activated with TNFα, IL-1, or Fas ligand the JNK pathway is activated (Han Z., Boyle D. L., Aupperle K. R., Bennett B., Manning A. M., Firestein G. S. J. Pharm. Exp. Therap. 291:1-7, 1999; Okamoto K., Fujisawa K., Hasunuma T., Kobata T., Sumida T., and Nishioka K. Arth & Rheum 40: 919-26, 1997). Inhibition of JNK activation results in decreased AP-1 activation and collagenase-1 expression (Han et al., supra). The JNK pathway therefore regulates MMP expression in cells involved in rheumatoid arthritis.

According to European Application No. EP 1 071 429 B 1, modification of the JNK pathway may be used to treat diabetes, insulin resistance; non-insulin dependent or Type II diabetes mellitus; prediabetic conditions; polycystic ovary syndrome (PCOS); cardiovascular diseases; coronary artery disease; hyperinsulinemia; hyperlipidemia; hyperglycemia; obesity; impaired glucose tolerance (IGT); insulin resistant non-IGT (NGT); non-diagnostic glucose tolerance; diabetic complications; fatty liver; gestational diabetes mellitus (GDM); and hypertension. International application publication no. WO 02/085396 states that disorders treatable by modulation of the JNK pathway include insulin resistance; non-insulin dependent diabetes mellitus; high blood glucose levels; elevated serum insulin; insensitivity to intravenously administered insulin; obesity; diabetes; heart disease; stroke; and cancer. These references do not, however, suggest that modulation of the JNK pathway may be used to modulate the differentiation of stem cells, or may be used to treat a myeloproliferative or myelodysplastic disorder.

2.3. Mitogen-Activated Protein Kinase (MKK)

Mitogen-activated protein kinases (MAPKs) are members of conserved signal transduction pathways that activate transcription factors, translation factors and other target molecules in response to a variety of extracellular signals. MAPKs are activated by phosphorylation at a dual phosphorylation motif having the sequence Thr-X-Tyr by mitogen-activated protein kinase kinases (MKKs). In higher eukaryotes, the physiological role of MAPK signaling has been correlated with cellular events such as proliferation, oncogenesis, development and differentiation. Accordingly, the ability to regulate signal transduction via these pathways could lead to the development of treatments and preventive therapies for human diseases associated with MAPK signaling, such as inflammatory diseases, autoimmune diseases and cancer. In mammalian cells, three parallel MAPK pathways have been described. The best characterized pathway leads to the activation of the extracellular-signal-regulated kinase (ERK).

Three MKKs capable of activating p38 in vitro have been identified. MKK3 appears to be specific for p38 (i.e., does not activate JNK or ERK), while MKK4 activates both p38 and JNK (see Derijard et al., 1995, Science 267:682-685). The third MKK, MEK6, appears to be a stronger and more specific in vivo stimulator of p38 phosphorylation (see U.S. Pat. No. 6,074,862). These proteins appear to have utility in therapeutic methods for treating conditions associated with the p38 signal transduction pathway.

2.4. Myeloproliferative and Myelodysplastic Disorders

Myeloproliferative disorders (MPDs) are generally caused by acquired clonal abnormalities of the hematopoietic stem cell and include polycythemia vera, myelofibrosis, essential thrombocytosis and chronic myeloid leukemia. C. A. Linker, Blood, in CURRENT MEDICAL DIAGNOSIS & TREATMENT 2002 535 (41^{st} ed. 2002). Myclodysplastic disorders (MDSs) are a group of acquired clonal disorders of the hematopoietic stem cell and encompass several heterogeneous syndromes, including refractory anemia with or without winged sideroblasts; refractory anemia with excess blasts; and chronic myclomonocytic leukemia. Id. at 542.

Myeloproliferative disorders (MPDs) are generally caused by acquired clonal abnormalities of the hematopoietic stem cell and include polycythemia vera, myelofibrosis, essential thrombocytosis and chronic myeloid leukemia. C. A. Linker, Blood, in CURRENT MEDICAL DIAGNOSIS & TREATMENT 2002 535 (41^{st} ed. 2002). Symptoms associated with MPD include, but are not limited to, headache, dizziness, tinnitus, blurred vision, fatigue, night sweat, low-grade fever, generalized pruritus, epistaxis, blurred vision, splenomegaly, abdominal fullness, thrombosis, increased bleeding, anemia, splenic infarction, severe bone pain, hematopoiesis in the liver, ascites, esophageal varices, liver failure, respiratory distress, and priapism.

Abnormalities associated with MPD include, but are not limited to, clonal expansion of a multipotent hematopoietic progenitor cell with the overproduction of one or more of the formed elements of the blood (e.g., elevated red blood cell count, elevated white blood cell count, and/or elevated platelet count), presence of Philadelphia chromosome or bcr-abl gene, teardrop poikilocytosis on peripheral blood smear, leukoerythroblastic blood picture, giant abnormal platelets, hypercellular bone marrow with reticular or collagen fibrosis, excessive expression of inflammatory cytokines including, but not limited to, TNF-α, IL-1, IL-2 and IL-6, excessive expression of inflammation related enzymes including, but not limited to, iNOS (inducible nitric oxide synthase) and COX-2, and marked left-shifted myeloid series with a low percentage of promyelocytes and blasts.

Myelodysplastic disorders (MDSs) are a group of acquired clonal disorders of the hematopoietic stem cell and encompass several heterogeneous syndromes, including refractory anemia with or without winged sideroblasts; refractory anemia with excess blasts; and chronic myelomonocytic leukemia. C. A. Linker, Blood, in CURRENT MEDICAL DIAGNOSIS & TREATMENT 2002 535(41^{st} ed. 2002). Types of MDS include, but are not limited to, refractory anemia (RA), RA with ringed sideroblasts (RARS), RA with excess blasts (RAEB), RAEB in transformation (RAEB-T), preleukemia and chronic myelomonocytic leukemia (CMML).

There remains a clear need for improved methods for treating or preventing an MPD or MDS, as well as methods for modulating the differentiation of a mammalian stem cell or progenitor cell.

The citation of any reference in Section 2 of this application is not an admission that the reference is prior art to this application.

### 3. SUMMARY OF THE INVENTION

The present invention provides methods of modulating the differentiation of mammalian, particularly human, stem cells or progenitor cells. In particular, the methods of the invention may be employed to regulate and control the differentiation and maturation of human stem cells along specific cell and tissue lineages. The invention encompasses the use of small molecules as agents that modulate differentiation. In one embodiment, the small molecules are preferably not polypeptides, peptides, proteins, hormones, cytokines, oligonucleotides, nucleic acids or other macromolecules. In a specific embodiment, the small molecules are those disclosed in Section 4.3, below.

The methods of the invention encompass the modulation of differentiation and/or proliferation of a stem cell or progenitor cell by contacting the cell with a c-Jun N-terminal kinase (JNK) or mitogen-activated protein kinase kinase (MKK) inhibitor. The methods of the invention also encompass the regulation of differentiation of a stem cell or progenitor cell into a specific cell lineage, including, but not limited to, a mesenchymal, hematopoietic, adipogenic, hepatogenic, neurogenic, gliogenic, chondrogenic, vasogenic, myogenic, chondrogenic, or osteogenic lineage. In a particular embodiment, the methods of the invention encompass the regulation of stem cell differentiation to a cell of a hematopoietic lineage. In another embodiment, the methods of the invention relate to modulating the differentiation of stem cells to cells of a specific hematopoietic lineage, in particular, CD34+, CD133+, and CD45+ hematopoictic lineages. Further, the invention encompasses the modulation of a committed cell to a specific cell type, e.g., mesenchymal cell, hematopoietic cell, adipocyte, hepatocyte, neuroblast, glioblast, chondrocyte, endothelial cell (EC) progenitor, myocyte, chondrocyte, or osteoblast. In specific embodiments, the invention encompasses the modulation of a committed hematopoietic progenitor cell to an erythrocyte, a thrombocyte, or a leukocyte (white blood cell) such as a neutrophil, monocyte, macrophage, eosinophil, basophil, mast cell, B-cell, T-cell, or plasma cell.

Preferably, the methods of the invention may be used to suppress specifically the generation of undesired red blood cells or erythropoietic colonies (BFU-E and CFU-E), while augmenting both the generation of leukocyte and platelet forming colonies (CFU-GM) and enhancing total colony forming unit production. The methods of the invention may be used not only to regulate the differentiation of stem cells, but may also be used to stimulate the rate of colony formation, providing significant benefits to hematopoietic stem cell transplantation by improving the speed of bone marrow engraftment and recovery of leukocyte and/or platelet production.

Any mammalian stem cell can be used in accordance with the methods of the invention, including but not limited to, stem cells isolated from cord blood, placenta and other sources. The stem cells may be isolated from any mammalian species, e.g., mouse, rat, rabbit, guinea pig, dog, cat, pig, sheep, cow, horse, monkey, etc., more preferably, a human. The stem cells may include pluripotent cells, i.e., cells that have complete differentiation versatility, that are self-renewing, and can remain dormant or quiescent within tissue. The stem cells may also include multipotent cells or committed progenitor cells. In one preferred embodiment, the invention utilizes stem cells that are viable, quiescent, pluripotent stem cells that exist within the full-term placenta and can be recovered following successful birth and placental expulsion, exsanguination and perfusion, resulting in the recovery of as many as one billion nucleated cells, which yield 50 to 100 million multipotent and pluripotent stem cells.

The invention also encompasses methods for treating a patient in need thereof with a composition comprising stem cells prepared by the methods of the present invention. Such patients include, but are not limited to, those in need of a bone marrow transplant to treat a malignant disease (e.g., patients suffering from acute lymphocytic leukemia, acute myelogenous leukemia, chronic myelogenous leukemia, chronic lymphocytic leukemia, myelodysplastic syndrome (“preleukemia”), monosomy 7 syndrome, non-Hodgkin's lymphoma, neuroblastoma, brain tumors, multiple myeloma, testicular germ cell tumors, breast cancer, lung cancer, ovarian cancer, melanoma, glioma, sarcoma or other solid tumors), those in need of a bone marrow transplant to treat a non-malignant disease (e.g., patients suffering from hematologic disorders, congenital immunodeficiences, mucopolysaccharidoses, lipidoses, osteoporosis, Langerhan's cell histiocytosis, Lesch-Nyhan syndrome or glycogen storage diseases), those undergoing chemotherapy or radiation therapy, those preparing to undergo chemotherapy or radiation therapy and those who have previously undergone chemotherapy or radiation therapy. In certain embodiments, patients receive immunosuppressant therapy prior to or concurrently with the stem cell composition.

The invention further encompasses a method of treating patients in need thereof by co-administering untreated stem cells or progenitor cells in combination with a JNK or an MKK inhibitor to induce the desired stem cell differentiation in situ.

Examples of the small molecule compounds that may be used in connection with the invention, include, but are not limited to, compounds that modulate, or preferably inhibit, JNK or MKK. In one embodiment, the inhibitor of JNK or MKK is a small organic compound capable of directly inhibiting JNK or MKK activity. In another embodiment, the inhibitor of JNK or MKK modulates another component of the JNK or MKK pathway, thus inhibiting JNK or MKK activity. In another embodiment, the compound is not a polypeptide, peptide, protein, hormone, cytokine, oligonucleotide, nucleic acid or other macromolecule. Preferably, the molecular weight of the compound is less than 1000 grams/mole. Such compounds include, but are not limited to, aminopyrimidines, imidazopyridines, pyrazolopyridines, piperazines, oxindoles, pyrazinoxindoles, epinephrine derivatives, benzazoles, heteroaryls, oximes, pyrazoles, imidazoles, sulfonyl hydrazide derivatives, indazoles, anilinopyrimidine, isothiazoloanthrones, isoxazoloanthrones, isoindolanthrones, pyrazoloanthrones and salts, solvates, isomers, clathrates, pro-drugs, hydrates, polymorphs or derivatives thereof.

In another embodiment, representative JNK and MKK inhibitory compounds of the present invention, and derivatives thereof, include, but are not limited to, compounds of the following structure (I):

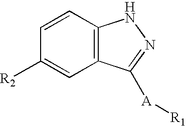

wherein A, R₁ and R₂ are as defined below (see Section 4.3), including isomers, prodrugs and pharmaceutically acceptable salts, hydrates, solvates, clathrates, polymorphs thereof.

In another embodiment, representative compounds of the present invention, and derivatives thereof, include, but are not limited to, compounds of the following structure (II):

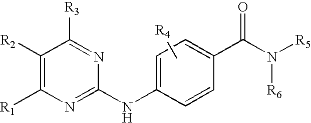

wherein R₁ though R₆ are as defined below (see Section 4.3), and including isomers, prodrugs and pharmaceutically acceptable salts, hydrates, solvates, clathrates, polymorphs thereof.

In one embodiment, representative compounds of the present invention, and derivatives thereof, include, but are not limited to, small molecules having the following structure (III):

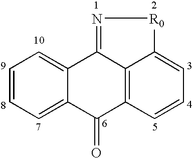

wherein R₀ is as defined below (see Section 4.3), the compound being (i) unsubstituted, (ii) monosubstituted and having a first substituent, or (iii) disubstituted and having a first substituent and a second substituent, wherein the first and second substituents are as described below, and including isomers, salts, clathrates, solvates, hydrates, prodrugs, polymorphs and pharmaceutically acceptable salts thereof.

In one particular embodiment of the invention, cells endogenous to a postpartum perfused placenta, including, but not limited to, embryonic-like stem cells, progenitor cells, pluripotent cells and multipotent cells, are exposed to the compounds of the invention and induced to differentiate. The endogenous cells may be propagated in the placenta, collected, and/or bioactive molecules recovered from the perfusate, culture medium or from the placenta cells themselves. In another embodiment, the endogenous cells may be collected from the placenta and culture medium and cultured in vitro under conditions appropriate, and for a time sufficient, to induce differentiation to the desired cell type or lineage.

In another embodiment of the invention, the stem or progenitor cells are not derived from a postpartum perfused placenta but instead, are isolated from other sources such as cord blood, bone marrow, peripheral blood or adult blood, are exposed to the compounds of the invention and induced to differentiate. In a preferred embodiment, the differentiation is conducted in vitro under conditions appropriate, and for a time sufficient, to induce differentiation into the desired lineage or cell type. The compounds of the invention are used in the differentiation/culture media by addition, in situ generation, or in any other manner that permits contact of the stem or progenitor cells with the compounds of the invention.

In sum, exposure of endogenous or exogenous stem or progenitor cells which may be cultured in a postpartum perfused placenta, to compounds of the invention may occur while the cells are cultured in the placenta, or preferably, may occur in vitro after the cells have been recovered and removed from the placenta.

The invention also encompasses the transplantation of pretreated stem or progenitor cells to treat or prevent a disease or condition. In one embodiment, the disease or condition is myelodysplastic syndrome (MDS). In another embodiment, the disease or condition is myeloproliferative disorder (MPD). In another embodiment, a patient in need of transplantation is also administered a compound of the invention before, during and/or after transplantation.

The invention further encompasses the use of a progenitor cell or specific cell type produced from a method of the invention. In other words, the invention encompasses the use of leukocytes made from the differentiation of a hematopoietic progenitor wherever said differentiation of the progenitor as modulated or regulated using a compound of the invention.

In other embodiments, the invention encompasses the control or regulation of stem cells in vivo by the administration of both a stem cell and a small molecule compound of the invention to a patient in need thereof.

In yet other embodiments, the invention encompasses methods of conditioning stem cells, comprising contacting the stem cell with a compound that modulates JNK or MKK activity for a time sufficient to effect said modulation. In a specific embodiment, said conditioning is performed following cryopreservation and thawing, to counteract the deleterious effects of cryopreservation and exposure to cryopreservatives on the stem cells. In certain embodiments, the invention provides methods of conditioning stem cells following cryopreservation and thawing, to counteract the deleterious effects of exposure to cryopreservatives (e.g., DMSO) on the proliferative and migratory capacity of stem cells. Although the invention is directed to the differentiation of human cells, the invention does not encompass the cloning of human beings or other mammals.

The invention also provides methods for the treatment of myeloproliferative disorders or myelodysplastic syndromes, comprising administering to a patient in need thereof an effective amount of a JNK inhibitor or an MKK inhibitor, or both. In certain embodiments, the myeloproliferative disorder is polycythemia rubra vera; primary thrombocythemia; chronic myelogenous leukemia; acute or chronic granulocytic leukemia; acute or chronic myclomonocytic leukemia; myelofibro-erythroleukemia; or agnogenic myeloid metaplasia.

The invention also provides a method for treating or preventing a symptom of or an abnormality associated with a myeloproliferative disorder, comprising administering to a patient in need thereof an effective amount of a JNK inhibitor or an MKK inhibitor. In a specific embodiment, the abnormality is clonal expansion of a multipotent hematopoietic progenitor cell with the overproduction of one or more of the formed elements of the blood, presence of Philadelphia chromosome or bcr-abl gene, teardrop poikilocytosis on peripheral blood smear, leukoerythroblastic blood picture, giant abnormal platelets, hypercellular bone marrow with reticular or collagen fibrosis or marked left-shifted myeloid series with a low percentage of promyclocytes and blasts.

The invention also provides a method for treating or preventing a myelodysplastic syndrome, comprising administering to a patient in need thereof an effective amount of a JNK inhibitor or an MKK inhibitor. In specific embodiments, the myelodysplastic syndrome is refractory anemia, refractory anemia with ringed sideroblasts, refractory anemia with excess blasts, refractory anemia with excess blasts in transformation, preleukemia or chronic myelomonocytic leukemia. The invention further provides a method for treating or preventing a symptom of a myclodysplastic syndrome, comprising administering to a patient in need thereof an effective amount of a JNK inhibitor or an MKK inhibitor. In specific embodiment, the symptom is anemia, thrombocytopenia, neutropenia, bicytopenia or pancytopenia.

3.1. Definitions

As used herein, the term “patient” means an animal (e.g., cow, horse, sheep, pig, chicken, turkey, quail, cat, dog, mouse, rat, rabbit or guinea pig), preferably a mammal such as a non-primate and a primate (e.g., monkey and human), most preferably a human.

“Alkyl” means a saturated straight chain or branched non-cyclic hydrocarbon having from 1 to 10 carbon atoms. “Lower alkyl” means alkyl, as defined above, having from 1 to 4 carbon atoms. Representative saturated straight chain alkyls include -methyl, -ethyl, -n-propyl, -n-butyl, -n-pentyl, -n-hexyl, -n-heptyl, -n-octyl, -n-nonyl and -n-decyl; while saturated branched alkyls include -isopropyl, -sec-butyl, -isobutyl, -tert-butyl, -isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimtheylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl and the like.

An “alkenyl group” or “alkylidene” mean a straight chain or branched non-cyclic hydrocarbon having from 2 to 10 carbon atoms and including at least one carbon-carbon double bond. Representative straight chain and branched (C₂-C₁₀)alkenyls include -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, -1-hexenyl, -2-hexenyl, -3-hexenyl, -1-heptenyl, -2-heptenyl, -3-heptenyl, -1-octenyl, -2-octenyl, -3-octenyl, -1-nonenyl, -2-nonenyl, -3-nonenyl, -1-decenyl, -2-decenyl, -3-decenyl and the like. An alkenyl group can be unsubstituted or substituted. A “cyclic alkylidene” is a ring having from 3 to 8 carbon atoms and including at least one carbon-carbon double bond, wherein the ring can have from 1 to 3 heteroatoms.

An “alkynyl group” means a straight chain or branched non-cyclic hydrocarbon having from 2 to 10 carbon atoms and including at least one carbon-carbon triple bond. Representative straight chain and branched —(C₂-C₁₀)alkynyls include -acetylenyl, -propynyl, -1-butynyl, -2-butynyl, -1-pentynyl, -2-pentynyl, -3-methyl-1-butynyl, -4-pentynyl, -1-hexynyl, -2-hexynyl, -5-hexynyl, -1-heptynyl, -2-heptynyl, -6-heptynyl, -1-octynyl, -2-octynyl, -7-octynyl, -1-nonynyl, -2-nonynyl, -8-nonynyl, -1-decynyl, -2-decynyl, -9-decynyl, and the like. An alkynyl group can be unsubstituted or substituted.

The terms “Halogen” and “Halo” mean fluorine, chlorine, bromine or iodine.

“Haloalkyl” means an alkyl group, wherein alkyl is defined above, substituted with one or more halogen atoms.

“Keto” means a carbonyl group (i.e., C═O).

“Acyl” means an —C(O)alkyl group, wherein alkyl is defined above, including —C(O)CH₃, —C(O)CH₂CH₃, —C(O)(CH₂)₂CH₃, —C(O)(CH₂)₃CH₃, —C(O)(CH₂)₄CH₃, —C(O)(CH₂)₅CH₃, and the like.

“Acyloxy” means an —OC(O)alkyl group, wherein alkyl is defined above, including —OC(O)CH₃, —OC(O)CH₂CH₃, —OC(O)(CH₂)₂CH₃, —OC(O)(CH₂)₃CH₃, —OC(O)(CH₂)₄CH₃, —OC(O)(CH₂)₅CH₃, and the like.

“Ester” and “Alkoxycarbonyl” mean a —C(O)Oalkyl group, wherein alkyl is defined above, including —C(O)OCH₃, —C(O)OCH₂CH₃, —C(O)O(CH₂)₂CH₃, —C(O)O(CH₂)₃CH₃, —C(O)O(CH₂)₄CH₃, —C(O)O(CH₂)₅CH₃, and the like.

“Alkoxy” means —O-(alkyl), wherein alkyl is defined above, including —OCH₃, —OCH₂CH₃, —O(CH₂)₂CH₃, —O(CH₂)₃CH₃, —O(CH₂)₄CH₃, —O(CH₂)₅CH₃, and the like.

“Lower alkoxy” means —O-(lower alkyl), wherein lower alkyl is as described above.

“Alkoxyalkoxy” means —O-(alkyl)-O-(alkyl), wherein each alkyl is independently an alkyl group as defined above, including —OCH₂OCH₃, —OCH₂CH₂OCH₃, —OCH₂CH₂OCH₂CH₃, and the like.

“Alkoxycarbonylalkyl” means -(alkyl)-C(═O)O-(alkyl), wherein each alkyl is independently defined above, including —CH₂—C(═O)O—CH₃, —CH₂—C(═O)O—CH₂CH₃, —CH₂—C(═O)O—(CH₂)₂CH₃, —CH₂—C(═O)O—(CH₂)₃CH₃, —CH₂—C(═O)O—(CH₂)₄CH₃, —CH₂—C(═O)O—(CH₂)₅CH₃, and the like.

“Alkoxyalkyl” means -(alkyl)-O-(alkyl), wherein each alkyl is independently an alkyl group as defined above, including —CH₂OCH₃, —CH₂OCH₂CH₃, —(CH₂)₂OCH₂CH₃, —(CH₂)₂O(CH₂)₂CH₃, and the like.

“Aryl” means a carbocyclic aromatic group containing from 5 to 10 ring atoms. Representative examples include, but are not limited to, phenyl, tolyl, anthracenyl, fluorenyl, indenyl, azulenyl, pyridinyl and naphthyl, as well as benzo-fused carbocyclic moieties including 5,6,7,8-tetrahydronaphthyl. A carbocyclic aromatic group can be unsubstituted or substituted. In one embodiment, the carbocyclic aromatic group is a phenyl group.

“Aryloxy” means —O-aryl group, wherein aryl is as defined above. An aryloxy group can be unsubstituted or substituted. In one embodiment, the aryl ring of an aryloxy group is a phenyl group

“Arylalkyl” means -(alkyl)-(aryl), wherein alkyl and aryl are as defined above, including —(CH₂)phenyl, —(CH₂)₂phenyl, —(CH₂)₃phenyl, —CH(phenyl)₂, —CH(phenyl)₃, —(CH₂)tolyl, —(CH₂)anthracenyl, —(CH₂)fluorenyl, —(CH₂)indenyl, —(CH₂)azulenyl, —(CH₂)pyridinyl, —(CH₂)naphthyl, and the like.

“Arylalkyloxy” means —O-(alkyl)-(aryl), wherein alkyl and aryl are defined above, including —O—(CH₂)₂phenyl, —O—(CH₂)₃phenyl, —O—CH(phenyl)₂, —O—CH(phenyl)₃, —O—(CH₂)tolyl, —O—(CH₂)anthracenyl, —O—(CH₂)fluorenyl, —O—(CH₂)indenyl, —O—(CH₂)azulenyl, —O—(CH₂)pyridinyl, —O—(CH₂)naphthyl, and the like.

“Aryloxyalkyl” means -(alkyl)-O-(aryl), wherein alkyl and aryl are defined above, including —CH₂—O-(phenyl), —(CH₂)₂—O-phenyl, —(CH₂)₃—O-phenyl, —(CH₂)—O-tolyl, —(CH₂)—O-anthracenyl, —(CH₂)—O-fluorenyl, —(CH₂)—O-indenyl, —(CH₂)—O-azulenyl, —(CH₂)—O-pyridinyl, —(CH₂)—O-naphthyl, and the like.

“Cycloalkyl” means a monocyclic or polycyclic saturated ring having carbon and hydrogen atoms and having no carbon-carbon multiple bonds. Examples of cycloalkyl groups include, but are not limited to, (C₃-C₇)cycloalkyl groups, including cyclopropyl, cyclobutyl, cyclopentyl, cyclohexyl, and cycloheptyl, and saturated cyclic and bicyclic terpenes. A cycloalkyl group can be unsubstituted or substituted. In one embodiment, the cycloalkyl group is a monocyclic ring or bicyclic ring.

“Cycloalkyloxy” means —O-(cycloalkyl), wherein cycloalkyl is defined above, including —O-cyclopropyl, —O-cyclobutyl, —O-cyclopentyl, —O-cyclohexyl, —O-cycloheptyl and the like.

“Cycloalkylalkyloxy” means —O-(alkyl)-(cycloalkyl), wherein cycloalkyl and alkyl are defined above, including —O—CH₂-cyclopropyl, —O—(CH₂)₂-cyclopropyl, —O—(CH₂)₃-cyclopropyl, —O—(CH₂)₄-cyclopropyl, O—CH₂-cyclobutyl, O—CH₂-cyclopentyl, O—CH₂-cyclohexyl, O—CH₂-cycloheptyl, and the like.

“Aminoalkoxy” means —O-(alkyl)-NH₂, wherein alkyl is defined above, such as —O—CH₂—NH₂, —O—(CH₂)₂—NH₂, —O—(CH₂)₃—NH₂, —O—(CH₂)₄—NH₂, —O—(CH₂)₅—NH₂, and the like.

“Mono-alkylamino” means —NH(alkyl), wherein alkyl is defined above, such as —NHCH₃, —NHCH₂CH₃, —NH(CH₂)₂CH₃, —NH(CH₂)₃CH₃, —NH(CH₂)₄CH₃, —NH(CH₂)₅CH₃, and the like.

“Di-alkylamino” means —N(alkyl)(alkyl), wherein each alkyl is independently an alkyl group as defined above, including —N(CH₃)₂, —N(CH₂CH₃)₂, —N((CH₂)₂CH₃)₂, —N(CH₃)(CH₂CH₃), and the like.

“Mono-alkylaminoalkoxy” means —O-(alkyl)-NH(alkyl), wherein each alkyl is independently an alkyl group as defined above, including —O—(CH₂)—NHCH₃, —O—(CH₂)—NHCH₂CH₃, —O—(CH₂)—NH(CH₂)₂CH₃, —O—(CH₂)—NH(CH₂)₃CH₃, —O—(CH₂)—NH(CH₂)₄CH₃, —O—(CH₂)—NH(CH₂)₅CH₃, —O—(CH₂)₂—NHCH₃, and the like.

“Di-alkylaminoalkoxy” means —O-(alkyl)-N(alkyl)(alkyl), wherein each alkyl is independently an alkyl group as defined above, including —O—(CH₂)—N(CH₃)₂, —O—(CH₂)—N(CH₂CH₃)₂, —O—(CH₂)—N((CH₂)₂CH₃)₂, —O—(CH₂)—N(CH₃)(CH₂CH₃), and the like.

“Arylamino” means —NH(aryl), wherein aryl is defined above, including —NH(phenyl), —NH(tolyl), —NH(anthracenyl), —NH(fluorenyl), —NH(indenyl), —NH(azulenyl), —NH(pyridinyl), —NH(naphthyl), and the like.

“Arylalkylamino” means —NH-(alkyl)-(aryl), wherein alkyl and aryl are defined above, including —NH—CH₂— (phenyl), —NH—CH₂— (tolyl), —NH—CH₂— (anthracenyl), —NH—CH₂-(fluorenyl), —NH—CH₂— (indenyl), —NH—CH₂— (azulenyl), —NH—CH₂— (pyridinyl), —NH—CH₂-(naphthyl), —NH—(CH₂)₂-(phenyl) and the like.

“Alkylamino” means mono-alkylamino or di-alkylamino as defined above, such as —NH(alkyl), wherein each alkyl is independently an alkyl group as defined above, including —NHCH₃, NHCH₂CH₃, NH(CH₂)₂CH₃, NH(CH₂)₃CH₃, —NH(CH₂)₄CH₃, NH(CH₂)₅CH₃, and —N(alkyl)(alkyl), wherein each alkyl is independently an alkyl group as defined above, including —N(CH₃)₂, —N(CH₂CH₃)₂, —N((CH₂)₂CH₃)₂, —N(CH₃)(CH₂CH₃) and the like.

“Cycloalkylamino” means —NH-(cycloalkyl), wherein cycloalkyl is as defined above, including —NH-cyclopropyl, —NH-cyclobutyl, —NH-cyclopentyl, —NH-cyclohexyl, —NH-cycloheptyl, and the like.

“Carboxyl” and “carboxy” mean —COOH.

“Cycloalkylalkylamino” means —NH-(alkyl)-(cycloalkyl), wherein alkyl and cycloalkyl are defined above, including —NH—CH₂-cyclopropyl, —NH—CH₂-cyclobutyl, —NH—CH₂-cyclopentyl, —NH—CH₂-cyclohexyl, —NH—CH₂-cycloheptyl, —NH—(CH₂)₂-cyclopropyl and the like.

“Aminoalkyl” means -(alkyl)-NH₂, wherein alkyl is defined above, including CH₂—NH₂, —(CH₂)₂—NH₂, —(CH₂)₃—NH₂, —(CH₂)₄—NH₂, —(CH₂)₅—NH₂: and the like.

“Mono-alkylaminoalkyl” means -(alkyl)-NH(alkyl), wherein each alkyl is independently an alkyl group defined above, including —CH₂—NH—CH₃, —CH₂—NHCH₂CH₃, —CH₂—NH(CH₂)₂CH₃, —CH₂—NH(CH₂)₃CH₃, —CH₂—NH(CH₂)₄CH₃, —CH₂—NH(CH₂)₅CH₃, —(CH₂)₂—NH—CH₃, and the like.

“Di-alkylaminoalkyl” means -(alkyl)-N(alkyl)(alkyl), wherein each alkyl is independently an alkyl group defined above, including —CH₂—N(CH₃)₂, —CH₂—N(CH₂CH₃)₂, —CH₂—N((CH₂)₂CH₃)₂, —CH₂—N(CH₃)(CH₂CH₃), —(CH₂)₂—N(CH₃)₂, and the like.

“Heteroaryl” means an aromatic heterocycle ring of 5- to 10 members and having at least one heteroatom selected from nitrogen, oxygen and sulfur, and containing at least 1 carbon atom, including both mono- and bicyclic ring systems. Representative heteroaryls are triazolyl, tetrazolyl, oxadiazolyl, pyridyl, furyl, benzofuranyl, thiophenyl, benzothiophenyl, quinolinyl, pyrrolyl, indolyl, oxazolyl, benzoxazolyl, imidazolyl, benzimidazolyl, thiazolyl, benzothiazolyl, isoxazolyl, pyrazolyl, isothiazolyl, pyridazinyl, pyrimidinyl, pyrazinyl, triazinyl, cinnolinyl, phthalazinyl, quinazolinyl, pyrimidyl, oxetanyl, azepinyl, piperazinyl, morpholinyl, dioxanyl, thietanyl and oxazolyl.

“Heteroarylalkyl” means -(alkyl)-(heteroaryl), wherein alkyl and heteroaryl are defined above, including —CH₂-triazolyl, —CH₂-tetrazolyl, —CH₂-oxadiazolyl, —CH₂-pyridyl, —CH₂-furyl, —CH₂-benzofuranyl, —CH₂-thiophenyl, —CH₂-benzothiophenyl, —CH₂-quinolinyl, —CH₂-pyrrolyl, —CH₂-indolyl, —CH₂-oxazolyl, —CH₂-benzoxazolyl, —CH₂-imidazolyl, —CH₂-benzimidazolyl, —CH₂-thiazolyl, —CH₂-benzothiazolyl, —CH₂-isoxazolyl, —CH₂-pyrazolyl, —CH₂-isothiazolyl, —CH₂-pyridazinyl, —CH₂-pyrimidinyl, —CH₂-pyrazinyl, —CH₂-triazinyl, —CH₂-cinnolinyl, —CH₂-phthalazinyl, —CH₂-quinazolinyl, —CH₂-pyrimidyl, —CH₂-oxetanyl, —CH₂-azepinyl, —CH₂-piperazinyl, —CH₂-morpholinyl, —CH₂-dioxanyl, —CH₂-thietanyl, —CH₂-oxazolyl, —(CH₂)₂-triazolyl, and the like.

“Heterocycle” means a 5- to 7-membered monocyclic, or 7- to 10-membered bicyclic, heterocyclic ring which is either saturated, unsaturated, and which contains from 1 to 4 heteroatoms independently selected from nitrogen, oxygen and sulfur, and wherein the nitrogen and sulfur heteroatoms can be optionally oxidized, and the nitrogen heteroatom can be optionally quaternized, including bicyclic rings in which any of the above heterocycles are fused to a benzene ring. The heterocycle can be attached via any heteroatom or carbon atom. Heterocycles include heteroaryls as defined above. Representative heterocycles include morpholinyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

“Heterocycle fused to phenyl” means a heterocycle, wherein heterocycle is defined as above, that is attached to a phenyl ring at two adjacent carbon atoms of the phenyl ring.

“Heterocycloalkyl” means -(alkyl)-(heterocycle), wherein alkyl and heterocycle are defined above, including —CH₂-morpholinyl, —CH₂-pyrrolidinonyl, —CH₂-pyrrolidinyl, —CH₂-piperidinyl, —CH₂-hydantoinyl, —CH₂-valerolactamyl, —CH₂-oxiranyl, —CH₂-oxetanyl, —CH₂-tetrahydrofuranyl, —CH₂-tetrahydropyranyl, —CH₂-tetrahydropyridinyl, —CH₂-tetrahydroprimidinyl, —CH₂-tetrahydrothiophenyl, —CH₂-tetrahydrothiopyranyl, —CH₂-tetrahydropyrimidinyl, —CH₂-tetrahydrothiophenyl, —CH₂-tetrahydrothiopyranyl, and the like.

The term “substituted” as used herein means any of the above groups (i.e., aryl, arylalkyl, heterocycle and heterocycloalkyl) wherein at least one hydrogen atom of the moiety being substituted is replaced with a substituent. In one embodiment, each carbon atom of the group being substituted is substituted with no more that two substituents. In another embodiment, each carbon atom of the group being substituted is substituted with no more than one substituent. In the case of a keto substituent, two hydrogen atoms are replaced with an oxygen which is attached to the carbon via a double bond. Substituents include halogen, hydroxyl, alkyl, haloalkyl, mono- or di-substituted aminoalkyl, alkyloxyalkyl, aryl, arylalkyl, heterocycle, heterocycloalkyl, —NRₐR_{b}, —NRₐC(═O)R_{b}, —NRₐC(═O)NRₐR_{b}, —NRₐC(═O)OR_{b}—NRₐSO₂R_{b}, —ORₐ, —C(═O)Rₐ C(═O)ORₐ —C(═O)NRₐR_{b}, —OC(═O)Rₐ, —OC(═O)ORₐ, —OC(═O)NRₐR_{b}, —NRₐSO₂R_{b}, or a radical of the formula —Y-Z-Rₐ where Y is alkanediyl, or a direct bond, Z is —O—, —S—, —N(R_{b})—, —C(═O)—, —C(═O)O—, —OC(═O)—, —N(R_{b})C(═O)—, —C(═O)N(R_{b})— or a direct bond, wherein Rₐ and R_{b} are the same or different and independently hydrogen, amino, alkyl, haloalkyl, aryl, arylalkyl, heterocycle, or heterocylealkyl, or wherein Rₐ and R_{b} taken together with the nitrogen atom to which they are attached form a heterocycle.

“Haloalkyl” means alkyl, wherein alkyl is defined as above, having one or more hydrogen atoms replaced with halogen, wherein halogen is as defined above, including —CF₃, —CHF₂, —CH₂F, —CBr₃, —CHBr₂, —CH₂Br, —CCl₃, —CHCl₂, —CH₂Cl, —CI₃, —CHI₁₂, —CH₂I, —CH₂—CF₃, —CH₂—CHF₂, —CH₂—CH₂F, —CH₂—CBr₃, —CH₂—CHBr₂, —CH₂—CH₂Br, —CH₂—CCl₃, —CH₂—CHCl₂, —CH₂—CH₂Cl, —CH₂—CI₃, —CH₂—CHI₂, —CH₂—CH₂I, and the like.

“Hydroxyalkyl” means alkyl, wherein alkyl is as defined above, having one or more hydrogen atoms replaced with hydroxy, including —CH₂OH, —CH₂CH₂OH, —(CH₂)₂CH₂OH, —(CH₂)₃CH₂OH, —(CH₂)₄CH₂OH, —(CH₂)₅CH₂OH, —CH(OH)—CH₃, —CH₂CH(OH)CH₃, and the like.

“Hydroxy” means —OH.

“Sulfonyl” means —SO₃H.

“Sulfonylalkyl” means —SO₂— (alkyl), wherein alkyl is defined above, including —SO₂—CH₃, —SO₂—CH₂CH₃, —SO₂—(CH₂)₂CH₃, —SO₂—(CH₂)₃CH₃, —SO₂—(CH₂)₄CH₃, —SO₂—(CH₂)₅CH₃, and the like.

“Sulfinylalkyl” means —SO-(alkyl), wherein alkyl is defined above, including —SO—CH₃, —SO—CH₂CH₃, —SO—(CH₂)₂CH₃, —SO—(CH₂)₃CH₃, —SO—(CH₂)₄CH₃, —SO—(CH₂)₅CH₃, and the like.

“Sulfonamidoalkyl” means —NHSO₂— (alkyl), wherein alkyl is defined above, including —NHSO₂—CH₃, —NHSO₂—CH₂CH₃, —NHSO₂—(CH₂)₂CH₃, —NHSO₂—(CH₂)₃CH₃, —NHSO₂—(CH₂)₄CH₃, —NHSO₂—(CH₂)₅CH₃, and the like.

“Thioalkyl” means —S-(alkyl), wherein alkyl is defined above, including —S—CH₃, —S—CH₂CH₃, —S—(CH₂)₂CH₃, —S—(CH₂)₃CH₃, —S—(CH₂)₄CH₃, —S—(CH₂)₅CH₃, and the like.

An “effective amount” when used in connection with a JNK inhibitor or MKK inhibitor is an amount of the JNK or MKK inhibitor that is useful for treating or preventing MDS, MPD or for modulating stem cell or progenitor cell differentiation.

The phrase “modulation of JNK” or “by modulating JNK” means causing a discernable inhibition or activation, preferably the inhibition, of a protein known as c-Jun N-terminal kinase (JNK) and all isoforms thereof expressed by JNK 1, JNK 2, and JNK 3 genes (Hibi et al., 1993, Genes Dev. 7:2135-2148; Mohit et al., 1995, Neuron 14:67-78; Gupta et al., 1996, EMBO J. 15:2760-2770). The modulation of JNK can be achieved on the mRNA level, protein level and kinase activity level. A compound that so modulates JNK activity is referred to herein as an “JNK modulator.”

“JNK” means a protein and all isoforms thereof expressed by JNK 1, JNK 2, and JNK 3 genes (Gupta et al., 1996, EMBO J. 15:2760-2770), including but not limited to JNK1, JNK2, and JNK3 polypeptides (Hibi et al., 1993, Genes Dev. 7:2135-2148; Mohit et al., 1995, Neuron 14:67-75).

“JNK inhibitor” or “inhibitor of JNK” means a compound capable of detectably inhibiting the activity of JNK in vitro or in vivo. The JNK inhibitor can be in the form of a or a pharmaceutically acceptable salt, free base, solvate, hydrate, stereoisomer, clathrate or prodrug thereof. Such inhibitory activity can be determined by an assay or animal model well-known in the art. In one embodiment, the JNK inhibitor is a compound of structure (I)-III) or a pharmaceutically acceptable salt, free base, solvate, hydrate, stereoisomer, clathrate, polymorph or prodrug thereof (see Section 4.3). Inhibition may be either direct or indirect; preferably, inhibition is direct. In certain embodiments, inhibitors of JNK or another component of the JNK pathway, can inhibit either upstream or downstream.

“JNK pathway” means any biological molecule which has a direct or indirect effect on the activity of JNK.

The phrase “modulation of MKK” or “by modulating MKK” means discernible inhibition or activation, preferably the inhibition, of a protein known as mitogen-activated protein kinase-kinase (MKK) and all isoforms thereof expressed by an MKK gene. The modulation of MKK can be achieved on the mRNA level, protein level and kinase activity level. A compound that so modulates MKK activity is referred to herein as an “MKK modulator.”

“MKK” means a protein and all isoforms thereof expressed by an MKK gene.

“MKK inhibitor” or “inhibitor of MKK” means a compound capable of inhibiting the activity of MKK in vitro or in vivo. The MKK inhibitor can be in the form of a or a pharmaceutically acceptable salt, free base, solvate, hydrate, stereoisomer, clathrate or prodrug thereof. Such inhibitory activity can be determined by an assay or animal model well-known in the art. In one embodiment, the MKK inhibitor is a compound of structure (I)-(III) or a pharmaceutically acceptable salt, free base, solvate, hydrate, stereoisomer, clathrate, polymorph or prodrug thereof. Inhibition may be either direct or indirect; preferably, inhibition is direct. In certain embodiments, inhibitors of MKK or another component of the JNK pathway, can inhibit either upstream or downstream.

“MKK pathway” means any biological molecule which has a direct or indirect effect on the activity of MKK.

“Direct inhibition” means that the JNK or MKK inhibitor directly interacts with JNK or MKK.

“Indirect inhibition” means that the JNK or MKK inhibitor blocks, reduces or retards JNK or MKK activity by interacting with a component of the JNK or MKK pathway other than JNK or MKK.

As used herein, the term “bioreactor” refers to an ex vivo system for propagating cells, producing or expressing biological materials and growing or culturing cells tissues, organoids, viruses, proteins, polynucleotides and microorganisms.

As used herein, the term “embryonic stem cell” refers to a cell that is derived from the inner cell mass of a blastocyst (e.g., a 4- to 5-day-old human embryo) and that is pluripotent.

As used herein, the term “embryonic-like stem cell” refers to a cell that is not derived from the inner cell mass of a blastocyst. As used herein, an “embryonic-like stem cell” may also be referred to as a “placental stem cell.” An embryonic-like stem cell is preferably pluripotent. However, the stem cells which may be obtained from the placenta include embryonic-like stem cells, multipotent cells, and committed progenitor cells. According to the methods of the invention, embryonic-like stem cells derived from the placenta may be collected from the isolated placenta once it has been exsanguinated and perfused for a period of time sufficient to remove residual cells.

As used herein, the term “exsanguinated” or “exsanguination,” when used with respect to the placenta, refers to the removal and/or draining of substantially all cord blood from the placenta by any means.

As used herein, the term “perfuse” or “perfusion” refers to the act of pouring or passaging a fluid over or through an organ or tissue, preferably the passage of fluid through an organ or tissue with sufficient force or pressure to remove any residual cells, e.g., non-attached cells from the organ or tissue. As used herein, the term “perfusate” refers to the fluid collected following its passage through an organ or tissue. In a preferred embodiment, the perfusate contains one or more anticoagulants.

As used herein, the term “multipotent cell” refers to a cell that has the capacity to grow into any of subset of the mammalian body's approximately 260 cell types. Unlike a pluripotent cell, a multipotent cell does not have the capacity to form all of the cell types.

As used herein, the term “progenitor cell” refers to a cell that is committed to differentiate into a specific type of cell or to form a specific type of tissue.

As used herein, the term “stem cell” refers to a master cell that can reproduce indefinitely to form the specialized cells of tissues and organs. A stem cell is a developmentally pluripotent or multipotent cell. A stem cell can divide to produce two daughter stem cells, or one daughter stem cell and one progenitor (“transit”) cell, which then proliferates into the tissue's mature, fully formed cells.

As used herein, the term “totipotent cell” refers to a cell that is able to form a complete embryo (e.g., a blastocyst).

As used herein, the term “exposing” when used in the context of exposing a cell to a drug or vice versa includes contacting the cell with a drug or vice versa.

As used herein, the term “pharmaceutically acceptable salt(s)” refer to a salt prepared from a pharmaceutically acceptable non-toxic acid or base including an inorganic acid and base and an organic acid and base. Suitable pharmaceutically acceptable base addition salts for the compound of the present invention include, but are not limited to metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, phosphoric, sulfuric, and methanesulfonic acids. Examples of specific salts thus include hydrochloride and mesylate salts. Others are well-known in the art, see for example, REMINGTON'S PHARMACEUTICAL SCIENCES, 18^{th} eds., Mack Publishing, Easton, Pa. (1990) or REMINGTON: THE SCIENCE AND PRACTICE OF PHARMACY, 19^{th} eds., Mack Publishing, Easton, Pa. (1995).

As used herein and unless otherwise indicated, the term “polymorph” means a different crystalline arrangement of the JNK inhibitor. Polymorphs can be obtained through the use of different work-up conditions and/or solvents. In particular, polymorphs can be prepared by recrystallization of a JNK inhibitor in a particular solvent.

As used herein and unless otherwise indicated, the term “prodrug” means a derivative of a compound that can hydrolyze, oxidize, or otherwise react under biological conditions (in vitro or in vivo) to provide an active compound, particularly a compound of the invention. Examples of prodrugs include, but are not limited to, derivatives and metabolites of a compound of the invention that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Preferably, prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6^{th} ed. (Donald J. Abraham ed., 2001, Wiley) and Design and Application of Prodrugs (H. Bundgaard ed., 1985, Harwood Academic Publishers Gmfh).

As used herein and unless otherwise indicated, the term “optically pure” or “stereomerically pure” means one stereoisomer of a compound is substantially free of other stereoisomers of that compound. For example, a stereomerically pure compound having one chiral center will be substantially free of the opposite enantiomer of the compound. A stereomerically pure a compound having two chiral centers will be substantially free of other diastereomers of the compound. A typical stereomerically pure compound comprises greater than about 80% by weight of one stereoisomer of the compound and less than about 20% by weight of other stereoisomers of the compound, more preferably greater than about 90% by weight of one stereoisomer of the compound and less than about 10% by weight of the other stereoisomers of the compound, even more preferably greater than about 95% by weight of one stereoisomer of the compound and less than about 5% by weight of the other stereoisomers of the compound, and most preferably greater than about 97% by weight of one stereoisomer of the compound and less than about 3% by weight of the other stereoisomers of the compound.

### 4. DETAILED DESCRIPTION OF THE INVENTION

The invention encompasses methods of modulating the proliferation and/or differentiation of a stem cell or progenitor cell comprising contacting the cell with an effective amount of a JNK or MKK inhibitor. In one embodiment, the present invention relates to methods of contacting a stem cell or progenitor cell with an effective amount of a JNK or MKK modulator, under conditions suitable for differentiation of the stem cell or progenitor cell, resulting in a regulatable means of controlling the differentiation of a stem or progenitor cell. In a specific, preferred embodiment, the modulator is a JNK or MKK inhibitor. In another specific embodiment, the stem cell is selected from the group consisting of an embryonic stem cell, a placental stem cell, an adult stem cell, a cord blood cell, a peripheral blood cell, and a bone marrow cell. In another specific embodiment, the stem cell is a human stem cell. In another specific embodiment, the compound is an indazole, anilinopyrimidine, isothiazoloanthrone, isoxazoloanthrone, isoindolanthrone, or pyrazoloanthrone. In another specific embodiment, the contacting step is conducted in vitro. In another specific embodiment, the contacting step is conducted in vivo. In another specific embodiment, the concentration of the compound is between 0.005 μg/ml and 5 mg/ml. In another specific embodiment, the concentration of the compound is between 1 μg/ml and 2 mg/ml.

In other embodiments, the methods of the invention encompass the regulation of stem or progenitor cell differentiation in vivo, comprising delivering the compounds to a subject that is the recipient of unconditioned stem cells, followed by direct administration of the compound to the subject.

In another embodiment, the present invention relates to methods of controlling the differentiation of a stem cell or progenitor cell, comprising exposing the cell to an effective amount of a JNK or MKK inhibitor. In another embodiment, the present invention relates to methods of exposing stem cells or progenitor cells to a JNK or MKK inhibitor, resulting in a regulatable means of controlling the differentiation of a stem or progenitor cell into a specific population of progenitor cell or differentiation of progenitor cell into a specific cell type.

In another embodiment, the exposure of a stem or progenitor cell to an effective amount of a JNK or MKK inhibitor results in the regulatable differentiation and expansion of specific populations of hematopoietic cells, including CD34+ and CD38+ cells. Further, the exposure of a hematopoietic progenitor cell to an effective amount of a JNK or MKK inhibitor results in regulatable differentiation and expansion of specific cell types.

The present invention provides methods of modulating human stem cell differentiation. In particular, the present invention provides methods that employ small organic molecules that inhibit JNK or MKK activity to modulate the differentiation of stem cell or progenitor cell populations along specific cell and tissue lineages.

Further, the invention encompasses methods of producing hematopoietic cells for transplantation into mammals, comprising exposing hematopoietic progenitor cells to a JNK or MKK inhibitor or antagonist, wherein the inhibitor or antagonist is a small molecule.

Thus, in one embodiment, the invention provides a method of producing a hematopoietic cell comprising contacting a mammalian stem cell with a compound that inhibits JNK or MKK activity under conditions suitable for differentiation of the stem cell, wherein said differentiation results in the production of a hematopoietic cell. In a specific embodiment, the stem cell is selected from the group consisting of an embryonic stem cell, a placental stem cell, an adult stem cell, a cord blood cell, a peripheral blood cell, and a bone marrow cell. In another specific embodiment, the stem cell is a human stem cell. In another specific embodiment, the compound is an indazole, anilinopyrimidine, isothiazoloanthrone, isoxazoloanthrone, isoindolanthrone, or pyrazoloanthrone. In yet another specific embodiment, the contacting step is conducted in vitro. In other specific embodiment, the concentration of the compound is between 0.005 μg/ml and 5 mg/ml, or is between 1 μg/ml and 2 mg/ml. In another specific embodiment, said hematopoietic cell is a hematopoietic progenitor cell.

Examples of the small molecule compounds that may be used in connection with the invention, include, but are not limited to, compounds that inhibit JNK or MKK activity. In one embodiment, the inhibitor of JNK or MKK is a small organic compound capable of directly inhibiting JNK or MKK activity. In another embodiment, the inhibitor of JNK or MKK inhibits another component of the JNK or MKK pathway, thus inhibiting JNK or MKK activity. In one embodiment, the compound is not a polypeptide, peptide, protein, hormone, cytokine, oligonucleotide, nucleic acid, or other macromolecule. Preferably, the molecular weight of the compound is less than 1000 grams/mole. Such compounds include, but are not limited to, indazoles, anilinopyrimidine, isothiazoloanthrones, isoxazoloanthrones, isoindolanthrones, pyrazoloanthrones and salts, solvates, isomers, clathrates, pro-drugs, hydrates or derivatives thereof. Preferably, the inhibitor of JNK or MKK is one of the compounds disclosed in Section 4.3, below, or a pharmaceutically acceptable salt, free base, solvate, hydrate, stereoisomer, clathrate or prodrug thereof.

In another embodiment, the methods of the invention encompass the regulation of differentiation of a stem cell into a specific cell lineage, including, but not limited to, a mesenchymal, hematopoietic, adipogenic, hepatogenic, neurogenic, gliogenic, chondrogenic, vasogenic, myogenic, chondrogenic, or osteogenic lineage comprising incubating the progenitor or stem cell with a compound of the invention, preferably in vitro, for a sufficient period of time to result in the differentiation of the cell into a cell of a desired cell lineage. In a specific embodiment, differentiation of a stem or progenitor cell into a cell of the hematopoietic lineage is modulated. In particular, the methods of the invention may be used to modulate the generation of blood cell colony generation from CD34+, CD133+, and CD45+ hematopoietic progenitor cells in a dose-responsive manner.

Any mammalian stem cell can be used in accordance with the methods of the invention, including but not limited to, stem cells isolated from cord blood (“CB” cells), placenta and other sources. The stem cells may include pluripotent cells, i.e., cells that have complete differentiation versatility, that are self-renewing, and can remain dormant or quiescent within tissue. The stem cells may also include multipotent cells or committed progenitor cells. In one preferred embodiment, the invention utilizes stem cells that are viable, quiescent, pluripotent stem cells that exist within the full-term placenta, and which can be recovered following successful birth and placental expulsion, exsanguination and perfusion resulting in the recovery of multipotent and pluripotent stem cells.

In a particular embodiment of the invention, cells endogenous to the placenta, including, but not limited to, embryonic-like stem cells, progenitor cells, pluripotent cells and multipotent cells, are exposed to the compounds of the invention and induced to differentiate while being cultured in an isolated and perfused placenta. The endogenous cells propagated in the postpartum perfused placental may be collected, and/or bioactive molecules recovered from the perfusate, culture medium or from the placenta cells themselves. Alternatively, exogenous cells may be propagated in the post-partum placenta. The exogenous cells are contacted with the compounds of the invention and collected from the placenta at a desired time in the same manner described for endogenous placental cells. Likewise, the cells contact with the compounds of the inventions, contained in the post-partum placenta may comprise a chimera of endogenous and exogenous cells.

In another embodiment of the invention, stem or progenitor cells that are derived from sources other than postpartum placenta are exposed to the compounds of the invention and induced to differentiate while being cultured in vitro under 2 or 3 dimensional culture conditions. Thus, the invention encompasses methods for differentiating mammalian stem cells into specific progenitor cells comprising differentiating the stem cells under conditions and/or media suitable for the desired differentiation and in the presence of a compound of the invention.

Further, the invention encompasses methods for modulating or regulating the differentiation of a population of a specific progenitor cell into specific cell types comprising differentiating said progenitor cell under conditions suitable for said differentiation and in the presence of one or more compounds of the invention. Alternatively, the stem or progenitor cell can be exposed to a compound of the invention and subsequently differentiated using suitable conditions. Examples of suitable conditions include nutrient media formulations supplemented with human serum and cell culture matrices, such as MATRIGEL® supplemented with growth factors.

The invention encompasses the modulation of stem or progenitor cells in vivo, in a patient to be treated. Thus, one or more of the JNK or MKK inhibitory compounds of the invention, alone or in combination, may be administered to a patient. In various embodiments, such compounds may be administered concurrently or serially in combination with, for example, stem or progenitor cells, the differentiation of which has been modulated using one or more of the compounds of the invention; with treated stem or progenitor cells and untreated stem or progenitor cells; with cord blood; with treated stem or progenitor cells plus cord blood. The compound and any treated or untreated cells may be administered together or separately; in the latter case, the cells or the compound(s) may be administered first.

In a specific embodiment, the present invention provides methods that employ JNK or MKK inhibitors to modulate and regulate hematopoiesis in the context of pre-transplantation conditioning of hematopoictic progenitors.

The present invention also provides methods for the conditioning of stem or progenitor cells, comprising contacting the stem or progenitor cell with a JNK or MKK modulator for a time sufficient to effect detectable modulation of differentiation of the stem cell or progenitor cell. In a specific embodiment, said JNK or MKK modulator is a JNK or MKK inhibitor. In specific embodiments, said contacting may be performed immediately after the stem cells or progenitor cells are collected, or after the stem or progenitor cells have been cryopreserved and thawed. The present invention also provides methods that employ JNK or MKK modulators, such as JNK or MKK inhibitors, to regulate hematopoiesis in the context of ex vivo conditioning of hematopoietic progenitors.

The methods of the invention encompass the regulation of stem or progenitor cell differentiation in vitro, comprising incubating the stem or progenitor cells with the compound in vitro, followed by direct transplantation of the differentiated cells to a subject. Such regulation may also take place in vivo, for example, by localized delivery of one or more of the compounds of the invention alone or in conjunction with stem or progenitor cells.

In specific embodiments of the transplantation method, the stem cell is selected from the group consisting of an embryonic stem cell, a placental stem cell, an adult stem cell, a cord blood cell, a peripheral blood cell, and a bone marrow cell. In another specific embodiment, the stem cell is a human stem cell. In another specific embodiment, the compound is an indazole, anilinopyrimidine, isothiazoloanthrone, isoxazoloanthrone, isoindolanthrone, or pyrazoloanthrone. In another specific embodiment, the contacting step is conducted in vitro. In another specific embodiment, the concentration of the compound is between 0.005 μg/ml and 5 mg/ml. In another specific embodiment, the concentration of the compound is between 1 μg/ml and 2 mg/ml.

The invention also encompasses the control or regulation of stem or progenitor cells in vivo by the administration of both a stem or progenitor cell and a compound of the invention to a patient in need thereof. The invention further encompasses the transplantation of stem or progenitor cells that have been pretreated with a JNK or MKK inhibitor, wherein said transplantation is performed to treat or prevent disease. In one embodiment, the invention provides method of transplanting a mammalian stem cell or progenitor cell to a patient in need thereof comprising: (a) contacting the stem cell or progenitor cell with a compound that inhibits JNK activity to produce a treated stem cell or progenitor cell; and (b) transplanting the treated stem cell into said patient. In a specific embodiment, the treated cell is transplanted in combination with untreated cells, such as untreated stem or progenitor cells, e.g., embryonic stem cells, placental stem cells, adult stem cells, cord blood cells, adult blood cells, peripheral blood cells, or bone marrow cells. In other embodiments, a patient in need of transplantation is also administered a compound of the invention before, during and/or after transplantation. In another embodiment, the methods of the invention include the administration of the compounds to a subject that is the recipient of unconditioned stem cells or progenitor cells for the purpose of eliciting a modulatory effect on the stem cells that have already been transplanted. In any of the transplantation methods disclosed herein, the treated and/or untreated cells may be cryopreserved and thawed prior to transplantation.

In certain embodiments, the invention encompasses bone marrow transplantation comprising transplanting cord blood (or stem cells obtained from cord blood), peripheral (i.e., adult) blood (or stem cells obtained from peripheral blood), wherein said cord blood or stem cells have been pretreated with a compound of the invention. Further, the invention encompasses the use of white blood cells made from hematopoietic progenitor cells that have been differentiated in the presence of a compound of the invention. For example, white blood cells produced by differentiating hematopoietic progenitor can be used in transplantation or can be mixed with cord blood or cord blood stem cells prior to transplantation.

Thus, the invention provides a method of treating a mammalian subject in need of white blood cells comprising differentiating a stem cell or a progenitor cell under suitable conditions and in the presence of a compound that inhibits JNK or MKK activity, wherein said differentiating produces white blood cells, and administering a therapeutically effective amount of said white blood cells to said mammalian subject. In another embodiment, the invention provides a method of treating a mammalian subject in need of white blood cells comprising administering one or more compounds of the invention in conjunction with treated or untreated stem or progenitor cells. In a specific embodiment, the stem cell or progenitor cell is differentiated in vitro. In another specific embodiment, said differentiating takes place in vivo, within said patient, after administration of one or more of the compounds of the invention. In another specific embodiment, the stem cell or progenitor cell is differentiated in a postpartum perfused placenta. In another specific embodiment, the white blood cells are administered to the recipient mammalian subject in a cell preparation that is substantially free of red blood cells. In another specific embodiment, the white blood cells are administered to the recipient mammalian subject in a cell preparation that comprises cord blood cells. In another specific embodiment, the white blood cells are administered to the recipient mammalian subject in conjunction with a carrier. In another specific embodiment, the white blood cells are administered intravenously. In another specific embodiment, the white blood cells express incorporated genetic material of interest. In another specific embodiment, said mammalian subject is human. In another specific embodiment, said white blood cells are administered in conjunction with one or more JNK or MKK modulators, preferably one or more JNK or MKK inhibitors.

In other embodiments, the invention encompasses methods for treating a patient in need of bone marrow transplantation with a composition comprising stem cells that have been differentiated in the presence of a compound of the invention. Such patients include, but are not limited to, those in need of a bone marrow transplant to treat a malignant disease (e.g., patients suffering from acute lymphocytic leukemia, acute myelogenous leukemia, chronic myelogenous leukemia, chronic lymphocytic leukemia, myelodysplastic syndrome (“preleukemia”), monosomy 7 syndrome, non-Hodgkin's lymphoma, neuroblastoma, brain tumors, multiple myeloma, testicular germ cell tumors, breast cancer, lung cancer, ovarian cancer, melanoma, glioma, sarcoma or other solid tumors) and those in need of a bone marrow transplant to treat a non-malignant disease (e.g., patients suffering from hematologic disorders, congenital immunodeficiences, mucopolysaccharidoses, lipidoses, osteoporosis, Langerhan's cell histiocytosis, Lesch-Nyhan syndrome or glycogen storage diseases).

In other embodiments, the invention encompasses methods for administering stem cells that have been differentiated in the presence of a compound of the invention to patients undergoing chemotherapy or radiation therapy or patients preparing to undergo chemotherapy or radiation therapy. In certain embodiments, patients receive immunosuppressant therapy prior to or concurrently with the stem cells composition. Such immunosuppressant therapy includes, but is not limited to, the administration of one or more therapeutic agents or radiation therapy.

The invention further encompasses methods of conditioning stem cells following cryopreservation and thawing, to counteract the deleterious effects of cryopreservation and exposure to cryopreservatives on the stem cells. In certain embodiments, the invention provides methods of conditioning stem cells following cryopreservation and thawing, to counteract the deleterious effects of exposure to cryopreservatives (e.g., DMSO) on the proliferative and migratory capacity of stem cells.

4.1. Modulation of Differentiation of Stem Cells

The present invention provides methods of modulating human stem cell differentiation. In certain embodiments, the methods of the invention encompass the regulation of stem or progenitor cell differentiation in vitro, comprising incubating the stem cells with the compound in vitro, followed by direct transplantation of the differentiated cells to a subject. In other embodiments, the methods of the invention encompass the regulation of stem or progenitor cell differentiation in vivo, comprising delivering the compounds to a subject that is the recipient of unconditioned stem cells, followed by direct administration of the compound to the subject. A combination of these methods may also be used.

The embryonic-like stem cells obtained by the methods of the invention may be induced to differentiate along specific cell lineages, including, but not limited to a mesenchymal, hematopoietic, adipogenic, hepatogenic, neurogenic, gliogenic, chondrogenic, vasogenic, myogenic, chondrogenic, or osteogenic lineage. In certain embodiments, embryonic-like stem cells obtained according to the methods of the invention are induced to differentiate for use in transplantation and ex vivo treatment protocols. In certain embodiments, embryonic-like stem cells obtained by the methods of the invention are induced to differentiate into a particular cell type and genetically engineered to provide a therapeutic gene product. In a specific embodiment, embryonic-like stem cells obtained by the methods of the invention are incubated in vitro with a compound, such as a small organic molecule, that induces the cell to differentiate, followed by direct transplantation of the differentiated cells to a subject. In a preferred embodiment, the compounds that are used to control or regulate differentiation of stem cells are not polypeptides, peptides, proteins, hormones, cytokines, oligonucleotides or nucleic acids.

Stem cells that may be used in accordance with the invention include, but are not limited to, cord blood (CB) cells, placental cells, embryonic stem (ES) cells, embryonic-like stem cells, trophoblast stem cells, progenitor cells, bone marrow stem cells and multipotent, pluripotent and totipotent cells.

In particular, the methods of the invention encompass the regulation of the differentiation of stem cell populations, in addition to mesenchymal stem cells, into specific tissue lineages. For example, the methods of the invention may be employed to regulate the differentiation of a multipotent stem cell into chondrogenic, vasogenic, myogenic, and osteogenic lineage cells by promoting specific musculoskeletal regeneration and repair, neoangiogenesis, and repopulation of specific muscular tissues, such as myocardium and skeletal muscle, and revascularization of a variety of organs and tissues including, but not limited to brain, spinal cord, liver, lung, kidney and pancreas. The methods of the invention may be employed to regulate differentiation of a multipotent stem cell into cell of adipogenic, chondrogenic, osteogenic, neurogenic or hepatogenic lineage.

The agent used to modulate differentiation can be introduced into the postpartum perfused placenta to induce differentiation of the cells being cultured in the placenta. Alternatively, the agent can be used to modulate differentiation in vitro after the cells have been collected or removed from the placenta.

The methods of the invention encompass the regulation of progenitor stem cell differentiation to a cell of the hematopoietic lineage, comprising incubating the progenitor stem cells with the compound in vitro for a sufficient period of time to result in the differentiation of these cells to a hematopoietic lineage. In particular, the methods of the invention may used to modulate the generation of blood cell colony generation from CD34+, CD133+, and CD45+ hematopoietic progenitor cells in a dose-responsive manner (for discussion of dosing, see Section 4.8).

Preferably, the methods of the invention may be used to suppress specifically the generation of unwanted red blood cells or erythropoietic colonies (BFU-E and CFU-E), while augmenting both the generation of leukocyte and platelet forming colonies (CFU-GM) and enhancing total colony forming unit production. The methods of the invention may be used not only to regulate the differentiation of stem cells, but may also be used to stimulate the rate of colony formation, providing significant benefits to hematopoietic stem cell transplantation by improving the speed of bone marrow engraftment and recovery of leukocyte and/or platelet production.

In other embodiments, the methods of the invention may be used to regulate the differentiation of e.g., a neuronal precursor cell or neuroblast into a specific neuronal cell type such as a sensory neuron (e.g., a retinal cell, an olfactory cell, a mechanosensory neuron, a chemosensory neuron, etc.), a motorneuron, a cortical neuron, or an interneuron. In other embodiments, the methods of the invention may be used to regulate the differentiation of cell types including, but not limited to, cholinergic neurons, dopaminergic neurons, GABA-ergic neurons, glial cells (including oligodendrocytes, which produce myelin), and ependymal cells (which line the brain's ventricular system). In yet other embodiments, the methods of the invention may be used to regulate the differentiation of cells that are constituent of organs, including, but not limited to, purkinje cells of the heart, biliary epithelium of the liver, beta-islet cells of the pancreas, renal cortical or medullary cells, and retinal photoreceptor cells of the eye.

Assessment of the differentiation state of stem cells obtained according to the methods of the invention may be identified by the presence or absence of certain cell surface markers. Embryonic-like stem cells of the invention, for example, may be distinguished by the following cell surface markers: OCT-4 and ABC-p, or the equivalents thereof in different mammalian species. Further, the invention encompasses embryonic-like stem cells having the following markers: CD10, CD29, CD44, CD54, CD90, SH2, SH3, SH4, OCT-4 and ABC-p, or lacking the following cell surface markers: CD34, CD38, CD45, SSEA3 and SSEA4, or the equivalents thereof in different mammalian species. The presence or absence of such cell surface markers are routinely determined according to methods well known in the art, e.g. by flow cytometry, followed by washing and staining with an anti-cell surface marker antibody. For example, to determine the presence of CD34 or CD38, cells may be washed in PBS and then double-stained with anti-CD34 phycoerythrin and anti-CD38 fluorescein isothiocyanate (Becton Dickinson, Mountain View, Calif.).

In another embodiment, differentiated stem cells are identified and characterized by a colony forming unit assay, which is commonly known in the art, such as Mesen Cult™ medium (Stem Cell Technologies, Inc., Vancouver British Columbia).

Determination that a stem cell has differentiated into a particular cell type may be accomplished by methods well-known in the art, e.g., measuring changes in morphology and cell surface markers using techniques such as flow cytometry or immunocytochemistry (e.g., staining cells with tissue-specific or cell-marker specific antibodies), by examination of the morphology of cells using light or confocal microscopy, or by measuring changes in gene expression using techniques well known in the art, such as PCR and gene-expression profiling.

In certain embodiments, differentiated cells may be identified by characterizing differentially expressed genes (for example, comparing the level of expression of a plurality of genes from an undifferentiated progenitor cell(s) of interest to the level of expression of said plurality of genes in a differentiated cell derived from that type of progenitor cell). For example, nucleic acid amplification methods such as polymerase chain reaction (PCR) or transcription-based amplification methods (e.g., in vitro transcription (IVT)) may be used to profile gene expression in different populations of cells, e.g., by use of a polynucleotide microarray. Such methods to profile differential gene expression are well known in the art. See, e.g., Wieland et al., 1990, Proc. Natl. Acad. Sci. USA 87: 2720-2724; Lisitsyn et al., 1993, Science 259: 946-951; Lisitsyn et al., 1995, Meth. Enzymol. 254:291-304; U.S. Pat. No. 5,436,142; U.S. Pat. No. 5,501,964; Lisitsyn et al., 1994, Nature Genetics 6:57-63; Hubank and Schatz, 1994, Nucleic Acids Res. 22: 5640-5648; Zeng et al., 1994, Nucleic Acids Research 22: 4381-4385; U.S. Pat. No. 5,525,471; Linsley et al., U.S. Pat. No. 6,271,002, entitled “RNA amplification method,” issued Aug. 7, 2001; Van Gelder et al., U.S. Pat. No. 5,716,785, entitled “Processes for genetic manipulations using promoters,” issued Feb. 10, 1998; Stoflet et al., 1988, Science 239:491-494; Sarkar and Sommer, 1989, Science 244:331-334; Mullis et al., U.S. Pat. No. 4,683,195; Malek et al., U.S. Pat. No. 5,130,238; Kacian and Fultz, U.S. Pat. No. 5,399,491; Burg et al., U.S. Pat. No. 5,437,990; van Gelder et al., 1990, Proc. Natl. Acad. Sci. USA 87:1663; Lockhart et al., 1996, Nature Biotechnol. 14:1675; Shannon, U.S. Pat. No. 6,132,997; Lindemann et al., U.S. Pat. No. 6,235,503, entitled “Procedure for subtractive hybridization and difference analysis,” issued May 22, 2001.

Commercially available kits are available for gene profiling, e.g., the displayPROFILE™ series of kits (Qbiogene, Carlsbad, Calif.), which uses a gel-based approach for profiling gene expression. The kits utilize Restriction Fragment Differential Display-PCR(RFDD-PCR) to compare gene expression patterns in eukaryotic cells. A PCR-Select Subtraction Kit (Clontech) and a PCR-Select Differential Screening Kit (Clontech) may also be used, which permits identification of differentially expressed clones in a subtracted library. After generating pools of differentially expressed genes with the PCR-Select Subtraction kit, the PCR-Select Differential Screening kit is used. The subtracted library is hybridized with probes synthesized directly from tester and driver populations, a probe made from the subtracted cDNA, and a probe made from reverse-subtracted cDNA (a second subtraction performed in reverse). Clones that hybridize to tester but not driver probes are differentially expressed; however, non-subtracted probes are not sensitive enough to detect rare messages. Subtracted probes are greatly enriched for differentially expressed cDNAs, but may give false positive results. Using both subtracted and non-subtracted probes according to the manufacturer's (Clontech) instructions identifies differentially expressed genes.

4.2. Stem Cell Populations

The present invention provides methods of modulating human stem cell differentiation. Any mammalian stem cell can be used within the methods of the invention, including, but not limited to, stem cells isolated from cord blood (CB cells), peripheral blood, adult blood, bone marrow, placenta, mesenchymal stem cells and other sources. In a non-preferred embodiment, the stem cells are embryonic stem cells or cells that have been isolated from sources other than placenta.

Sources of mesenchymal stem cells include bone marrow, embryonic yolk sac, placenta, umbilical cord, fetal and adolescent skin, and blood. Bone marrow cells may be obtained, for example, from iliac crest, femora, tibiae, spine, rib or other medullary spaces.

The stem cells to be used in accordance with the methods of the present invention may include pluripotent cells, i.e., cells that have complete differentiation versatility, that are self-renewing, and can remain dormant or quiescent within tissue. The stem cells may also include multipotent cells, committed progenitor cells, and fibroblastoid cells. In one preferred embodiment, the invention utilizes stem cells that are viable, quiescent, pluripotent stem cells isolated from a full-term exsanguinated perfused placenta.

Stem cell populations may consist of placental stem cells obtained through a commercial service, e.g., LifeBank USA (Cedar Knolls, N.J.), ViaCord (Boston Mass.), Cord Blood Registry (San Bruno, Calif.) and Cryocell (Clearwater, Fla.). Stem and/or progenitor cells may also be collected using processes known in the art, e.g., apheresis or leukapheresis. Stem cell populations may be used in relatively unpurified form, as in cord blood or in populations of peripheral blood mononuclear cells obtained by apheresis, or relatively purified, i.e., substantially purified from other cell types.

Stem cell populations may also consist of placental stem cells collected according to the methods disclosed in co-pending U.S. application Ser. No. 10/004,942, filed Dec. 5, 2001, entitled “Method of Collecting Placental Stem Cells” and U.S. application Ser. No. 10/076,180, filed Feb. 13, 2002, entitled “Post-Partum Mammalian Placenta, Its Use and Placental Stem Cells Therefrom” (both of which are incorporated herein by reference in their entireties).

In one embodiment, stem cells from cord blood may be used. The first collection of blood from the placenta is referred to as cord blood, which contains predominantly CD34+ and CD38+ hematopoietic progenitor cells. Within the first twenty-four hours of postpartum perfusion, high concentrations of CD34+ CD38− hematopoietic progenitor cells may be isolated from the placenta. After about twenty-four hours of perfusion, high concentrations of CD34− CD38− cells can be isolated from the placenta along with the aforementioned cells. The isolated perfused placenta of the invention provides a source of large quantities of stem cells enriched for CD34+ CD38− stem cells and CD34− CD38+ stem cells. The isolated placenta that has been perfused for twenty-four hours or more provides a source of large quantities of stem cells enriched for CD34− and CD38− stem cells.

Preferred cells to be used in accordance with the present invention are embryonic-like stem cells that originate from an exsanguinated perfused placenta, or cells that derive from embryonic-like placental stem cells. The embryonic-like stem cells of the invention may be characterized by measuring changes in morphology and cell surface markers using techniques such as flow cytometry and immunocytochemistry, and measuring changes in gene expression using techniques, such as PCR. In one embodiment of the invention, such embryonic-like stem cells may be characterized by the presence or absence of the following cell surface markers: CD10, CD29, CD44, CD54, CD90, SH2, SH3, SH4, OCT-4 and ABC-p, or the absence of the following cell surface markers: CD34, CD38, CD45, SSEA3 and SSEA4, or the equivalents thereof in different mammalian species. In a preferred embodiment, such embryonic-like stem cells may be characterized by the presence of cell surface markers OCT-4 and ABC-p, or the equivalents thereof in different mammalian species. Such cell surface markers are routinely determined according to methods well known in the art, e.g. by flow cytometry, followed by washing and staining with an anti-cell surface marker antibody. For example, to determine the presence of CD34 or CD38, cells may be washed in PBS and then double-stained with anti-CD34 phycoerythrin and anti-CD38 fluorescein isothiocyanate (Becton Dickinson, Mountain View, Calif.).

Embryonic-like stem cells originating from placenta have characteristics of embryonic stem cells but are not derived from the embryo. In other words, the invention encompasses the use of OCT-4+ and ABC-p+ cells that are undifferentiated stem cells that are isolated from a postpartum perfused placenta. Such cells are as versatile (e.g., pluripotent) as human embryonic stem cells. As mentioned above, a number of different pluripotent or multipotent stem cells can be isolated from the perfused placenta at different time points e.g., CD34+ CD38+, CD34+ CD38−, and CD34−CD38− hematopoietic cells. According to the methods of the invention, human placenta is used post-birth as the source of embryonic-like stem cells.

For example, after expulsion from the womb, the placenta is exsanguinated as quickly as possible to prevent or minimize apoptosis. Subsequently, as soon as possible after exsanguination the placenta is perfused to remove blood, residual cells, proteins, factors and any other materials present in the organ. Material debris may also be removed from the placenta. Perfusion is normally continued with an appropriate perfusate for at least two to more than twenty-four hours. In several additional embodiments the placenta is perfused for at least 4, 6, 8, 10, 12, 14, 16, 18, 20, and 22 hours. In other words, this invention is based at least in part on the discovery that the cells of a postpartum placenta can be activated by exsanguination and perfusion for a sufficient amount of time. Therefore, the placenta can readily be used as a rich and abundant source of embryonic-like stem cells, which cells can be used for research, including drug discovery, treatment and prevention of diseases, in particular transplantation surgeries or therapies, and the generation of committed cells, tissues and organoids. See co-pending U.S. Application Ser.

No. 10/004,942, filed Dec. 5, 2001 entitled “Method of Collecting Placental Stem Cells” and U.S. application Ser. No. 10/076,180, filed Feb. 13, 2002, entitled “Post-Partum Mammalian Placenta, Its Use and Placental Stem Cells Therefrom,” both of which are incorporated herein by reference in their entireties.

Embryonic-like stem cells are extracted from a drained placenta by means of a perfusion technique that utilizes either or both of the umbilical artery and umbilical vein. The placenta is preferably drained by exsanguination and collection of residual blood (e.g., residual umbilical cord blood). The drained placenta is then processed in such a manner as to establish an ex vivo, natural bioreactor environment in which resident embryonic-like stem cells within the parenchyma and extravascular space are recruited. The embryonic-like stem cells migrate into the drained, empty microcirculation where, according to the methods of the invention, they are collected, preferably by washing into a collecting vessel by perfusion.

4.3. Illustrative JNK and/or MKK Inhibitors

In one embodiment, the JNK inhibitor or MKK inhibitor has the following structure (I):

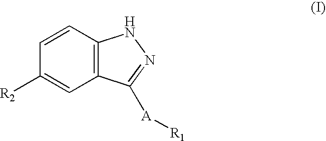

wherein:

A is a direct bond, —(CH₂)ₐ—, —(CH₂)_{b}CH═CH(CH₂)_{c}—, or —(CH₂)_{b}C≡C(CH₂)_{c}—;

R₁ is aryl, heteroaryl or heterocycle fused to phenyl, each being optionally substituted with one to four substituents independently selected from R₃;

R₂ is —R₃, —R₄, —(CH₂)_{b}C(═O)R₅, —(CH₂)_{b}C(═O)OR₅, —(CH₂)_{b}C(═O)NR₅R₆, —(CH₂)_{b}C(═O)NR₅(CH₂)ₙC(═O)R₆, —(CH₂)_{b}NR₅C(═O)R₆, —(CH₂)_{b}NR₅C(═O)NR₆R₇, —(CH₂)_{b}NR₅R₆, —(CH₂)_{b}OR₅, —(CH₂)_{b}SO_{d}R₅ or —(CH₂)_{b}SO₂NR₅R₆;

a is 1, 2, 3, 4, 5 or 6;

b and c are the same or different and at each occurrence independently selected from 0, 1, 2, 3 or 4;

d is at each occurrence 0, 1 or 2;

R₃ is at each occurrence independently halogen, hydroxy, carboxy, alkyl, alkoxy, haloalkyl, acyloxy, thioalkyl, sulfinylalkyl, sulfonylalkyl, hydroxyalkyl, aryl, arylalkyl, heterocycle, heterocycloalkyl, —C(═O)OR₈, —OC(═O)R₈, —C(═O)NR₈R₉, —C(═O)NR₈OR₉, —SO₂NR₈R₉, —NR₈SO₂R₉, —CN, —NO₂, —NR₈R₉, —NR₈C(═O)R₉, —NR₈C(═O)(CH₂)_{b}OR₉, —NR₈C(═O)(CH₂)_{b}R₉, —NR₈C(═O)(CH₂)_{b}NR₈R₉, —O(CH₂)_{b}NR₈R₉, or heterocycle fused to phenyl;

R₄ is alkyl, aryl, arylalkyl, heterocycle or heterocycloalkyl, each being optionally substituted with one to four substituents independently selected from R₃, or R₄ is halogen or hydroxy;

R₅, R₆ and R₇ are the same or different and at each occurrence independently hydrogen, alkyl, aryl, arylalkyl, heterocycle or heterocycloalkyl, wherein each of R₅, R₆ and R₇ are optionally substituted with one to four substituents independently selected from R₃; and

R₈ and R₉ are the same or different and at each occurrence independently hydrogen, alkyl, aryl, arylalkyl, heterocycle, or heterocycloalkyl, or R₈ and R₉ taken together with the atom or atoms to which they are bonded form a heterocycle, wherein each of R₈, R₉, and R₈ and R₉ taken together to form a heterocycle are optionally substituted with one to four substituents independently selected from R₃.

In one embodiment, -A-R₁ is phenyl, optionally substituted with one to four substituents independently selected from halogen, alkoxy, —NR₈C(═O)R₉, —C(═O)NR₈R₉, and —O(CH₂)_{b}NR₈R₉, wherein b is 2 or 3 and wherein R₈ and R₉ are defined above.

In another embodiment, R₂ is —R₄, —(CH₂)_{b}C(═O)R₅, —(CH₂)_{b}C(═O)OR₅, —(CH₂)_{b}C(═O)NR₅R₆, —(CH₂)_{b}C(═O)NR₅(CH₂)ₙC(═O)R₆, —(CH₂)_{b}NR₅C(═O)R₆, —(CH₂)_{b}NR₅C(═O)NR₆R₇, —(CH₂)_{b}NR₅R₆, —(CH₂)_{b}OR₅, —(CH₂)_{b}SO_{d}R₅ or —(CH₂)_{b}SO₂NR₅R₆, and b is an integer ranging from 0-4.

In another embodiment, R₂ is —(CH₂)_{b}C(═O)NR₅R₆, —(CH₂)_{b}NR₅C(═O)R₆, 3-triazolyl or 5-tetrazolyl, wherein b is 0 and wherein R₈ and R₉ are defined above.

In another embodiment, R₂ is 3-triazolyl or 5-tetrazolyl.

In another embodiment:

(a) -A-R₁ is phenyl, optionally substituted with one to four substituents independently selected from halogen, alkoxy, —NR₈C(═O)R₉, —C(═O)NR₈R₉, and —O(CH₂)_{b}NR₈R₉, wherein b is 2 or 3; and

(b) R₂ is —(CH₂)_{b}C(═O)NR₅R₆, —(CH₂)_{b}NR₅C(═O)R₆, 3-triazolyl or 5-tetrazolyl, wherein b is 0 and wherein R₈ and R₉ are defined above.

In another embodiment:

(a) -A-R₁ is phenyl, optionally substituted with one to four substituents independently selected from halogen, alkoxy, —NR₈C(═O)R₉, —C(═O)NR₈R₉, and —O(CH₂)_{b}NR₈R₉, wherein b is 2 or 3; and

(b) R₂ is 3-triazolyl or 5-tetrazolyl.

In another embodiment, R₂ is R₄, and R₄ is 3-triazolyl, optionally substituted at its 5-position with:

(a) a C₁-C₄ straight or branched chain alkyl group optionally substituted with a hydroxyl, methylamino, dimethylamino or 1-pyrrolidinyl group; or

(b) a 2-pyrrolidinyl group.

In another embodiment, R₂ is R₄, and R₄ is 3-triazolyl, optionally substituted at its 5-position with: methyl, n-propyl, isopropyl, 1-hydroxyethyl, 3-hydroxypropyl, methylaminomethyl, dimethylaminomethyl, 1-(dimethylamino)ethyl, 1-pyrrolidinylmethyl or 2-pyrrolidinyl.

In another embodiment, the compounds of structure (I) have structure (IA) when A is a direct bond, or have structure (IB) when A is —(CH₂)ₐ—:

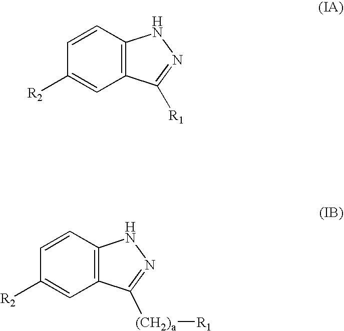

In other embodiments, the compounds of structure (I) have structure (IC) when A is a —CH₂)_{b}CH═CH(CH₂)C—, and have structure (ID) when A is —(CH₂)_{b}C≡C(CH₂)ₙ—:

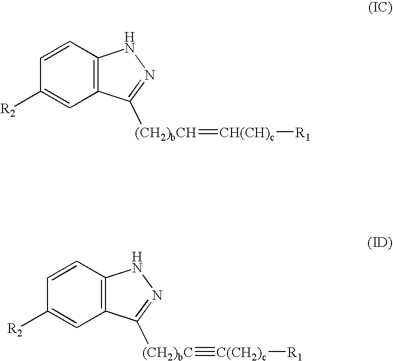

In further embodiments of this invention, R₁ of structure (I) is aryl or substituted aryl, such as phenyl or substituted phenyl as represented by the following structure (IE):

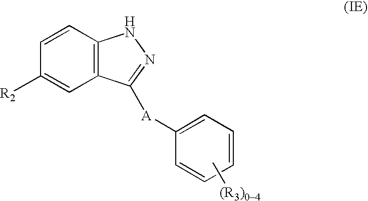

In another embodiment, R₂ of structure (I) is —(CH₂)_{b}NR₅(C═O)R₆. In one aspect of this embodiment, b=0 and the compounds have the following structure (IF):

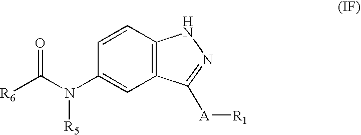

Representative R₂ groups of the compounds of structure (I) include alkyl (such as methyl and ethyl), halo (such as chloro and fluoro), haloalkyl (such as trifluoromethyl), hydroxy, alkoxy (such as methoxy and ethoxy), amino, arylalkyloxy (such as benzyloxy), mono- or di-alkylamine (such as —NHCH₃, —N(CH₃)₂ and —NHCH₂CH₃), —NHC(═O)R₆ wherein R₆ is a substituted or unsubstituted phenyl or heteroaryl (such as phenyl or heteroaryl substituted with hydroxy, carboxy, amino, ester, alkoxy, alkyl, aryl, haloalkyl, halo, —CONH₂ and —CONH alkyl), —NH(heteroarylalkyl) (such as —NHCH₂(3-pyridyl), —NHCH₂(4-pyridyl), heteroaryl (such as pyrazolo, triazolo and tetrazolo), —C(═O)NHR₆ wherein R₆ is hydrogen, alkyl, or as defined above (such as —C(═O)NH₂, —C(═O)NHCH₃, —C(═O)NH(H-carboxyphenyl), —C(═O)N(CH₃)₂), arylalkenyl (such as phenylvinyl, 3-nitrophenylvinyl, 4-carboxyphenylvinyl), heteroarylalkenyl (such as 2-pyridylvinyl, 4-pyridylvinyl).

Representative R₃ groups of the compounds of structure (I) include halogen (such as chloro and fluoro), alkyl (such as methyl, ethyl and isopropyl), haloalkyl (such as trifluoromethyl), hydroxy, alkoxy (such as methoxy, ethoxy, n-propyloxy and isobutyloxy), amino, mono- or di-alkylamino (such as dimethylamine), aryl (such as phenyl), carboxy, nitro, cyano, sulfinylalkyl (such as methylsulfinyl), sulfonylalkyl (such as methylsulfonyl), sulfonamidoalkyl (such as —NHSO₂CH₃), —NR₈C(═O)(CH₂)_{b}OR₉ (such as NHC(═O)CH₂OCH₃), NHC(═O)R₉ (such as —NHC(═O)CH₃, —NHC(═O)CH₂C₆H₅, —NHC(═O)(2-furanyl)), and —O(CH₂)_{b}NR₈R₉ (such as —O(CH₂)₂N(CH₃)₂).

The compounds of structure (I) can be made using organic synthesis techniques known to those skilled in the art, as well as by the methods described in International Publication No. WO 02/10137 (particularly in Examples 1-430, at page 35, line 1 to page 396, line 12), published Feb. 7, 2002, which is incorporated herein by reference in its entirety. Further, specific examples of these compounds are found in this publication.

Illustrative examples of JNK inhibitors or MKK inhibitors of structure (I) are:

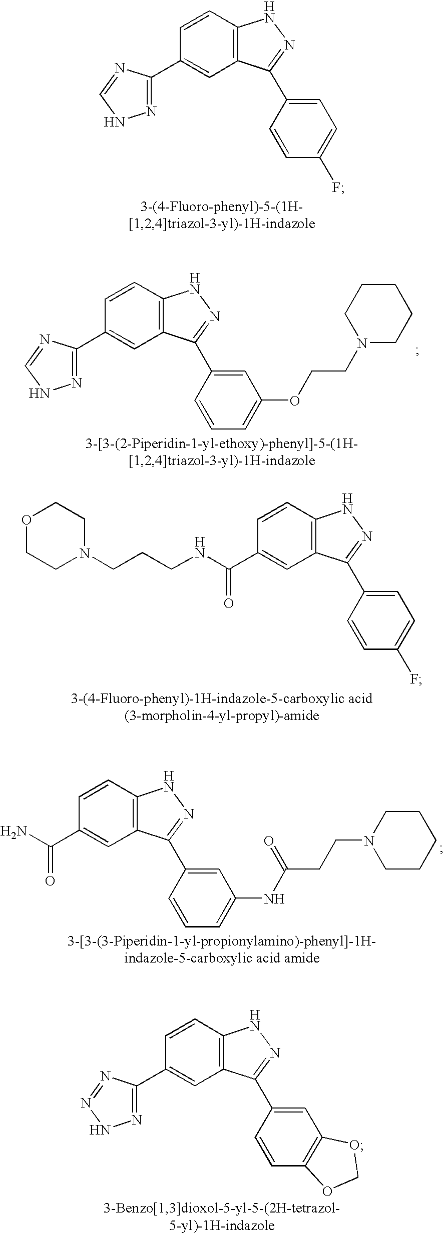

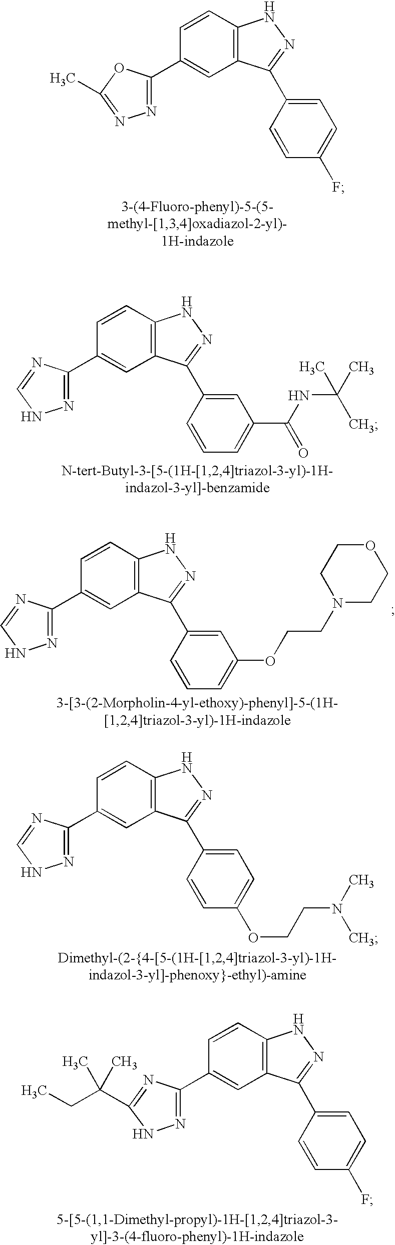

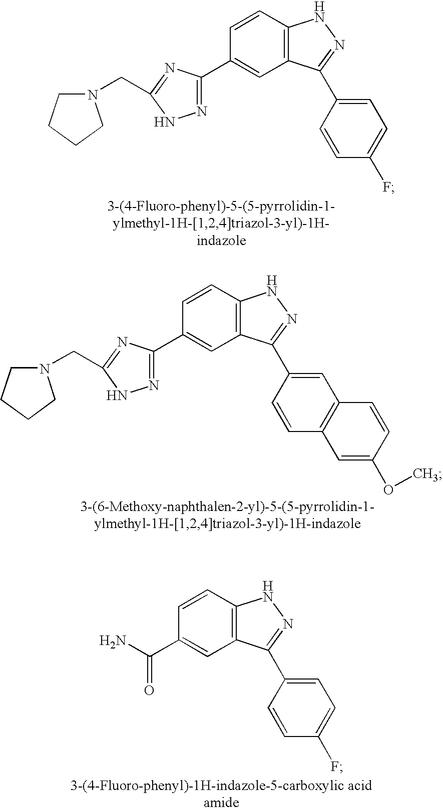

and pharmaceutically acceptable salts thereof.

In another embodiment, the JNK inhibitor or MKK inhibitor has the following structure (II):

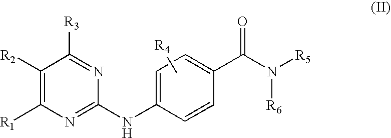

wherein:

R₁ is aryl or heteroaryl optionally substituted with one to four substituents independently selected from R₇;

R₂ is hydrogen;

R₃ is hydrogen or lower alkyl;

R₄ represents one to four optional substituents, wherein each substituent is the same or different and independently selected from halogen, hydroxy, lower alkyl and lower alkoxy;

R₅ and R₆ are the same or different and independently —R₈, —(CH₂)ₐC(═O)R₉, —(CH₂)ₐC(═O)OR₉, —(CH₂)ₐC(═O)NR₉R₁₀, —(CH₂)ₐC(═O)NR₉(CH₂)_{b}C(═O)R₁₀, —(CH₂)ₐNR₉C(═O)R₁₀, (CH₂)ₐNR₁₁C(═O)NR₉R₁₀, —(CH₂)ₐNR₉R₁₀, —(CH₂)ₐOR₉, —(CH₂)ₐSO_{c}R₉ or —(CH₂)ₐSO₂NR₉R₁₀;

or R₅ and R6 taken together with the nitrogen atom to which they are attached to form a heterocycle or substituted heterocycle;

R₇ is at each occurrence independently halogen, hydroxy, cyano, nitro, carboxy, alkyl, alkoxy, haloalkyl, acyloxy, thioalkyl, sulfinylalkyl, sulfonylalkyl, hydroxyalkyl, aryl, arylalkyl, heterocycle, substituted heterocycle, heterocycloalkyl, —C(═O)OR₈, —OC(═O)R₈, —C(═O)NR₈R₉, —C(═O)NR₈OR₉, —SO_{c}R₈, —SO_{c}NR₈R₉, —NR₈SO_{c}R₉, —NR₈R₉, —NR₈C(═O)R₉, —NR₈C(═O)(CH₂)_{b}OR₉, —NR₈C(═O)(CH₂)_{b}R₉, —O(CH₂)_{b}NR₈R₉, or heterocycle fused to phenyl;

R₈, R₉, R₁₀ and R₁₁ are the same or different and at each occurrence independently hydrogen, alkyl, aryl, arylalkyl, heterocycle, heterocycloalkyl;

or R₈ and R₉ taken together with the atom or atoms to which they are attached to form a heterocycle;

a and b are the same or different and at each occurrence independently selected from 0, 1, 2, 3 or 4; and

c is at each occurrence 0, 1 or 2.

In one embodiment, R₁ is a substituted or unsubstituted aryl or heteroaryl. When R₁ is substituted, it is substituted with one or more substituents defined below. In one embodiment, when substituted, R₁ is substituted with a halogen, —SO₂R₈ or —SO₂R₈R₉.

In another embodiment, R₁ is substituted or unsubstituted aryl, furyl, benzofuranyl, thiophenyl, benzothiophenyl, quinolinyl, pyrrolyl, indolyl, oxazolyl, benzoxazolyl, imidazolyl, benzimidazolyl, thiazolyl, benzothiazolyl, isoxazolyl, pyrazolyl, isothiazolyl, pyridazinyl, pyrimidinyl, pyrazinyl, triazinyl, cinnolinyl, phthalazinyl or quinazolinyl.

In another embodiment R₁ is substituted or unsubstituted aryl or heteroaryl. When R₁ is substituted, it is substituted with one or more substituents defined below. In one embodiment, when substituted, R₁ is substituted with a halogen, —SO₂R₈ or —SO₂R₈R₉.

In another embodiment, R₁ is substituted or unsubstituted aryl, preferably phenyl. When R₁ is a substituted aryl, the substituents are defined below. In one embodiment, when substituted, R₁ is substituted with a halogen, —SO₂R₈ or —SO₂R₈R₉.

In another embodiment, R₅ and R₆, taken together with the nitrogen atom to which they are attached form a substituted or unsubstituted nitrogen-containing non-aromatic heterocycle, in one embodiment, piperazinyl, piperidinyl or morpholinyl.

When R₅ and R₆, taken together with the nitrogen atom to which they are attached form substituted piperazinyl, piperadinyl or morpholinyl, the piperazinyl, piperadinyl or morpholinyl is substituted with one or more substituents defined below. In one embodiment, when substituted, the substituent is alkyl, amino, alkylamino, alkoxyalkyl, acyl, pyrrolidinyl or piperidinyl.

In one embodiment, R₃ is hydrogen and R₄ is not present, and the JNK inhibitor or MKK inhibitor has the following structure (IIA):

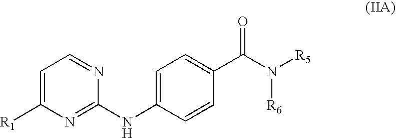

and pharmaceutically acceptable salts thereof.

In a more specific embodiment, R₁ is phenyl optionally substituted with R₇, and having the following structure (IIB):

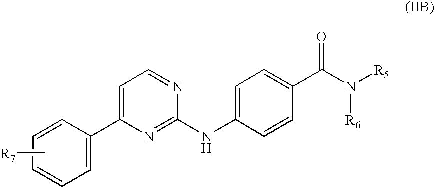

and pharmaceutically acceptable salts thereof.

In still a further embodiment, R₇ is at the para position of the phenyl group relative to the pyrimidine, as represented by the following structure (IIC):

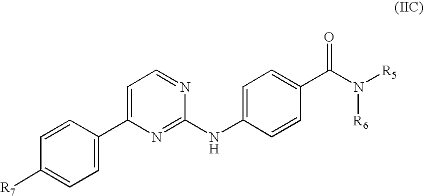

and pharmaceutically acceptable salts thereof.

The JNK inhibitors or MKK inhibitors of structure (II) can be made using organic synthesis techniques known to those skilled in the art, as well as by the methods described in International Publication No. WO 02/46170 (particularly Examples 1-27 at page 23, line 5 to page 183, line 25), published Jun. 13, 2002, which is hereby incorporated by reference in its entirety. Further, specific examples of these compounds are found in the publication.

Illustrative examples of JNK inhibitors or MKK inhibitors of structure (II) are:

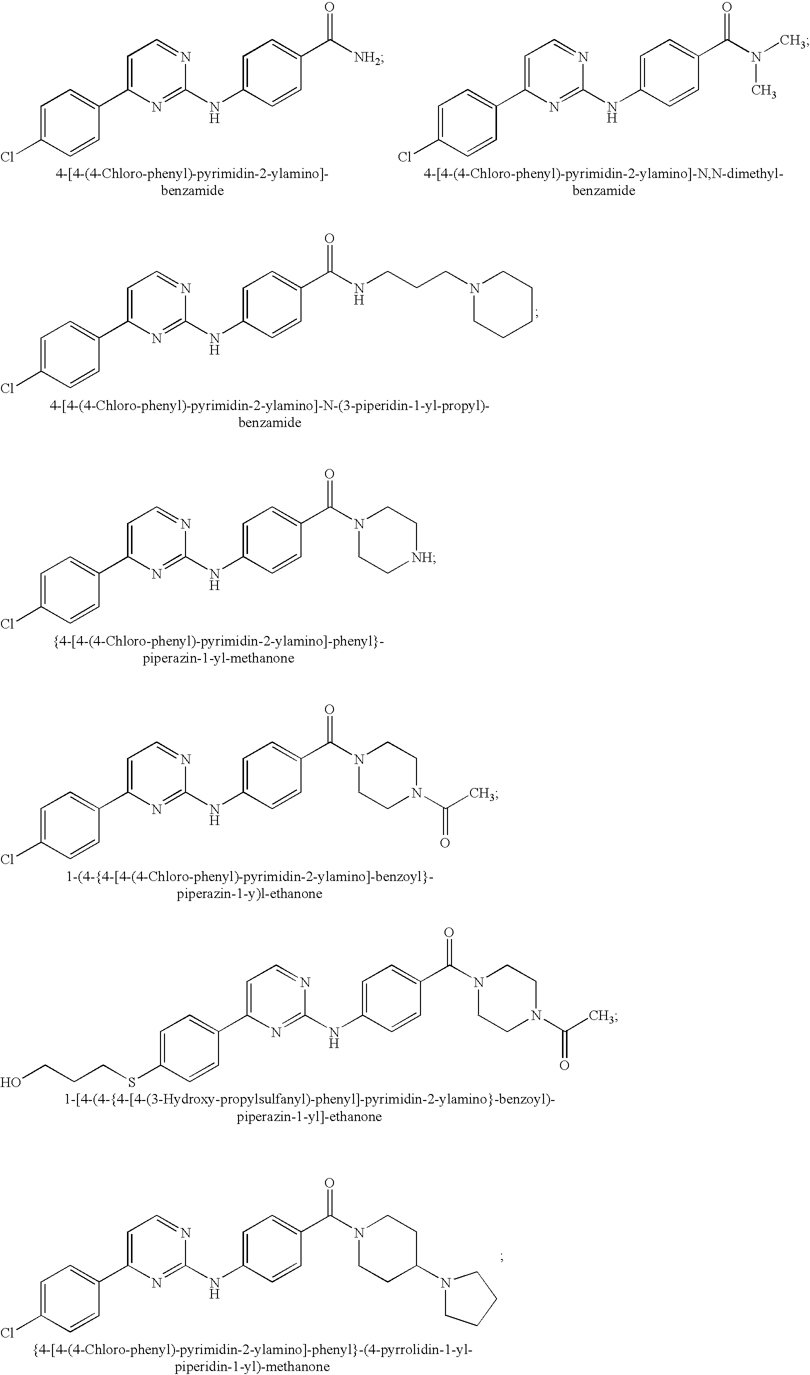

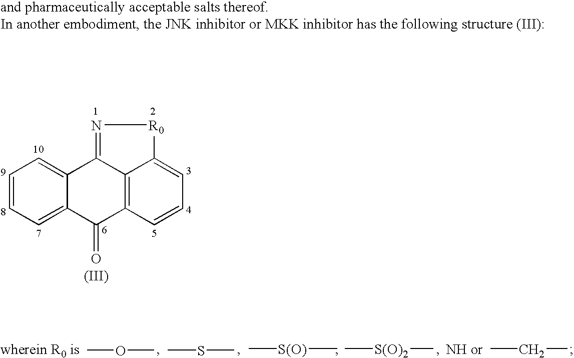

the compound of structure (III) being: (i) unsubstituted, (ii) monosubstituted and having a first substituent, or (iii) disubstituted and having a first substituent and a second substituent;

the first or second substituent, when present, is at the 3, 4, 5, 7, 8, 9, or 10 position, wherein the first and second substituent, when present, are independently alkyl, hydroxy, halogen, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono-alkylaminoalkoxy, di-alkylaminoalkoxy, or a group represented by structure (a), (b), (c), (d), (e), or (f):

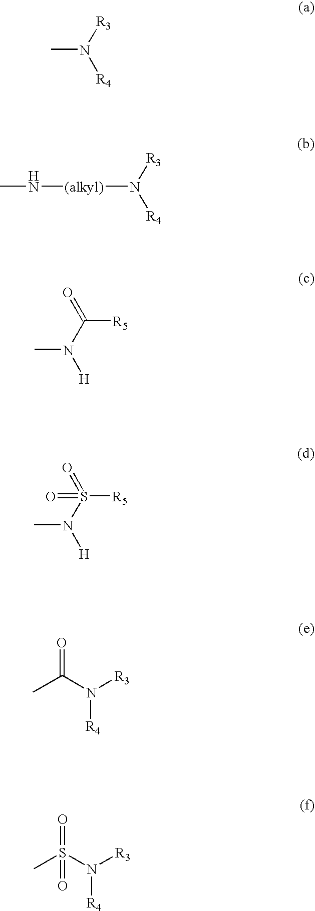

wherein R₃ and R₄ are taken together and represent alkylidene or a heteroatom-containing cyclic alkylidene or R₃ and R₄ are independently hydrogen, alkyl, cycloalkyl, aryl, arylalkyl, cycloalkylalkyl, aryloxyalkyl, alkoxyalkyl, aminoalkyl, mono-alkylaminoalkyl, or di-alkylaminoalkyl; and

R₅ is hydrogen, alkyl, cycloalkyl, aryl, arylalkyl, cycloalkylalkyl, alkoxy, alkoxyalkyl, alkoxycarbonylalkyl, amino, mono-alkylamino, di-alkylamino, arylamino, arylalkylamino, cycloalkylamino, cycloalkylalkylamino, aminoalkyl, mono-alkylaminoalkyl, or di-alkylaminoalkyl.

In another embodiment, the JNK inhibitor or MKK inhibitor has the following structure (IIIA):

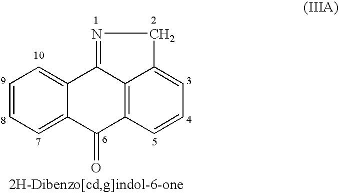

being: (i) unsubstituted, (ii) monosubstituted and having a first substituent, or (iii) disubstituted and having a first substituent and a second substituent;

the first or second substituent, when present, is at the 3, 4, 5, 7, 8, 9, or 10 position;

wherein the first and second substituent, when present, are independently alkyl, hydroxy, halogen, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono-alkylaminoalkoxy, di-alkylaminoalkoxy, or a group represented by structure (a), (b), (c), (d), (e), or (f):

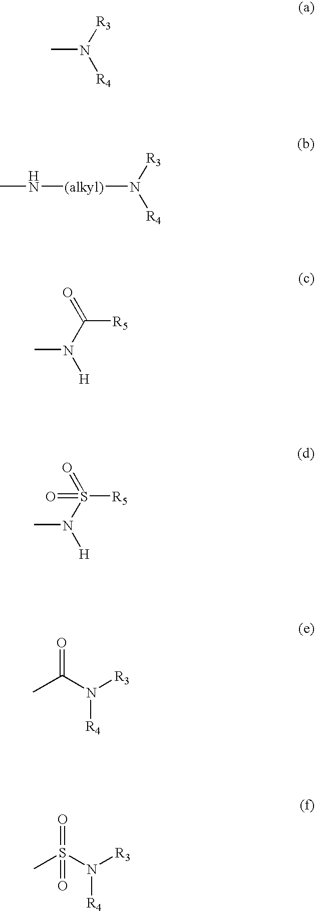

wherein R₃ and R₄ are taken together and represent alkylidene or a heteroatom-containing cyclic alkylidene or R₃ and R₄ are independently hydrogen, alkyl, cycloalkyl, aryl, arylalkyl, cycloalkylalkyl, aryloxyalkyl, alkoxyalkyl, aminoalkyl, mono-alkylaminoalkyl, or di-alkylaminoalkyl; and

R₅ is hydrogen, alkyl, cycloalkyl, aryl, arylalkyl, cycloalkylalkyl, alkoxy, alkoxyalkyl, alkoxycarbonylalkyl, amino, mono-alkylamino, di-alkylamino, arylamino, arylalkylamino, cycloalkylamino, cycloalkylalkylamino, aminoalkyl, mono-alkylaminoalkyl, or di-alkylaminoalkyl.

A subclass of the compounds of structure (IIIA) is that wherein the first or second substituent is present at the 5, 7, or 9 position. In one embodiment, the first or second substituent is present at the 5 or 7 position.

A second subclass of compounds of structure (IIIA) is that wherein the first or second substituent is present at the 5, 7, or 9 position;

the first or second substituent is independently alkoxy, aryloxy, aminoalkyl, mono-alkylaminoalkyl, di-alkylaminoalkyl, or a group represented by the structure (a), (c), (d), (e), or (f);

R₃ and R₄ are independently hydrogen, alkyl, cycloalkyl, aryl, arylalkyl, or cycloalkylalkyl; and

R₅ is hydrogen, alkyl, cycloalkyl, aryl, arylalkyl, or cycloalkylalkyl.

In another embodiment, the JNK inhibitor or MKK inhibitor has the following structure (IIIB):

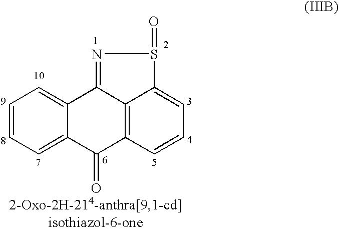

being (i) unsubstituted, (ii) monosubstituted and having a first substituent, or (ii) disubstituted and having a first substituent and a second substituent;

the first or second substituent, when present, is at the 3, 4, 5, 7, 8, 9, or 10 position;

wherein the first and second substituent, when present, are independently alkyl, halogen, hydroxy, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono-alkylaminoalkoxy, di-alkylaminoalkoxy, or a group represented by structure (a), (b) (c), (d), (e), or (f):

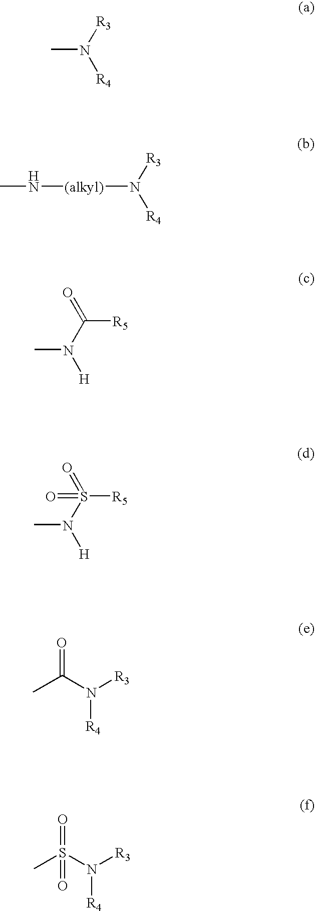

wherein R₃ and R₄ are taken together and represent alkylidene or a heteroatom-containing cyclic alkylidene or R₃ and R₄ are independently hydrogen, alkyl, cycloalkyl, aryl, arylalkyl, cycloalkylalkyl, aryloxyalkyl, alkoxyalkyl, aminoalkyl, mono-alkylaminoalkyl, or di-alkylaminoalkyl; and

R₅ is hydrogen, alkyl, cycloalkyl, aryl, arylalkyl, cycloalkylalkyl, alkoxy, alkoxyalkyl, alkoxycarbonylalkyl, amino, mono-alkylamino, di-alkylamino, arylamino, arylalkylamino, cycloalkylamino, cycloalkylalkylamino, aminoalkyl, mono-alkylaminoalkyl, or di-alkylaminoalkyl.

A subclass of the compounds of structure (IIIB) is that wherein the first or second substituent is present at the 5, 7, or 9 position. In one embodiment, the first or second substituent is present at the 5 or 7 position.

A second subclass of the compounds of structure (IIIB) is that wherein the first or second substituent is independently alkoxy, aryloxy, or a group represented by the structure (a), (c), (d), (e), or (f);

R₃ and R₄ are independently hydrogen, alkyl, cycloalkyl, aryl, arylalkyl, or cycloalkylalkyl; and

R₅ is hydrogen, alkyl, cycloalkyl, aryl, arylalkyl, or cycloalkylalkyl.

In another embodiment, the JNK inhibitor or MKK inhibitor has the following structure (IIIC):

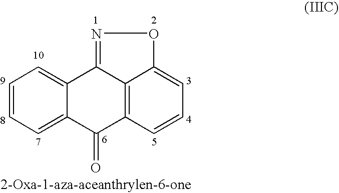

being (i) monosubstituted and having a first substituent or (ii) disubstituted and having a first substituent and a second substituent;

the first or second substituent, when present, is at the 3, 4, 5, 7, 8, 9, or 10 position;

wherein the first and second substituent, when present, are independently alkyl, halogen, hydroxy, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono-alkylaminoalkoxy, di-alkylaminoalkoxy, or a group represented by structure (a), (b), (c) (d), (e), or (f):

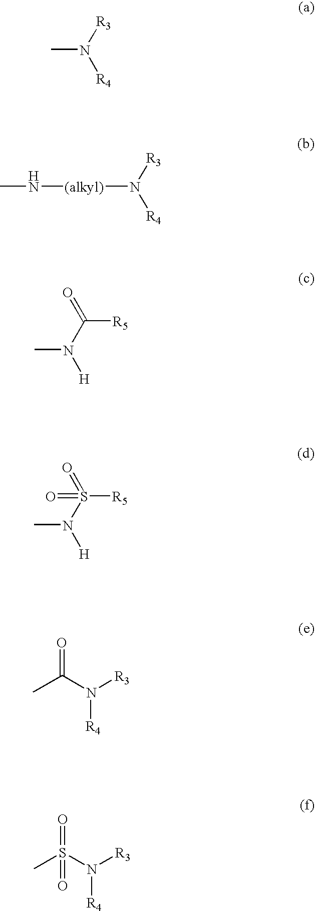

wherein R₃ and R₄ are taken together and represent alkylidene or a heteroatom-containing cyclic alkylidene or R₃ and R₄ are independently hydrogen, alkyl, cycloalkyl, aryl, arylalkyl, cycloalkylalkyl, aryloxyalkyl, alkoxyalkyl, aminoalkyl, mono-alkylaminoalkyl, or di-alkylaminoalkyl; and

R₅ is hydrogen, alkyl, cycloalkyl, aryl, arylalkyl, cycloalkylalkyl, alkoxy, alkoxyalkyl, alkoxycarbonylalkyl, amino, mono-alkylamino, di-alkylamino, arylamino, arylalkylamino, cycloalkylamino, cycloalkylalkylamino, aminoalkyl, mono-alkylaminoalkyl, or di-alkylaminoalkyl.

A subclass of the compounds of structure (IIIC) is that wherein the first or second substituent is present at the 5, 7, or 9 position. In one embodiment, the first or second substituent is present at the 5 or 7 position.

A second subclass of the compounds of structure (IIIC) is that wherein the first or second substituent is independently alkoxy, aryloxy, aminoalkyl, mono-alkylaminoalkyl, di-alkylaminoalkyl, or a group represented by the structure (a), (c), (d), (e), or (f);

R₃ and R₄ are independently hydrogen, alkyl, cycloalkyl, aryl, arylalkyl, or cycloalkylalkyl; and

R₅ is hydrogen, alkyl, cycloalkyl, aryl, arylalkyl, or cycloalkylalkyl.

In another embodiment, the JNK inhibitor or MKK inhibitor has the following structure (IIID):

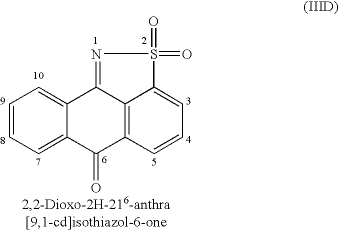

being (i) monosubstituted and having a first substituent present at the 5, 7, or 9 position, (ii) disubstituted and having a first substituent present at the 5 position and a second substituent present at the 7 position, (iii) disubstituted and having a first substituent present at the 5 position and a second substituent present at the 9 position, or (iv) disubstituted and having a first substituent present at the 7 position and a second substituent present at the 9 position;

wherein the first and second substituent, when present, are independently alkyl, halogen, hydroxy, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono-alkylaminoalkoxy, di-alkylaminoalkoxy, or a group represented by structure (a), (b), (c), (d), (e), or (f):

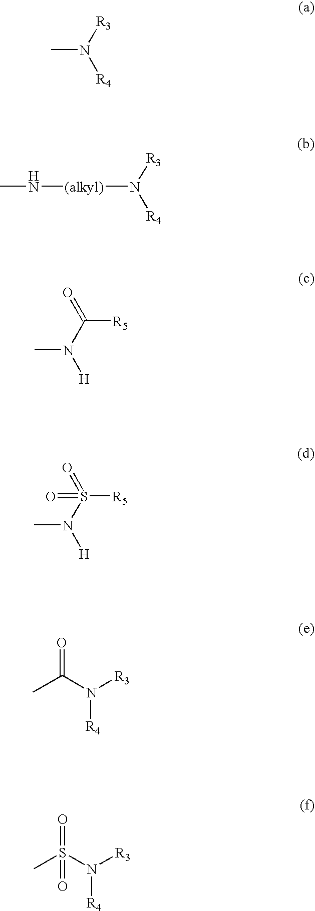

wherein R₃ and R₄ are taken together and represent alkylidene or a heteroatom-containing cyclic alkylidene or R₃ and R₄ are independently hydrogen, alkyl, cycloalkyl, aryl, arylalkyl, cycloalkylalkyl, aryloxyalkyl, alkoxyalkyl, aminoalkyl, mono-alkylaminoalkyl, or di-alkylaminoalkyl; and

R₅ is hydrogen, alkyl, cycloalkyl, aryl, arylalkyl, cycloalkylalkyl, alkoxy, alkoxyalkyl, alkoxycarbonylalkyl, amino, mono-alkylamino, di-alkylamino, arylamino, arylalkylamino, cycloalkylamino, cycloalkylalkylamino, aminoalkyl, mono-alkylaminoalkyl, or di-alkylaminoalkyl.

A subclass of the compounds of structure (IIID) is that wherein the first or second substituent is present at the 5 or 7 position.

A second subclass of the compounds of structure (IIID) is that wherein the first or second substituent is independently alkyl, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono-alkylaminoalkoxy, di-alkylaminoalkoxy, or a group represented by structure (a), (c), (d), (e), or (f).

Another subclass of the compounds of structure (IIID) is that wherein the first and second substituent are independently alkoxy, aryloxy, or a group represented by the structure (a), (c), (d), (e), or (f);

R₃ and R₄ are independently hydrogen, alkyl, cycloalkyl, aryl, arylalkyl, or cycloalkylalkyl; and

R₅ is hydrogen, alkyl, cycloalkyl, aryl, arylalkyl, alkoxycarbonyl, or cycloalkylalkyl.

In another embodiment, the JNK inhibitor or MKK inhibitor has the following structure (IIIE):

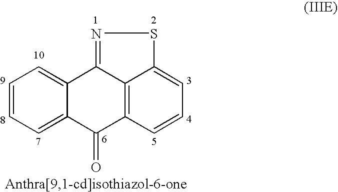

being (i) monosubstituted and having a first substituent present at the 5, 7, or 9 position, (ii) disubstituted and having a first substituent present at the 5 position and a second substituent present at the 9 position, (iii) disubstituted and having a first substituent present at the 7 position and a second substituent present at the 9 position, or (iv) disubstituted and having a first substituent present at the 5 position and a second substituent present at the 7 position;

wherein the first and second substituent, when present, are independently alkyl, halogen, hydroxy, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono-alkylaminoalkoxy, di-alkylaminoalkoxy, or a group represented by structure (a), (b), (c), (d), (e), or (f):

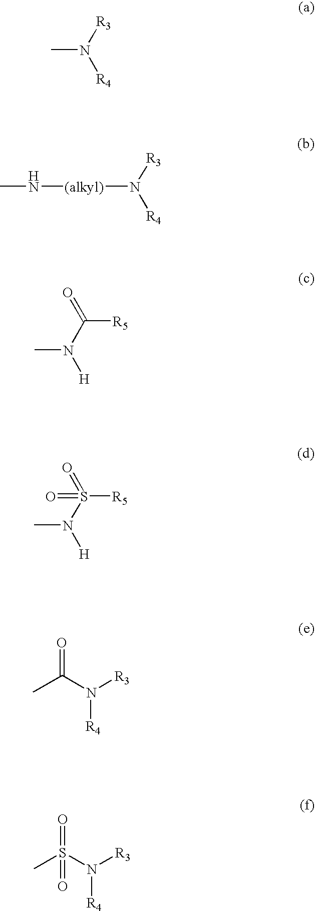

wherein R₃ and R₄ are taken together and represent alkylidene or a heteroatom-containing cyclic alkylidene or R₃ and R₄ are independently hydrogen, alkyl, cycloalkyl, aryl, arylalkyl, cycloalkylalkyl, aryloxyalkyl, alkoxyalkyl, aminoalkyl, mono-alkylaminoalkyl, or di-alkylaminoalkyl; and

R₅ is hydrogen, alkyl, cycloalkyl, aryl, arylalkyl, cycloalkylalkyl, alkoxy, alkoxyalkyl, alkoxycarbonylalkyl, amino, mono-alkylamino, di-alkylamino, arylamino, arylalkylamino, cycloalkylamino, cycloalkylalkylamino, aminoalkyl, mono-alkylaminoalkyl, or di-alkylaminoalkyl.

A subclass of the compounds of structure (IIIE) is that wherein the first or second substituent is present at the 5 or 7 position.

A second subclass of the compounds of structure (IIIE) is that wherein the compound of structure (IIIE) is disubstituted and at least one of the substituents is a group represented by the structure (d) or (f).

Another subclass of the compounds of structure (IIIE) is that wherein the compounds are monosubstituted. Yet another subclass of compounds is that wherein the compounds are monosubstituted at the 5 or 7 position with a group represented by the structure (e) or (f).

In another embodiment, the JNK inhibitor or MKK inhibitor has the following structure (IIIF):

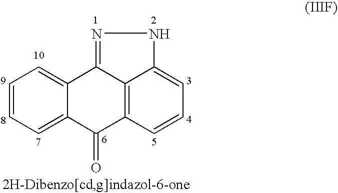

being (i) unsubstituted, (ii) monosubstituted and having a first substituent, or (iii) disubstituted and having a first substituent and a second substituent;

the first or second substituent, when present, is at the 3, 4, 5, 7, 8, 9, or 10 position;

wherein the first and second substituent, when present, are independently alkyl, hydroxy, halogen, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono-alkylaminoalkoxy, di-alkylaminoalkoxy, or a group represented by structure (a), (b), (c), (d), (e), or (f):

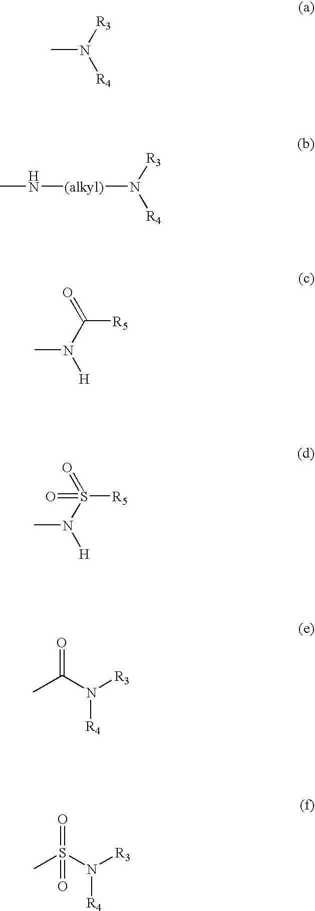

wherein R₃ and R₄ are taken together and represent alkylidene or a heteroatom-containing cyclic alkylidene or R₃ and R₄ are independently hydrogen, alkyl, cycloalkyl, aryl, arylalkyl, cycloalkylalkyl, aryloxyalkyl, alkoxyalkyl, aminoalkyl, mono-alkylaminoalkyl, or di-alkylaminoalkyl; and

R₅ is hydrogen, alkyl, cycloalkyl, aryl, arylalkyl, cycloalkylalkyl, alkoxy, alkoxyalkyl, alkoxycarbonylalkyl, amino, mono-alkylamino, di-alkylamino, arylamino, arylalkylamino, cycloalkylamino, cycloalkylalkylamino, aminoalkyl, mono-alkylaminoalkyl, or di-alkylaminoalkyl.

In one embodiment, the compound of structure (IIIF), or a pharmaceutically acceptable salt thereof is unsubstituted at the 3, 4, 5, 7, 8, 9, or 10 position.

The JNK inhibitors or MKK inhibitors of structure (III) can be made using organic synthesis techniques known to those skilled in the art, as well as by the methods described in International Publication No. WO 01/12609 (particularly Examples 1-7 at page 24, line 6 to page 49, line 16), published Feb. 22, 2001, as well as International Publication No. WO 02/066450 (particularly compounds AA-HG at pages 59-108), published Aug. 29, 2002, each of which is hereby incorporated by reference in its entirety. Further, specific examples of these compounds can be found in the publications.

Illustrative examples of JNK inhibitors or MKK inhibitors of structure (III) are:

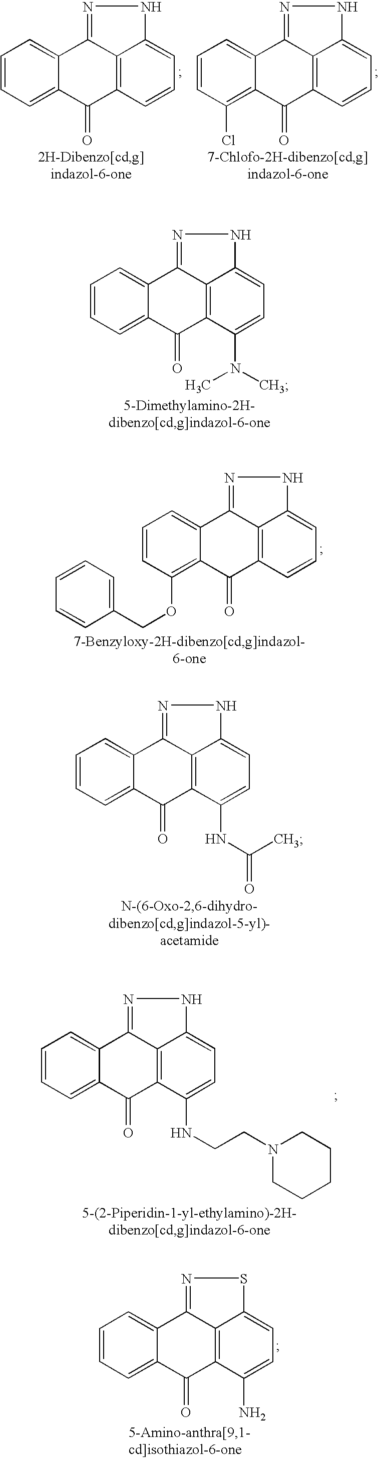

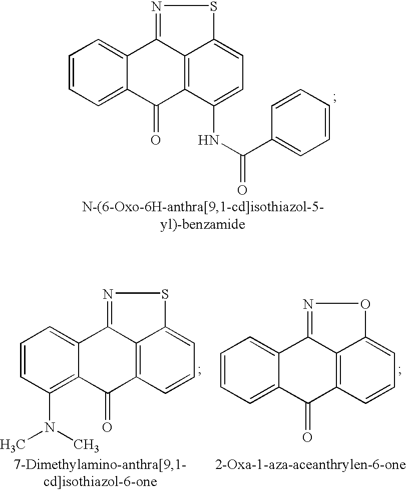

and pharmaceutically acceptable salts thereof.

Other JNK inhibitors or MKK inhibitors that are useful in the present methods include, but are not limited to, those disclosed in International Publication No. WO 00/39101, (particularly at page 2, line 10 to page 6, line 12); International Publication No. WO 01/14375 (particularly at page 2, line 4 to page 4, line 4); International Publication No. WO 00/56738 (particularly at page 3, line 25 to page 6, line 13); International Publication No. WO 01/27089 (particularly at page 3, line 7 to page 5, line 29); International Publication No. WO 00/12468 (particularly at page 2, line 10 to page 4, line 14); European Patent Publication 1 110 957 (particularly at page 19, line 52 to page 21, line 9); International Publication No. WO 00/75118 (particularly at page 8, line 10 to page 11, line 26); International Publication No. WO 01/12621 (particularly at page 8, line 10 to page 10, line 7); International Publication No. WO 00/64872 (particularly at page 9, line 1 to page, 106, line 2); International Publication No. WO 01/23378 (particularly at page 90, line 1 to page 91, line 11); International Publication No. WO 02/16359 (particularly at page 163, line 1 to page 164, line 25); U.S. Pat. No. 6,288,089 (particularly at column 22, line 25 to column 25, line 35); U.S. Pat. No. 6,307,056 (particularly at column 63, line 29 to column 66, line 12); International Publication No. WO 00/35921 (particularly at page 23, line 5 to page 26, line 14); International Publication No. WO 01/91749 (particularly at page 29, lines 1-22); International Publication No. WO 01/56993 (particularly in at page 43 to page 45); and International Publication No. WO 01/58448 (particularly in at page 39), each of which is incorporated by reference herein in its entirety.

Pharmaceutical compositions including dosage forms of the invention, which comprise an effective amount of a JNK inhibitor or MKK inhibitor can be used in the methods of the invention.

4.4. Methods of Stem Cell Culture

In certain embodiments of the invention, stem or progenitor cells, including but not limited to embryonic stem cells, embryonic-like stem cells, progenitor cells, pluripotent cells, totipotent cells, multipotent cells, cells endogenous to a postpartum perfused placenta, cord blood cells, stem or progenitor cells derived from peripheral blood or adult blood, or bone marrow cells, are exposed to the compounds of the invention and induced to differentiate. These cells may be propagated in vitro using methods well known in the art, or alternatively, may be propagated in a postpartum perfused placenta. See U.S. Application Publication No. US 2003/0032179, published Feb. 13, 2003, entitled “Post-Partum Mammalian Placenta, Its Use and Placental Stem Cells Therefrom” which is hereby incorporated in its entirety.

In certain embodiments, cells endogenous to a postpartum perfused placenta may be collected from the placenta and culture medium and cultured in vitro under conditions appropriate, and for a time sufficient, to induce differentiation to the desired cell type or lineage.

In another embodiment of the invention, the stem or progenitor cells are not derived from a postpartum perfused placenta but instead, are isolated from other sources such as cord blood, bone marrow, peripheral blood or adult blood, are exposed to the compounds of the invention and induced to differentiate. In a preferred embodiment, the differentiation is conducted in vitro under conditions appropriate, and for a time sufficient, to induce differentiation into the desired lineage or cell type. The compounds of the invention are used in the differentiation/culture media by addition, in situ generation, or in any other manner that permits contact of the stem or progenitor cells with the compounds of the invention.

In another embodiment, the cultured stem cells, e.g., stem cells cultured in vitro or in a postpartum perfused placenta, are stimulated to proliferate in culture, for example, by administration of erythropoietin, cytokines, lymphokines, interferons, colony stimulating factors (CSFs), interferons, chemokines, interleukins, recombinant human hematopoietic growth factors including ligands, stem cell factors, thrombopoeitin (Tpo), interleukins, and granulocyte colony-stimulating factor (G-CSF) or other growth factors.

After collection and/or isolation of the cultured cells, they may be identified and characterized by a colony forming unit assay, which is commonly known in the art, such as Mesen Cult™ medium (stem cell Technologies, Inc., Vancouver British Columbia).

In accordance with the present invention, known methods of obtaining stem sells may be applied to generate populations of stem cells which may be differentiated in accordance with the methods of the invention. Caplan et al. (U.S. Pat. No. 5,486,359, entitled “Human mesenchymal stem cells,” issued Jan. 23, 1996, which is incorporated herein by reference in its entirety), discloses methods for obtaining human mesenchymal stem cell (hMSC) compositions derived from bone marrow that serve as the progenitors for mesenchymal cell lineages. Homogeneous hMSC compositions are obtained by positive selection of adherent marrow or periosteal cells that are free of markers associated with either hematopoietic cell or differentiated mesenchymal cells.

Hu et al. (WO 00/73421, entitled “Methods of isolation, cryopreservation, and therapeutic use of human amniotic epithelial cells,” published Dec. 7, 2000 incorporated herein by reference in its entirety) discloses methods for harvesting human amniotic epithelial cells from placenta at delivery, such that the cells are isolated, cultured, cryopreserved for future use, or induced to differentiate. According to Hu et al., a placenta is harvested immediately after delivery and the amniotic membrane separated from the chorion, e.g., by dissection. Amniotic epithelial cells are isolated from the amniotic membrane according to standard cell isolation techniques. The disclosed cells can be cultured in various media, expanded in culture, cryopreserved, or induced to differentiate.

Umbilical cord blood (cord blood) is a known alternative source of hematopoietic progenitor stem cells. Stem cells from cord blood are routinely harvested and cryopreserved for use in hematopoictic reconstitution, a widely used therapeutic procedure used in bone marrow and other related transplantations (see, e.g., Boyse et al., U.S. Pat. No. 5,004,681, entitled “Preservation of Fetal and Neonatal Hematopoietin Stem and Progenitor Cells of the Blood,” Boyse et al., U.S. Pat. No. 5,192,553 entitled, “Isolation and preservation of fetal and neonatal hematopoietic stem and progenitor cells of the blood and methods of therapeutic use,” issued Mar. 9, 1993, each of which is incorporated herein by reference in its entirety). Conventional techniques for the collection of cord blood are based on the use of a needle or cannula, which is used with the aid of gravity to drain cord blood from (i.e., exsanguinate) the placenta (Boyse et al., U.S. Pat. No. 5,192,553, issued Mar. 9, 1993; Boyse et al., U.S. Pat. No. 5,004,681, issued Apr. 2, 1991; Anderson, U.S. Pat. No. 5,372,581, entitled “Method and apparatus for placental blood collection,” issued Dec. 13, 1994; Hessel et al., U.S. Pat. No. 5,415,665, entitled “Umbilical cord clamping, cutting, and blood collecting device and method,” issued May 16, 1995, each of which is incorporated herein by reference in its entirety). The needle or cannula is usually placed in the umbilical vein and the placenta is gently massaged to aid in draining cord blood from the placenta.

Körbling et al. (2002, “Hepatocytes and epithelial cells of donor origin in recipients of peripheral-blood stem cells,” N. Engl. J. Med. 346(10):738-46, which is incorporated herein by reference in its entirety) disclose that stem cells can be procured from peripheral blood and can serve as a source of stem cells that can differentiate into cells of the liver, gastrointestinal tract, and skin.

Naughton et al. (U.S. Pat. No. 5,962,325 entitled “Three-dimensional stromal tissue cultures” issued Oct. 5, 1999) discloses that fetal cells, including fibroblast-like cells and chondrocyte-progenitors, may be obtained from umbilical cord or placenta tissue or umbilical cord blood.

4.4.1. Stem Cell Culture In Vitro

The methods of the invention encompass the regulation of stem cell or progenitor cell differentiation in vitro, comprising incubating the cells with a compound, such as a small organic molecule of the present invention, in vitro, that induces them to differentiate into cells of a particular desired cell lineage, followed by direct transplantation of the differentiated cells to a subject. In a preferred embodiment, the cells are induced to differentiate into a hematopoictic cell lineage.

Methods for culturing stem or progenitor cells in vitro are well known in the art, e.g., see, Thomson et al., 1998, Science 282:1145-47 (embryonic stem cells); Hirashima et al., 1999, Blood 93(4): 1253-63, and Hatzopoulos et al., 1998, Development 125:1457-1468 (endothelial cell progenitors); Slager et al., 1993, Dev. Genet. 14(3):212-24 (neuron or muscle progenitors); Genbachev et al., 1995, Reprod. Toxicol. 9(3):245-55 (cytotrophoblasts, i.e., placental epithelial cell progenitors); Nadkarni et al. 1984, Tumori 70:503-505, Melchner et al., 1985, Blood 66(6): 1469-1472, international application publication WO 00/27999 published May 18, 2000, Himori et al., 1984, Intl. J. Cell Cloning 2:254-262, and Douay et al., 1995, Bone Marrow Transplantation 15:769-775 (hematopoietic progenitor cells); Shamblott et al., 1998, Proc. Natl. Acad. Sci. USA 95:13726-31 (primordial germ cells); Yan et al., 2001, Devel. Biol. 235:422-432 (trophoblast stem cells).

In certain embodiments, the cultured progenitor or stem cells of interest are exposed in vitro to a 0.1 μg/ml, 0.2 μg/ml, 0.3 μg/ml, 0.4 μg/ml, 0.5 μg/ml, 1 μg/ml, 5 μg or 10 μg/ml concentration of a compound of the invention. Preferably the cells of interest are exposed to a concentration of a JNK or MKK inhibitor of between 0.005 μg/ml and 5 mg/ml, more preferably between 1.0 μg/ml and 2 mg/ml.

4.4.2. Stem Cell Culture in a Postpartum Perfused Placenta

4.4.2.1. Pretreatment of Placenta

According to the methods of the invention, a human placenta is recovered shortly after its expulsion after birth and, in certain embodiments, the cord blood in the placenta is recovered. In certain embodiments, the placenta is subjected to a conventional cord blood recovery process. A needle or cannula is typically used, with the aid of gravity, to drain cord blood from (i.e., exsanguinate) the placenta (Boyse et al., U.S. Pat. No. 5,192,553, issued Mar. 9, 1993; Boyse et al., U.S. Pat. No. 5,004,681, issued Apr. 2, 1991; Anderson, U.S. Pat. No. 5,372,581, issued Dec. 13, 1994; Hessel et al., U.S. Pat. No. 5,415,665, entitled Umbilical cord clamping, cutting, and blood collecting device and method, issued May 16, 1995). Such cord blood recovery may be obtained commercially, e.g., LifeBank USA (Cedar Knolls, N.J.), ViaCord (Boston Mass.), Cord Blood Registry (San Bruno, Calif.) and Cryocell (Clearwater, Fla.). The cord blood can be drained shortly after expulsion of the placenta.

Postpartum the placenta is drained of cord blood. The placenta stored may be under sterile conditions and at either room temperature or at a temperature of 5 to 25° C. (centigrade). The placenta may be stored for a period of longer than forty eight hours, and preferably for a period of four to twenty-four hours prior to perfusing the placenta to remove any residual cord blood.

The placenta is preferably recovered after expulsion under aseptic conditions, and stored in an anticoagulant solution at a temperature of 5 to 25° C. (centigrade). Suitable anticoagulant solutions are well known in the art. For example, a solution of heparin or warfarin sodium can be used, e.g., a solution of heparin (1% w/w in 1:1000 solution). The drained placenta is preferably stored for no more than 36 hours before the embryonic-like stem cells are collected. The solution which is used to perfuse the placenta to remove residual cells can be the same solution used to perfuse and culture the placenta for the recovery of stem cells. Any of these perfusates may be collected and used as a source of embryonic-like stem cells.

The placenta may also be recovered from a patient by informed consent and a complete medical history of the patient prior to, during and after pregnancy is also taken and is associated with the placenta. These medical records can be used to coordinate subsequent use of the placenta or the stem cells harvested therefrom. For example, the human placental stem cells can then easily be used for personalized medicine for the infant in question, the parents, siblings or other relatives. Indeed, the human placental stem cells are more versatile than cord blood. However, it should be noted that the invention includes the addition of human placental stem cells produced by the exsanguinated, perfused and/or cultured placenta to cord blood from the same or different placenta and umbilical cord. The resulting cord blood will have an increased concentration/population of human stem cells and thereby is more useful for transplantation e.g. for bone marrow transplantations.

4.4.2.2. Exsanguination of Placenta and Removal of Residual Cells

According to certain embodiments of the invention, stem or progenitor cells, including, but not limited to embryonic-like stem cells, may be recovered from a placenta that is exsanguinated, i.e., completely drained of the cord blood remaining after birth and/or a conventional cord blood recovery procedure. As mentioned above, the placenta may be exsanguinated and perfused as disclosed in co-pending U.S. application Ser. No. 10/004,942, filed Dec. 5, 2001 entitled “Method of Collecting Placental Stem Cells” and U.S. application Ser. No. 10/076,180, filed Feb. 13, 2002, entitled “Post-Partum Mammalian Placenta, Its Use and Placental Stem Cells Therefrom,” both of which are incorporated herein by reference in their entireties.

4.4.2.3. Culture of Placenta and Stem Cells Therein

After exsanguination and a sufficient time of perfusion of the placenta, the embryonic-like stem cells are observed to migrate into the exsanguinated and perfused microcirculation of the placenta where, according to the methods of the invention, they are collected, preferably by washing into a collecting vessel by perfusion. Perfusing the isolated placenta not only serves to remove residual cord blood but also provide the placenta with the appropriate nutrients, including oxygen. The placenta may be cultivated and perfused with a similar solution which was used to remove the residual cord blood cells, preferably, without the addition of anticoagulant agents.

In certain embodiments of the invention, the drained, exsanguinated placenta is cultured as a bioreactor, i.e., an ex vivo system for propagating cells or producing biological materials, as disclosed in co-pending U.S. application Ser. No. 10/004,942, filed Dec. 5, 2001, entitled “Method of Collecting Placental Stem Cells” and U.S. application Ser. No. 10/076,180, filed Feb. 13, 2002, entitled “Post-Partum Mammalian Placenta, Its Use and Placental Stem Cells Therefrom,” both of which are incorporated herein by reference in their entireties. The number of propagated cells or level of biological material produced in the placental bioreactor is maintained in a continuous state of balanced growth by periodically or continuously removing a portion of a culture medium or perfusion fluid that is introduced into the placental bioreactor, and from which the propagated cells or the produced biological materials may be recovered. Fresh medium or perfusion fluid is introduced at the same rate or in the same amount.

The number and type of cells propagated may easily be monitored by measuring changes in morphology and cell surface markers using standard cell detection techniques such as flow cytometry, cell sorting, immunocytochemistry (e.g., staining with tissue specific or cell-marker specific antibodies) fluorescence activated cell sorting (FACS), magnetic activated cell sorting (MACS), by examination of the morphology of cells using light or confocal microscopy, or by measuring changes in gene expression using techniques well known in the art, such as PCR and gene expression profiling. For methods of monitoring numbers and types of cells and for cell separation, see co-pending U.S. application Ser. No. 10/004,942, filed Dec. 5, 2001, entitled “Method of Collecting Placental Stem Cells” and U.S. application Ser. No. 10/076,180, filed Feb. 13, 2002, entitled “Post-Partum Mammalian Placenta, Its Use and Placental Stem Cells Therefrom.”

In preferred embodiments, the placenta to be used as a bioreactor is exsanguinated and washed under sterile conditions so that any adherent coagulated and non-adherent cellular contaminants are removed. The placenta is then cultured or cultivated under aseptic conditions as disclosed in co-pending application Ser. No. 10/004,942, filed Dec. 5, 2001, entitled “Method of Collecting Placental Stem Cells” and application Ser. No. 10/076,180, filed Feb. 13, 2002, entitled “Post-Partum Mammalian Placenta, Its Use and Placental Stem Cells Therefrom.”

In certain embodiments, the embryonic-like stem cells are induced to propagate in the placenta bioreactor by introduction of nutrients, hormones, vitamins, growth factors, or any combination thereof, into the perfusion solution. Serum and other growth factors may be added to the propagation perfusion solution or medium. Growth factors are usually proteins and include, but are not limited to: cytokines, lymphokines, interferons, colony stimulating factors (CSFs), interferons, chemokines, and interleukins. Other growth factors that may be used include recombinant human hematopoietic growth factors including ligands, stem cell factors, thrombopoeitin (Tpo), granulocyte colony-stimulating factor (G-CSF), leukemia inhibitory factor, basic fibroblast growth factor, placenta derived growth factor and epidermal growth factor.

The growth factors introduced into the perfusion solution can stimulate the propagation of undifferentiated embryonic-like stem cells, committed progenitor cells, or differentiated cells (e.g., differentiated hematopoictic cells). The growth factors can stimulate the production of biological materials and bioactive molecules including, but not limited to, immunoglobulins, hormones, enzymes or growth factors as previously described. The cultured placenta should be “fed” periodically to remove the spent media, depopulate released cells, and add fresh media. The cultured placenta should be stored under sterile conditions to reduce the possibility of contamination, and maintained under intermittent and periodic pressurization to create conditions that maintain an adequate supply of nutrients to the cells of the placenta. It should be recognized that the perfusing and culturing of the placenta can be both automated and computerized for efficiency and increased capacity.

In another embodiment, the placenta is processed to remove all endogenous proliferating cells, such as embryonic-like stem cells, and to allow foreign (i.e., exogenous) cells to be introduced and propagated in the environment of the perfused placenta. The invention contemplates a large variety of stem or progenitor cells that can be cultured in the placental bioreactor, including, but not limited to, embryonic-like stem cells, mesenchymal stem cells, stromal cells, endothelial cells, hepatocytes, keratinocytes, and stem or progenitor cells for a particular cell type, tissue or organ, including but not limited to neurons, myelin, muscle, blood, bone marrow, skin, heart, connective tissue, lung, kidney, liver, and pancreas (e.g., pancreatic islet cells).

In certain embodiments of the invention, stem or progenitor cells are cultivated or propagated within a postpartum perfused placenta wherein they are exposed, according to the methods of the invention, to compounds that modulate the differentiation of the cultivated cells. Examples of the small molecule compounds that may be used include, but are not limited to, compounds that inhibit JNK or MKK activity. In one embodiment, the compound is not a polypeptide, peptide, protein, hormone, cytokine, oligonucleotide, or nucleic acid, as described above. The placental mesoderm provides an ideal stromal environment, including an abundance of small molecules and growth factors, lipopolysaccharides, and extracellular matrix proteins, necessary for organogenesis and tissue neogenesis.

In one embodiment, the invention provides a method of utilizing the isolated perfused placenta as a bioreactor for the propagation of exogenous cells. In accordance with this embodiment, the invention relates to an isolated placenta which contains a cell not derived from the placenta, wherein the engraftment of said cell into the placenta may stimulate the placenta to produce embryonic-like stem cells, or wherein the engrafted cell produces signals, such as cytokines and growth factors, which may stimulate the placenta to produce stem cells. The placenta may be engrafted with cells not placental in origin obtained from the parents, siblings or other blood relatives of the infant associated with the placenta.

In another embodiment, the isolated placenta may be engrafted with cells not placental in origin obtained from an individual that is not the infant associated with the placenta, nor related to the infant. Likewise, the cells, tissues, organoids and organs, which are propagated and cultivated in the placenta may be transplanted into the infant associated with the placenta, the parents, siblings or other blood relatives of said infant or into an individual not related to the infant.

In one embodiment of the invention, the placenta can be populated with any particular cell type and used as a bioreactor for ex vivo cultivation of cells, tissues or organs. Such cells, tissue or organ cultures may be harvested used in transplantation and ex vivo treatment protocols. In this embodiment, the placenta is processed to remove all endogenous cells and to allow foreign (i.e., exogenous) cells to be introduced and propagated in the environment of the perfused placenta. Methods for removal of the endogenous cells are well-known in the art. For example, the perfused placenta is irradiated with electromagnetic, UV, X-ray, gamma- or beta-radiation to eradicate all remaining viable, endogenous cells. In one embodiment, sub-lethal exposure to radiations e.g., 500 to 1500 CGγ can be used to preserve the placenta but eradicate undesired cells. For international on lethal v. non-lethal ionizing radiation (see Chapter 5 “Biophysical and Biological Effects of Ionizing Radiation” from the United States Department of Defense The foreign cells of interest to be propagated in the irradiated placental bioreactor are then introduced, for example, by vascular perfusion or direct intra-parenchymal injection.

In another embodiment, the bioreactor may be used to produce and propagate novel chimeric cells, tissues, or organs. Such chimeras may be created using placental cells and one or more additional cell types as starting materials in a bioreactor. The interaction, or “cross-talk” between the different cell types can induce expression patterns distinct from either of the starting cell types. In one embodiment, for example, an autologous chimera is generated by propagating a patient's autologous placental cells in a bioreactor with another cell type derived from the same patient. In another embodiment, for example, a heterologous chimera may be generated by addition of a patient's cells, i.e., blood cells, to a bioreactor having heterologous placental cells. In yet another embodiment, the placental cells may be derived from a patient, and a second cell type from a second patient. Chimeric cells are then recovered having a different phenotypic and/or genetic characteristics from either of the starting cells. In a specific embodiment, the heterologous cells are of the same haplotype, and the chimeric cells are reintroduced into the patient.

In other embodiments, the bioreactor may be used for enhanced growth of a particular cell type, whether native or synthetic in origin, or for the production of a cell-type specific product. For example, in one embodiment, the placental bioreactor may be used to stimulate pancreatic islet cells to produce insulin. The bioreactor is particularly advantageous for production of therapeutic mammalian proteins, whose therapeutic efficacy can be dependent upon proper post-translational modification. Thus, the bioreactor is useful for the production of therapeutic proteins, antibodies, growth factors, cytokines, and other natural or recombinant therapeutic molecules, such as but not limited to, erythropoietin, interleukins, and interferons.

In certain embodiments, a specific population of stem cells or progenitor cells is conditioned for differentiation within the placental bioreactor. In other embodiments, a specific population of stem cells or progenitor cells is induced to differentiate within the placental bioreactor. Such specific populations of stem or progenitor cells include, but are not limited to embryonic-like stem cells, embryonic stem cells, pluripotent cells, multipotent cells, totipotent cells, and committed progenitor cells (e.g., chondrocytes, hepatocytes, hematopoietic cells, pancreatic parenchymal cells, neuroblasts, muscle progenitor cells, etc.). In such embodiments, the compounds of the invention may be introduced into the placental bioreactor, e.g., via perfusion, and used according to the methods of the invention to condition stem cell or progenitor cell differentiation. For example, in a specific embodiment, exogenous CD34− stem cells or progenitor cells are cultivated within the placental bioreactor, and exposed to a JNK or MKK inhibitor, wherein differentiation of CD34+ cells from CD34− is upregulated or enhanced.

In another embodiment of the invention, the placenta is used as a bioreactor for propagating endogenous cells (i.e., cells that originate from the placenta), including but not limited to, various kinds of pluripotent and/or totipotent embryonic-like stem cells and lymphocytes. In one embodiment, the placenta is incubated for varying periods of time with perfusate solution as disclosed herein. Such endogenous cells of placental origin may be transformed to recombinantly express a gene of interest, to express mutations, and/or may be engineered to delete a genetic locus, using “knock out” technology. For example, an endogenous target gene may be deleted by inactivating or “knocking out” the target gene or its promoter using targeted homologous recombination (e.g., see Smithies, et al., 1985, Nature 317, 230-234; Thomas & Capecchi, 1987, Cell 51, 503-512; Thompson, et al., 1989, Cell 5, 313-321; each of which is incorporated by reference herein in its entirety). For example, a mutant, non-functional target gene (or a completely unrelated DNA sequence) flanked by DNA homologous to the endogenous target gene (either the coding regions or regulatory regions of the target gene) can be used, with or without a selectable marker and/or a negative selectable marker, to transfect cells that express the target gene in vivo. Insertion of the DNA construct, via targeted homologous recombination, results in inactivation of the target gene. Such approaches may be used to remove, replace, or alter gene expression of interest in cells, tissue, and/or organs. This approach may be used to alter the phenotype of a cell, tissue, or organ, which may then be introduced into a human subject.

In other embodiments, a placenta cell may be induced to differentiate into a particular cell type, either ex vivo or in vivo. For example, pluripotent embryonic-like stem cells may be injected into a damaged organ, and for organ neogenesis and repair of injury in vivo. Such injury may be due to such conditions and disorders including, but not limited to, myocardial infarction, seizure disorder, multiple sclerosis, stroke, hypotension, cardiac arrest, ischemia, inflammation, age-related loss of cognitive function, radiation damage, cerebral palsy, neurodegenerative disease, Alzheimer's disease, Parkinson's disease, Leigh disease, AIDS dementia, memory loss, amyotrophic lateral sclerosis, ischemic renal disease, brain or spinal cord trauma, heart-lung bypass, glaucoma, retinal ischemia, or retinal trauma.

The embryonic-like stem cells isolated from the placenta may be used, in specific embodiments, in autologous or heterologous enzyme replacement therapy to treat specific diseases or conditions, including, but not limited to lysosomal storage diseases, such as Tay-Sachs, Niemann-Pick, Fabry's, Gaucher's, Hunter's, and Hurler's syndromes, as well as other gangliosidoses, mucopolysaccharidoses, and glycogenoses.

Transplanted treated bone marrow cells may be used to treat malignant disease (e.g., patients suffering from acute lymphocytic leukemia, acute myelogenous leukemia, chronic myelogenous leukemia, chronic lymphocytic leukemia, myelodysplastic syndrome (“preleukemia”), monosomy 7 syndrome, non-Hodgkin's lymphoma, neuroblastoma, brain tumors, multiple myeloma, testicular germ cell tumors, breast cancer, lung cancer, ovarian cancer, melanoma, glioma, sarcoma or other solid tumors) or a non-malignant disease (e.g., hematologic disorders, congenital immunodeficiences, mucopolysaccharidoses, lipidoses, osteoporosis, Langerhan's cell histiocytosis, Lesch-Nyhan syndrome or glycogen storage diseases).

In other embodiments, the cells may be used as autologous or heterologous transgene carriers in gene therapy to correct inborn errors of metabolism, adrenoleukodystrophy, cystic fibrosis, glycogen storage disease, hypothyroidism, sickle cell anemia, Pearson syndrome, Pompe's disease, phenylketonuria (PKU), porphyrias, maple syrup urine disease, homocystinuria, mucopolysaccharidenosis, chronic granulomatous disease and tyrosinemia and Tay-Sachs disease or to treat cancer, tumors or other pathological conditions.

In other embodiments, the cells may be used in autologous or heterologous tissue regeneration or replacement therapies or protocols, including, but not limited to treatment of corneal epithelial defects, cartilage repair, facial dermabrasion, mucosal membranes, tympanic membranes, intestinal linings, neurological structures (e.g., retina, auditory neurons in basilar membrane, olfactory neurons in olfactory epithelium), burn and wound repair for traumatic injuries of the skin, or for reconstruction of other damaged or diseased organs or tissues.

4.5. Genetic Engineering of Stem Cells

In another embodiment, stem or progenitor cells to be differentiated in accordance with the methods of the invention are genetically engineered either prior to, or after exposure to the compounds of the invention, using, for example, a viral vector such as an adenoviral or retroviral vector, or by using mechanical means such as liposomal or chemical mediated uptake of the DNA.

A vector containing a transgene can be introduced into a cell of interest by methods well known in the art, e.g., transfection, transformation, transduction, electroporation, infection, microinjection, cell fusion, DEAE dextran, calcium phosphate precipitation, liposomes, LIPOFECTIN™, lysosome fusion, synthetic cationic lipids, use of a gene gun or a DNA vector transporter, such that the transgene is transmitted to daughter cells, e.g., the daughter embryonic-like stem cells or progenitor cells produced by the division of an embryonic-like stem cell. For various techniques for transformation or transfection of mammalian cells, see Keown et al., 1990, Methods Enzymol. 185: 527-37; Sambrook et al., 2001, Molecular Cloning, A Laboratory Manual, Third Edition, Cold Spring Harbor Laboratory Press, N.Y.

Preferably, the transgene is introduced using any technique, so long as it is not destructive to the cell's nuclear membrane or other existing cellular or genetic structures. In certain embodiments, the transgene is inserted into the nucleic genetic material by microinjection. Microinjection of cells and cellular structures is commonly known and practiced in the art.

For stable transfection of cultured mammalian cells, such as cells culture in a placenta, only a small fraction of cells may integrate the foreign DNA into their genome. The efficiency of integration depends upon the vector and transfection technique used. In order to identify and select integrants, a gene that encodes a selectable marker (e.g., for resistance to antibiotics) is generally introduced into the host embryonic-like stem cell along with the gene sequence of interest. Preferred selectable markers include those that confer resistance to drugs, such as G418, hygromycin and methotrexate. Cells stably transfected with the introduced nucleic acid can be identified by drug selection (e.g., cells that have incorporated the selectable marker gene will survive, while the other cells die). Such methods are particularly useful in methods involving homologous recombination in mammalian cells (e.g., in embryonic-like stem cells) prior to introduction or transplantation of the recombinant cells into a subject or patient.

A number of selection systems may be used to select transformed host embryonic-like cells. In particular, the vector may contain certain detectable or selectable markers. Other methods of selection include but are not limited to selecting for another marker such as: the herpes simplex virus thymidine kinase (Wigler et al., 1977, Cell 11: 223), hypoxanthine-guanine phosphoribosyltransferase (Szybalska and Szybalski, 1962, Proc. Natl. Acad. Sci. USA 48: 2026), and adenine phosphoribosyltransferase (Lowy et al., 1980, Cell 22: 817) genes can be employed in tk-, hgprt- or aprt-cells, respectively. Also, antimetabolite resistance can be used as the basis of selection for the following genes: dhfr, which confers resistance to methotrexate (Wigler et al., 1980, Proc. Natl. Acad. Sci. USA 77: 3567; O'Hare et al., 1981, Proc. Natl. Acad. Sci. USA 78: 1527); gpt, which confers resistance to mycophenolic acid (Mulligan and Berg, 1981, Proc. Natl. Acad. Sci. USA 78: 2072); neo, which confers resistance to the aminoglycoside G-418 (Colberre-Garapin et al., 1981, J. Mol. Biol. 150: 1); and hygro, which confers resistance to hygromycin (Santerre et al., 1984, Gene 30: 147).

The transgene may integrate into the genome of the cell of interest, preferably by random integration. In other embodiments the transgene may integrate by a directed method, e.g., by directed homologous recombination (i.e., “knock-in” or “knock-out” of a gene of interest in the genome of cell of interest), Chappel, U.S. Pat. No. 5,272,071; and PCT publication No. WO 91/06667, published May 16, 1991; U.S. Pat. No. 5,464,764; Capecchi et al., issued Nov. 7, 1995; U.S. Pat. No. 5,627,059, Capecchi et al. issued, May 6, 1997; U.S. Pat. No. 5,487,992, Capecchi et al., issued Jan. 30, 1996).

Methods for generating cells having targeted gene modifications through homologous recombination are known in the art. The construct will comprise at least a portion of a gene of interest with a desired genetic modification, and will include regions of homology to the target locus, i.e., the endogenous copy of the targeted gene in the host's genome. DNA constructs for random integration, in contrast to those used for homologous recombination, need not include regions of homology to mediate recombination. Markers can be included in the targeting construct or random construct for performing positive and negative selection for insertion of the transgene.

To create a homologous recombinant cell, e.g., a homologous recombinant embryonic-like stem cell, endogenous placental cell or exogenous cell cultured in the placenta, a homologous recombination vector is prepared in which a gene of interest is flanked at its 5′ and 3′ ends by gene sequences that are endogenous to the genome of the targeted cell, to allow for homologous recombination to occur between the gene of interest carried by the vector and the endogenous gene in the genome of the targeted cell. The additional flanking nucleic acid sequences are of sufficient length for successful homologous recombination with the endogenous gene in the genome of the targeted cell. Typically, several kilobases of flanking DNA (both at the 5′ and 3′ ends) are included in the vector. Methods for constructing homologous recombination vectors and homologous recombinant animals from recombinant stem cells are commonly known in the art (see, e.g., Thomas and Capecchi, 1987, Cell 51: 503; Bradley, 1991, Curr. Opin. Bio/Technol. 2: 823-29; and PCT Publication Nos. WO 90/11354, WO 91/01140, and WO 93/04169.

In one embodiment, the genome of an exogenous cell cultured in the placenta according to the methods of the invention is a target of gene targeting via homologous recombination or via random integration.

In a specific embodiment, the methods of Bonadio et al. (U.S. Pat. No. 5,942,496, entitled Methods and compositions for multiple gene transfer into bone cells, issued Aug. 24, 1999; and PCT WO95/22611, entitled Methods and compositions for stimulating bone cells, published Aug. 24, 1995) are used to introduce nucleic acids into a cell of interest, such as a stem cell, progenitor cell or exogenous cell cultured in the placenta, e.g., bone progenitor cells.

4.6. Uses of Stem Cells Conditioned for Differentiation

The stem cells of the invention may be induced to differentiate for use in transplantation and ex vivo treatment protocols. In one embodiment, the stem cell populations are differentiated to a particular cell type and genetically engineered to provide a therapeutic gene product.

The compounds of the invention also have utility in clinical settings in which transplantation has the principle objective of restoring bone marrow white blood cell production, such as the reversal of neutropenia and leukopenia, which result from disease and/or clinical myeloablation. The compounds of the invention also have utility in cases in which the suppression of red blood cell generation is preferred, without bone marrow suppression.

In certain embodiments, stem cells that have been treated with the compounds of the invention are administered along with untreated cells, such as stem cells from cord blood or peripheral blood, to a patient in need thereof.

Stem cells, e.g., embryonic-like stem cells, the differentiation of which has been modulated according to the methods of the invention, may be formulated as an injectable (see PCT WO 96/39101, incorporated herein by reference in its entirety). In an alternative embodiment, cells and tissues, the differentiation of which has been modulated according to the methods of the invention, may be formulated using polymerizable or cross linking hydrogels as described in U.S. Pat. Nos. 5,709,854; 5,516,532; 5,654,381; each of which is incorporated by reference in its entirety.

The embryonic-like stem cells, the differentiation of which has been modulated according to the methods of the invention, can be used for a wide variety of therapeutic protocols in which a tissue or organ of the body is augmented, repaired or replaced by the engraftment, transplantation or infusion of a desired cell population, such as a stem cell or progenitor cell population. The embryonic-like stem cells can be used to replace or augment existing tissues, to introduce new or altered tissues, or to join together biological tissues or structures. The embryonic-like stem cells can also be substituted for embryonic stem cells in therapeutic protocols in which embryonic stem cells would be typically be used.

In a preferred embodiment of the invention, embryonic-like stem cells and other stem cells from the placenta, the differentiation of which has been modulated according to the methods of the invention, may be used as autologous and allogenic, including matched and mismatched HLA type, hematopoietic transplants. In accordance with the use of embryonic-like stem cells as allogenic hematopoietic transplants, it may be preferable to treat the host to reduce immunological rejection of the donor cells, such as those described in U.S. Pat. No. 5,800,539, issued Sep. 1, 1998; and U.S. Pat. No. 5,806,529, issued Sep. 15, 1998, both of which are incorporated herein by reference.

For example, embryonic-like stem cells, the differentiation of which has been modulated according to the methods of the invention, can be used in therapeutic transplantation protocols, e.g., to augment or replace stem or progenitor cells of the liver, blood, lymphatic system, pancreas, kidney, lung, nervous system, muscular system, bone, bone marrow, thymus, spleen, mucosal tissue, gonads, or hair.

Embryonic-like stem cells may be used instead of specific classes of progenitor cells (e.g., chondrocytes, hepatocytes, hematopoietic cells, pancreatic parenchymal cells, neuroblasts, muscle progenitor cells, etc.) in therapeutic or research protocols in which progenitor cells would typically be used.

Embryonic-like stem cells of the invention can be used for augmentation, repair or replacement of cartilage, tendon, or ligaments. For example, in certain embodiments, prostheses (e.g., hip prostheses) are coated with replacement cartilage tissue constructs grown from embryonic-like stem cells of the invention. In other embodiments, joints (e.g., knee) are reconstructed with cartilage tissue constructs grown from embryonic-like stem cells. Cartilage tissue constructs can also be employed in major reconstructive surgery for different types of joints (for protocols, see e.g., Resnick, D., and Niwayama, G., eds., 1988, Diagnosis of Bone and Joint Disorders, 2d ed., W. B. Saunders Co.).

The embryonic-like stem cells of the invention can be used to repair damage of tissues and organs resulting from disease. In such an embodiment, a patient can be administered embryonic-like stem cells to regenerate or restore tissues or organs which have been damaged as a consequence of disease, e.g., enhance immune system following chemotherapy or radiation, repair heart tissue following myocardial infarction.

The embryonic-like stem cells of the invention can be used to augment or replace bone marrow cells in bone marrow transplantation. Human autologous and allogenic bone marrow transplantation are currently used as therapies for diseases such as leukemia, lymphoma and other life-threatening disorders. The drawback of these procedures, however, is that a large amount of donor bone marrow must be removed to insure that there is enough cells for engraftment.

The embryonic-like stem cells collected according to the methods of the invention can provide stem cells and progenitor cells that can be differentiated according to the methods of the invention, thus reducing the need for large bone marrow donation. The methods of the invention also include obtaining a small marrow donation and then expanding the number of stem cells and progenitor cells, for example, by culturing and expanding in a placenta, before infusion or transplantation into a recipient.

The embryonic-like stem cells isolated from the placenta, contacted with one or more of the compounds of the invention, may be used, in specific embodiments, in autologous or heterologous enzyme replacement therapy to treat specific diseases or conditions, including, but not limited to lysosomal storage diseases, such as Tay-Sachs, Niemann-Pick, Fabry's, Gaucher's, Hunter's, Hurler's syndromes, as well as other gangliosidoses, mucopolysaccharidoses, and glycogenoses.

In other embodiments, the cells may be used as autologous or heterologous transgene carriers in gene therapy to correct inborn errors of metabolism such as adrenoleukodystrophy, cystic fibrosis, glycogen storage disease, hypothyroidism, sickle cell anemia, Pearson syndrome, Pompe's disease, phenylketonuria (PKU), and Tay-Sachs disease, porphyrias, maple syrup urine disease, homocystinuria, mucopolysaccharidenosis, chronic granulomatous disease, and tyrosinemia, or to treat cancer, tumors or other pathological conditions.

In other embodiments, the cells may be used in autologous or heterologous tissue regeneration or replacement therapies or protocols, including, but not limited to treatment of corneal epithelial defects, cartilage repair, facial dermabrasion, mucosal membranes, tympanic membranes, intestinal linings, neurological structures (e.g., retina, auditory neurons in basilar membrane, olfactory neurons in olfactory epithelium), burn and wound repair for traumatic injuries of the skin, scalp (hair) transplantation, or for reconstruction of other damaged or diseased organs or tissues.

The large numbers of embryonic-like stem cells and/or progenitor obtained using the methods of the invention would, in certain embodiments, reduce the need for large bone marrow donations. Approximately 1×10⁸ to 2×10⁸ bone marrow mononuclear cells per kilogram of patient weight must be infused for engraftment in a bone marrow transplantation (i.e., about 70 ml of marrow for a 70 kg donor). To obtain 70 ml requires an intensive donation and significant loss of blood in the donation process. In a specific embodiment, cells from a small bone marrow donation (e.g., 7-10 ml) could be expanded by propagation in a placental bioreactor before infusion into a recipient.

Furthermore, a small number of stem cells and progenitor cells normally circulate in the blood stream. In another embodiment, such exogenous stem cells or exogenous progenitor cells are collected by apheresis, a procedure in which blood is withdrawn, one or more components are selectively removed, and the remainder of the blood is reinfused into the donor. The exogenous cells recovered by apheresis are expanded by the methods of the invention, thus eliminating the need for bone marrow donation entirely.

In another embodiment, expansion of hematopoietic progenitor cells in accordance with the methods of the invention is used as a supplemental treatment in addition to chemotherapy. Most chemotherapy agents used to target and destroy cancer cells act by killing all proliferating cells, i.e., cells going through cell division. Since bone marrow is one of the most actively proliferating tissues in the body, hematopoietic stem cells are frequently damaged or destroyed by chemotherapy agents and in consequence, blood cell production is diminishes or ceases. Chemotherapy must be terminated at intervals to allow the patient's hematopoietic system to replenish the blood cell supply before resuming chemotherapy. It may take a month or more for the formerly quiescent stem cells to proliferate and increase the white blood cell count to acceptable levels so that chemotherapy may resume (when again, the bone marrow stem cells are destroyed).

While the blood cells regenerate between chemotherapy treatments, however, the cancer has time to grow and possibly become more resistant to the chemotherapy drugs due to natural selection. Therefore, the longer chemotherapy is given and the shorter the duration between treatments, the greater the odds of successfully killing the cancer. To shorten the time between chemotherapy treatments, embryonic-like stem cells or progenitor cells differentiated in accordance with the methods of the invention could be introduced into the patient. Such treatment would reduce the time the patient would exhibit a low blood cell count, and would therefore permit earlier resumption of the chemotherapy treatment.

In another embodiment, the human placental stem cells can be used to treat or prevent genetic diseases such as chronic granulomatous disease.

4.7. Methods for Treating or Preventing MPD or MDS

The invention encompasses methods for treating or preventing MPD comprising administering to a patient in need thereof an effective amount of a JNK or MKK inhibitor. The MPD can be primary or secondary. The invention further encompasses methods for treating patients who have been previously treated for MPD, as well as those who have not previously been treated for MPD. Because patients with MPD have heterogenous clinical manifestations and varying clinical outcomes, staging the patients according to their prognosis and approaching therapy depending on the severity and stage may be necessary. Indeed, the types of MPD treatable or preventable according to the methods of the invention include, but are not limited to, polycythemia rubra vera (PRV); primary thrombocythemia (PT); chronic myelogenous leukemia (CML); acute or chronic granulocytic leukemia; acute or chronic myelomonocytic leukemia; myelofibro-erythroleukemia; and agnogenic myeloid metaplasia (AMM).

The invention encompasses treating or preventing MPD or one or more symptoms or abnormalities associated with MPD comprising administering to a patient in need thereof an effective amount of a JNK or MKK inhibitor. Such an inhibitor may be administered to the patient in any one of the forms provided in Section 4.8, below. Specifically, the patient may be administered the inhibitor alone, or may be administered an inhibitor in combination with a pharmaceutical composition comprising a stem cell, cord blood cell, progenitor cell, or cord blood stem or progenitor cell that has been contacted with a JNK or MKK modulator, such as a JNK or MKK inhibitor, for a sufficient time for modulation of JNK or MKK activity, or for modulation of differentiation or proliferation of the stem or progenitor cell. Pharmaceutical compositions comprising such cells may alternatively be administered to the patient without a JNK or MKK modulatory compound.

The invention further encompasses methods of treating or preventing MDS, comprising administering to a patient in need thereof an effective amount of a JNK or MKK inhibitor. The MDS can be primary or secondary. The invention further encompasses methods of treating patients who have been previously treated for MDS, as well as those who have not previously been treated for MDS. Because patients with MDS have heterogenous clinical manifestations and varying clinical outcomes, staging the patients according to their prognosis and approaching therapy depending on the severity and stage is necessary. The types of MDS treatable or preventable according to the methods of the invention include, but are not limited to, refractory anemia (RA), RA with ringed sideroblasts (RARS), RA with excess blasts (RAEB), RAEB in transformation (RAEB-T), preleukemia and chronic myelomonocytic leukemia (CMML).

The invention encompasses treating or preventing MDS or one or more symptoms or abnormalities associated with MDS comprising administering to a patient in need thereof an effective amount of a JNK or MKK inhibitor. Such an inhibitor may be administered to the patient in any one of the forms provided in Section 4.8, below. Specifically, the patient may be administered the inhibitor alone, or may be administered an inhibitor in combination with a pharmaceutical composition comprising cord blood, a cord blood cell, a cord blood stem or progenitor cell, a stem cell or a progenitor cell that has been contacted with a JNK or MKK modulator, such as a JNK or MKK inhibitor, for a sufficient time for modulation of JNK or MKK activity, or for modulation of differentiation or proliferation of the stem or progenitor cell. Pharmaceutical compositions comprising such cells may alternatively be administered to the patient without a JNK or MKK modulatory compound.

In one embodiment, the invention provides a method of treating or preventing a myeloproliferative disorder, comprising administering to a patient in need thereof an effective amount of a JNK inhibitor or an MKK inhibitor. In a specific embodiment, the myeloproliferative disorder is polycythemia rubra vera; primary thrombocythemia; chronic myclogenous leukemia; acute or chronic granulocytic leukemia; acute or chronic myelomonocytic leukemia; myelofibro-erythroleukemia; or agnogenic myeloid metaplasia. The invention also provides a method for treating or preventing a symptom of or an abnormality associated with a myeloproliferative disorder, comprising administering to a patient in need thereof an effective amount of a JNK inhibitor or an MKK inhibitor. In a specific embodiment, the symptom is headache, dizziness, tinnitus, blurred vision, fatigue, night sweat, low-grade fever, generalized pruritus, epistaxis, blurred vision, splenomegaly, abdominal fullness, thrombosis, increased bleeding, anemia, splenic infarction, severe bone pain, hematopoiesis in the liver, ascites, esophageal varices, liver failure, respiratory distress or priapism. In another specific embodiment, the abnormality is clonal expansion of a multipotent hematopoietic progenitor cell with the overproduction of one or more of the formed elements of the blood, presence of Philadelphia chromosome or bcr-abl gene, teardrop poikilocytosis on peripheral blood smear, leukoerythroblastic blood picture, giant abnormal platelets, hypercellular bone marrow with reticular or collagen fibrosis or marked left-shifted myeloid series with a low percentage of promyelocytes and blasts. The invention further provides a method for treating or preventing a myelodysplastic syndrome, comprising administering to a patient in need thereof an effective amount of a JNK inhibitor or an MKK inhibitor. In a specific embodiment, the myelodysplastic syndrome is refractory anemia, refractory anemia with ringed sideroblasts, refractory anemia with excess blasts, refractory anemia with excess blasts in transformation, preleukemia or chronic myelomonocytic leukemia. The invention also provides a method for treating or preventing a symptom of a myelodysplastic syndrome, comprising administering to a patient in need thereof an effective amount of a JNK inhibitor or an MKK inhibitor. In a specific embodiment, the symptom is anemia, thrombocytopenia, neutropenia, bicytopenia or pancytopenia.

4.8. Pharmaceutical Compositions

The JNK or MKK inhibitors useful in the present methods can be administered to a patient in the form of a pharmaceutical composition; in one embodiment, in a single unit dosage form. Such pharmaceutical compositions and unit dosage forms comprise an effective amount of a JNK or MKK inhibitor and a pharmaceutically acceptable carrier or vehicle.

Single unit dosage forms of the invention are suitable for oral, mucosal (e.g., nasal, sublingual, vaginal, buccal, or rectal), or parenteral (e.g., subcutaneous, intravenous, bolus injection, intramuscular, or intraarterial), transdermal, intravitreal or transcutaneous administration to a patient.

Examples of single unit dosage forms include, but are not limited to: tablets; caplets; capsules, such as soft elastic gelatin capsules; cachets; troches; lozenges; dispersions; suppositories; powders; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage form can vary depending on their use. For example, a dosage form used in the acute treatment of a disease can comprise greater amounts of a JNK or MKK inhibitor relative to a dosage form used in the chronic treatment of the same disease. Similarly, a parenteral dosage form can comprise smaller amounts of a JNK or MKK inhibitor relative to an oral dosage form used to treat the same disease. These and other ways in which specific dosage forms encompassed by this invention will vary from one another are readily apparent to those skilled in the art. See, e.g., Remington's Pharmaceutical Sciences (18th ed.1990).

In another embodiment, the invention encompasses pharmaceutical compositions comprising isolated cord blood populations which have been augmented with hematopoietic progenitor cells which have been differentiated by exposure to an effective amount of a JNK or MKK inhibitor, in accordance with the methods of the invention.

In another embodiment, the invention encompasses pharmaceutical compositions comprising untreated hematopoietic progenitor cells in combination with an effective amount of a JNK or MKK inhibitor, in accordance with the methods of the invention.

Typical pharmaceutical compositions and dosage forms comprise a pharmaceutically acceptable carrier or vehicle. Pharmaceutical compositions and dosage forms can further comprise a pharmaceutically acceptable excipient. Suitable excipients are well known to those skilled in the art of pharmacy, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets can contain excipients not suited for use in parenteral dosage forms. The suitability of a particular excipient may also depend on the specific active agents in the dosage form. For example, the decomposition of some active agents may be accelerated by some excipients such as lactose, or when exposed to water. Consequently, this invention encompasses pharmaceutical compositions and dosage forms that contain little, if any, lactose other mono- or di-saccharides. As used herein, the term “lactose-free” means that the amount of lactose present, if any, is insufficient to substantially increase the degradation rate of an active agent.

Lactose-free compositions of the invention can comprise excipients that are well known in the art and are listed, for example, in the U.S. Pharmacopeia (USP) 25-NF20 (2002). In general, lactose-free compositions comprise a JNK or MKK inhibitor, a binder or filler, and a lubricant in pharmaceutically compatible and pharmaceutically acceptable amounts. In one embodiment, lactose-free dosage forms comprise a JNK or MKK inhibitor, microcrystalline cellulose, pre-gelatinized starch, and magnesium stearate.

Anhydrous (comprising less than about 5% water by weight) pharmaceutical compositions and dosage forms of the invention can be prepared using anhydrous or low-moisture containing agents and low-moisture or low-humidity conditions.

An anhydrous pharmaceutical composition can be prepared and stored such that its anhydrous nature is maintained. Accordingly, anhydrous compositions can be packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further encompasses pharmaceutical compositions and dosage forms that comprise a stabilizer including, but not limited to, antioxidants such as ascorbic acid, pH buffers, or salt buffers.

4.8.1 Oral Dosage Forms

Pharmaceutical compositions of the invention that are suitable for oral administration can be presented as discrete dosage forms including, but not limited to, tablets (e.g., chewable tablets), caplets, capsules, and liquids (e.g., flavored syrups). Such dosage forms comprise a predetermined amount of a JNK or MKK inhibitor, and may be prepared by methods of pharmacy well known to those skilled in the art. See generally, Remington's Pharmaceutical Sciences, (18th ed. 1990).

Typical oral dosage forms of the invention are typically prepared by admixing a JNK or MKK inhibitor with a pharmaceutically acceptable carrier or vehicle according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by admixing the JNK or MKK inhibitor with liquid carriers, finely divided solid carriers, or both, and then shaping the product into the desired presentation if necessary.

For example, a tablet can be prepared by compression or molding. Compressed tablets can be prepared by compressing in a suitable machine the JNK or MKK inhibitor in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms of the invention include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, the materials sold as AVICEL-PH-101, AVICEL-PH-103, AVICEL RC-581, AVICEL-PH-105 (available from FMC Corporation, American Viscose Division, Avicel Sales, Marcus Hook, Pa.), and mixtures thereof. An specific binder is a mixture of microcrystalline cellulose and sodium carboxymethyl cellulose sold as AVICEL RC-581. Suitable anhydrous or low moisture excipients or additives include AVICEL-PH-103 and Starch 1500 LM.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form.

Disintegrants can be used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active agents should be used to form solid oral dosage forms of the invention. The amount of disintegrant used varies based upon the type of formulation, and is readily discernible to those skilled in the art. Pharmaceutical compositions can comprise from about 0.5 to about 15 weight percent of disintegrant, in one embodiment, from about 1 to about 5 weight percent of disintegrant.

Disintegrants that can be used in pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

Lubricants that can be used in pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W. R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

A solid oral dosage form of the invention can comprise a JNK or MKK inhibitor, anhydrous lactose, microcrystalline cellulose, polyvinylpyrrolidone, stearic acid, colloidal anhydrous silica, and gelatin.

4.8.2 Delayed-Release Dosage Forms

JNK or MKK inhibitors can be administered by controlled-release means or by delivery devices that are well known to those skilled in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Dosage forms can be used to provide slow or controlled-release of one or more JNK or MKK inhibitors using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those skilled in the art, including those described herein, can be readily selected for use with the JNK or MKK inhibitor. Thus, the invention encompasses single unit dosage forms suitable for oral administration such as, but not limited to, tablets, capsules, gelcaps, and caplets that are adapted for controlled-release.

Controlled-release pharmaceutical compositions can have a common goal of improving drug therapy over that achieved by their non-controlled counterparts. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Controlled-release formulations can be designed to initially release an amount of a JNK or MKK inhibitor that promptly produces the desired therapeutic effect, and gradually and continually release of other amounts of JNK or MKK inhibitor to maintain this level of therapeutic or prophylactic effect over an extended period of time. In order to maintain this constant level of JNK or MKK inhibitor in the body, the JNK or MKK inhibitor should be released from the dosage form at a rate that will replace the amount of JNK or MKK inhibitor being metabolized and excreted from the body. Controlled-release of a JNK or MKK inhibitor can be stimulated by various conditions including, but not limited to, pH, temperature, enzymes, water, or other physiological conditions or compounds.

4.8.3 Parenteral Dosage Forms

Parenteral dosage forms can be administered to patients by various routes including, but not limited to, subcutaneous, intravenous (including bolus injection), intramuscular, intravitreal and intraarterial. Because their administration typically bypasses patients' natural defenses against contaminants, parenteral dosage forms can be sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of a JNK or MKK inhibitor can also be incorporated into the parenteral dosage forms of the invention. For example, cyclodextrin and its derivatives can be used. See, e.g., U.S. Pat. No. 5,134,127, which is incorporated herein by reference.

4.8.4 Topical and Mucosal Dosage Forms

Topical and mucosal dosage forms of the invention include, but are not limited to, sprays, aerosols, solutions, emulsions, suspensions, or other forms known to one skilled in the art. See, e.g., Remington's Pharmaceutical Sciences (18^{th} ed.1990); and Introduction to Pharmaceutical Dosage Forms (4^{th} ed. 1985). Dosage forms suitable for treating mucosal tissues within the oral cavity can be formulated as mouthwashes or as oral gels.

Suitable excipients (e.g., carriers and diluents) and other materials that can be used to provide topical and mucosal dosage forms encompassed by this invention are well known to those skilled in the pharmaceutical arts, and can depend on the particular tissue to which a given pharmaceutical composition or dosage form will be applied. Typical excipients include, but are not limited to, water, acetone, ethanol, ethylene glycol, propylene glycol, butane-1,3-diol, isopropyl myristate, isopropyl palmitate, mineral oil, and mixtures thereof to form solutions, emulsions or gels, which are non-toxic and pharmaceutically acceptable. Moisturizers or humectants can also be added to pharmaceutical compositions and dosage forms if desired. Examples of such additional agents are well known in the art. See, e.g., Remington's Pharmaceutical Sciences (18^{th ed.} 1990).

The pH of a pharmaceutical composition or dosage form can also be adjusted to improve delivery of one or more active agents. Similarly, the polarity of a solvent carrier, its ionic strength, or tonicity can be adjusted to improve delivery. Compounds such as stearates can also be added to pharmaceutical compositions or dosage forms to alter the hydrophilicity or lipophilicity of a JNK or MKK inhibitor so as to improve delivery. In this regard, stearates can serve as a lipid vehicle for the formulation, as an emulsifying agent or surfactant, and as a delivery-enhancing or penetration-enhancing agent. Salts, hydrates or solvates of the JNK or MKK inhibitor can be used to further adjust the properties of the resulting composition.

In one embodiment of the invention, a JNK or MKK inhibitor is administered by a parenteral, intravenous, subcutaneous, intradermal, intravitreal, topical, mucosal or oral route and in a single or divided effective daily dose in an amount of from about 0.1 mg to about 2500 mg, from about 1 mg to about 2000 mg, or from 10 mg to about 1500 mg, or from 50 mg to about 1000 mg, or from 100 mg to about 750 mg, or from 250 mg to about 500 mg.

In one embodiment, the JNK or MKK inhibitor is administered to a patient as part of cycling therapy. Cycling therapy involves administration for a specified period of time, followed by administration for another specified period of time and repeating this sequential administration. Cycling therapy can reduce the development of resistance to one or more of the therapies, avoid or reduce the side effects of one of the therapies, and/or improve the efficacy of the treatment.

In one embodiment, a JNK or MKK inhibitor is administered in a cycle of about 16 weeks, about once or twice every day. One administration cycle can comprise the administration of a JNK inhibitor and at least one (1) or three (3) weeks of non-administration. The number of cycles can range from about 1 to about 12 cycles, more typically from about 2 to about 10 cycles, and more typically from about 2 to about 8 cycles.

The compounds of the invention are preferably assayed in vitro and in vivo, for the desired therapeutic or prophylactic activity, prior to use in humans. For example, in vitro assays can be used to determine whether administration of a specific compound of the invention or a combination of compounds of the invention is preferred. The compounds and compositions of the invention may also be demonstrated to be effective and safe using animal model systems. Other methods will be known to the skilled artisan and are within the scope of the invention.

The invention also provides pharmaceutical compositions that comprise a pharmaceutically acceptable carrier and stem cells and/or progenitor cells, wherein said cells have been contacted with a JNK or MKK modulator, preferably a JNK or MKK inhibitor, for a time effective to allow said modulation or inhibition of JNK or MKK activity. In one embodiment, these pharmaceutical compositions may comprise a single population of stem or progenitor cells that have been contacted with a JNK or MKK inhibitor, or multiple such populations, and may include such populations drawn entirely or partly, or not at all from the person who is the ultimate recipient of the composition. In another embodiment, the pharmaceutical compositions may comprise stem cells and/or progenitor cells contacted, or treated, with a JNK or MKK inhibitor, and untreated cells. In another embodiment, the pharmaceutical compositions may comprise treated stem cells and/or progenitor cells, in combination with peripheral blood or cord blood. In this embodiment, the peripheral blood or cord blood may be untreated, or may be treated separately from, or together with, the stem and/or progenitor cells. Any of the above pharmaceutical compositions may additionally comprise one or more JNK or MKK modulators, such as one or more JNK or MKK inhibitors.

Thus, in one embodiment, the invention provides a pharmaceutical composition comprising a mammalian stem cell and a pharmaceutically-acceptable carrier, wherein said stem cell has been contacted with a compound that inhibits JNK or MKK activity for a time sufficient to cause modulation of said JNK or MKK activity. In another embodiment, the invention provides a pharmaceutical composition comprising a mammalian progenitor cell and a pharmaceutically-acceptable carrier, wherein said progenitor cell has been contacted with a compound that inhibits JNK or MKK activity for a time sufficient to cause modulation of said JNK or MKK activity. In another embodiment, the invention provides a pharmaceutical composition comprising a mammalian stem cell or progenitor cell and a pharmaceutically-acceptable carrier, wherein said stem cell or progenitor cell has been contacted with a compound that inhibits JNK or MKK activity for a time sufficient to cause modulation of differentiation or proliferation of said stem cell or progenitor cell.

In specific embodiment, the invention provides that, for the above stem cell-containing pharmaceutical compositions, the stem cell is selected from the group consisting of an embryonic stem cell, an adult cell, a cord blood cell, a placental stem cell or a peripheral blood stem cell. In another specific embodiment of any of the above methods, the compound is an imide or amide. In another specific embodiment of any of the above methods, the contacting step is conducted in cell culture. In another specific embodiment, the concentration of the compound is from about 0.005 μg/ml to about 5 mg/ml. In another specific embodiment, the concentration of the compound is from about 1 μg/ml to about 2 mg/ml. In another specific embodiment, the stem cell is a human stem cell. In another specific embodiment of the progenitor cell-containing pharmaceutical compositions, said progenitor cell is a human progenitor cell. In another specific embodiment, the progenitor cell is a hematopoietic progenitor cell. In another specific embodiment of the above methods, said differentiation is differentiation into a hematopoictic cell. In more specific embodiment, the hematopoietic cell is CD34+ or CD38+. In another more specific embodiment, the hematopoietic cell is CD11b+.

The invention also provides a pharmaceutical composition comprising in a pharmaceutically acceptable carrier isolated cord blood cells and an isolated population of white blood cells, wherein the white blood cells are generated by a method comprising differentiating a stem cell under suitable conditions and in the presence of a compound that inhibits JNK activity or MKK activity, and isolating the white blood cells differentiated thereby. In another embodiment, the invention provides a pharmaceutical composition comprising isolated cord blood cells and an isolated population of white blood cells, wherein the white blood cells are generated by a method comprising differentiating a progenitor cell under suitable conditions and in the presence of a compound that inhibits JNK activity or MKK activity, and isolating the white blood cells differentiated thereby. In a specific embodiment, said differentiating is conducted in cell culture. In another specific embodiment, the concentration of the compound is between 0.005 μg/ml and 5 mg/ml. In another specific embodiment, the concentration of the compound is between 1 μg/ml and 2 mg/ml. In another specific embodiment, the stem cell is a human stem cell. In another specific embodiment, the progenitor cell is a hematopoietic progenitor cell.

The invention further provides preparations of differentiated cells, which may be included in the pharmaceutical compositions above, where appropriate, or may be used for different purposes. Such preparations may comprise stem cells or progenitor cells, preferably human stem cells or progenitor cells, that have been contacted with one or more JNK or MKK modulatory compounds for a time sufficient to detectably modulate, preferably to detectably inhibit, JNK or MKK activity within said cells, or for a time sufficient to modulate the proliferation or differentiation of said stem or progenitor cells. In a preferred embodiment, said stem or progenitor cells are contacted with one or more JNK or MKK modulators, preferably JNK or MKK inhibitors, for a time sufficient to differentiate said stem cell or progenitor cell to a terminally-differentiated cell. In a variation of the preparation, the preparation comprises stem cells that have been contacted with a JNK or MKK modulator for a sufficient time to differentiate the stem cells into progenitor cells of one or more cell lineages.

### 5. EXAMPLES

### 5.1. Example 1

### Effects of JNK or MKK Inhibitors on Differentiation of CD34+ Progenitor Cells

The following assay is utilized to determine the effects of JNK or MKK inhibitors on the differentiation of CD34+ (hematopoietic progenitor) cells and the generation of colony forming units (CFU). Significantly, the assay demonstrates the ability of JNK or MKK inhibitors to suppress specifically the generation of erythropoietic colonies (BFU-E and CFU-E), while augmenting both the generation of leukocyte and platelet forming colonies (CFU-GM) and enhancing total colony forming unit (CFU-Total) production. The methods of the invention can therefore be used to regulate the differentiation of stem cells, and can also be used to stimulate the rate of colony formation, providing significant benefits to hematopoietic stem cell transplantation by improving the speed of bone marrow engraftment and recovery of leukocyte and/or platelet production.

Cord blood CD34+ hematopoietic progenitor cells are plated in 96 well cultivation dishes at a density of 1000 cells per well in IMDM supplemented with 20% fetal calf serum and cytokines (IL-3, G-CSF and kit-ligand (R&D Systems, Inc.). The cells are exposed to JNK or MKK inhibitors at a concentration of between 0.001 μg/ml and 5 mg/ml, or DMSO (a control compound), and allowed to culture for 6 days. Cord blood CD34+ cells are plated in 96 well cultivation dishes at a density of 1000 cells per well in IMDM supplemented with 20% fetal calf serum and cytokines (IL-3, G-CSF and kit-ligand (KL) (R&D Systems, Inc.)). After culturing, cells are stained and sorted with a fluorescence activated cell sorter (FACS). 400 μL of stained cells are harvested and diluted to 1.0 ml with 1% fetal calf serum in phosphate buffered saline (PBS). Cells are counted to determine the effect of modulation of stem cell differentiation.

These results demonstrate that the compounds of the invention are effective in the modulation of the lineage commitment of hematopoietic progenitor stem cells. Thus, the compounds can be used to suppress specifically the generation of red blood cells or erythropoietic colonies (BFU-E and CFU-E), while augmenting both the generation of leukocyte and platelet forming colonies (CFU-GM) and enhancing total colony forming unit production. The methods of the invention can therefore be used to regulate the differentiation of stem cells, and can also be used to stimulate the rate of specific colony formation, providing significant benefits to hematopoietic stem cell transplantation by improving the speed of bone marrow engraftment and recovery of leukocyte and/or platelet production by origin stem cell commitment toward desired engraftable lineages.

### 5.2. Example 2

### Effects of JNK or MKK Inhibitors on Proliferation and Differentiation of Human Cord Blood CD34+ Cells

In the following example, the effects of JNK or MKK inhibitors on the proliferation and differentiation of cord blood (CB) mononuclear cells into CD34+ (hematopoietic progenitor) cells is studied. Cord blood mononuclear cells are a mixed population of cells including a small population of hematopoietic progenitor (CD34+) cells. A subset of this small CD34+ cell population includes a population (approximately 1% of total CB mononuclear cells) of CD34+CD38+cells and an even smaller population (less than 1% of total CB mononuclear cells) of CD34+CD38− cells. Significantly, the results can demonstrate an up-regulation (increased differentiation) of CD34+ cells, and inhibition or slowing down of the differentiation of hematopoietic stem cells or progenitor cells compared with positive and negative controls.

Materials and Methods: CB CD34+ cells are initiated at 4×10⁴ cells/ml in a 24-well plate in 20% FCS IMDM (fetal calf serum/Iscove's Modified Dulbecco's Medium) supplemented with cytokines (IL3, G-CSF and Kit-ligand) (R&D Systems, Inc.). JNK or MKK inhibitors are included in the culture at various concentrations. The same volumes of DMSO are used as controls. A negative control without any compound is also used. Cells are cultured at 37° C. and 5% CO₂ in a humidified incubator for 7 days. Cells are then harvested from each well.

The total cell number from each well is determined by counting in a CELL-DYN® 1700 (Abbott Diagnostics) and the expression of CXCR4, CD45, CD34, CD38, CD11b and Gly-A is analyzed by FACS (fluorescence-activated cell sorting) staining.

### 5.3. Example 3

### Effects of JNK or MKK Inhibitors on Human Cord Blood Mononuclear Cells

Cord blood MNCs that have been cryopreserved and thawed using standard methods are isolated by standard Ficoll separation method and cultured in 24 well-plate at 0.5×10⁶ cells/ml in 20% FCS-IMDM with cytokines (IL6, KL and G-CSF 10 ng/ml each) in triplicate. The experimental groups are None (cytokines only), DMSO (1.7 μL), and varying concentrations of a JNK or MKK inhibitor in DMSO. The cultured cells are harvested and analyzed by FACS staining after 1 week of culture.

### 5.4. Example 4

### Effects of JNK or MKK Inhibitors on Monocyte Production

Purified human cord blood CD34+ cells (greater than 90% CD34+) are cultured in 20% FCS IMDM medium supplemented with cytokines (IL3, IL6, G-CSF, KL and Epo) at 4×10⁴ cells/ml for 14 days at 37° C. in a humidified 5% CO₂ incubator. The experimental groups consist of a group in which (i) no DMSO or chemical compounds were added (“None”), (ii) DMSO only, and (iii) a JNK or MKK inhibitor dissolved in DMSO. Aliquots of cells are harvested and the expression of CD34 and CD14 is determined by staining with CD34-PE conjugated monoclonal antibody and CD14-FITC conjugated monoclonal antibody.

### 5.5. Example 5

### Effects of JNK or MKK Inhibitors on Transplanted Nucleated Cells from Umbilical Cord Blood and Placenta

This experiment demonstrates that JNK or MKK inhibitor pre-treatment increases the survival of transplanted placental nucleated cells (PLNC), umbilical cord blood nucleated cells (UCBNC) and bone marrow cells (BMNC).

Placental nucleated cells (PLNC), umbilical cord blood nucleated cells (UCBNC) and bone marrow cells (BMNC) are obtained from human donors. PLNC and UCBNC are obtained from placenta and umbilical cord using methods described in Section 4.1 and 4.2 above.

The cells are pretreated by incubating them in DMEM supplemented with 2% human CB serum with 10 μg/ml of a JNK or MKK inhibitor for 24 hours. Cells are then washed, resuspended in autologous plasma and administered intravenously to recipient adult SJL/L mice (Jackson Laboratories) that have had bone marrow ablation produced by lethal irradiation (900cGy) according to standard methods. Such irradiation is better than 90% lethal by 50 days post-irradiation (Ende et al., 2001, Life Sci. 69(13):1531-1539; Chen and Ende, 2000, J. Med. 31: 21-30; Ende et al., 2000, Life Sci. 67(1):53-9; Ende and Chen, 2000, Am. J. Clin. Pathol. 114: 89).

### 5.6. Example 6

### Induction of Differentiation into Particular Cell Types

Cord blood cells and/or embryonic-like stem cells are induced to differentiate into a particular cell type by exposure to a growth factor. Growth factors that are used to induce induction include, but are not limited to: GM-CSF, IL-4, Flt3L, CD40L, IFN-alpha, TNF-alpha, IFN-gamma, IL-2, IL-6, retinoic acid, basic fibroblast growth factor, TGF-beta-1, TGF-beta-3, hepatocyte growth factor, epidermal growth factor, cardiotropin-1, angiotensinogen, angiotensin I (AI), angiotensin II (AII), AII AT₂ type 2 receptor agonists, or analogs or fragments thereof.

5.6.1. Induction of Differentiation into Neurons

This example describes the induction of cord blood cells and/or embryonic-like stem cells to differentiate into neurons. The following protocol is employed to induce neuronal differentiation:

1. Placental stem cells are grown for 24 hr in preinduction media consisting of DMEM/20% FBS and 1 mM beta-mercaptoethanol.

2. Preinduction media is removed and cells are washed with PBS.

3. Neuronal induction media consisting of DMEM and 1-10 mM betamercaptoethanol is added. Alternatively, induction media consisting of DMEM/2% DMSO/200 μM butylated hydroxyanisole may be used to enhance neuronal differentiation efficiency.

4. In certain embodiments, morphologic and molecular changes may occur as early as 60 minutes after exposure to serum-free media and betamercaptoethanol (Woodbury et al., J. Neurosci. Res., 61:364-370). RT/PCR may be used to assess the expression of e.g., nerve growth factor receptor and neurofilament heavy chain genes.

5.6.2. Induction of Differentiation into Adipocytes

This example describes the induction of cord blood cells and/or embryonic-like stem cells to differentiate into adipocytes. The following protocol is employed to induce adipogenic differentiation:

1. Placental stem cells are grown in MSCGM (Bio Whittaker) or DMEM supplemented with 15% cord blood serum.

2. Three cycles of induction/maintenance are used. Each cycle consists of feeding the placental stem cells with Adipogenesis Induction Medium (Bio Whittaker) and culturing the cells for 3 days (at 37° C., 5% CO₂), followed by 1-3 days of culture in Adipogenesis Maintenance Medium (Bio Whittaker). An induction medium is used that contains 1 μM dexamethasone, 0.2 mM indomethacin, 0.01 mg/ml insulin, 0.5 mM IBMX, DMEM-high glucose, FBS, and antibiotics.

3. After 3 complete cycles of induction/maintenance, the cells are cultured for an additional 7 days in adipogenesis maintenance medium, replacing the medium every 2-3 days.

4. Adipogenesis may be assessed by the development of multiple intracytoplasmic lipid vesicles that can be easily observed using the lipophilic stain oil red 0. RT/PCR assays are employed to examine the expression of lipase and fatty acid binding protein genes.

5.6.3. Induction of Differentiation into Chondrocytes

This example describes the induction of cord blood cells and/or embryonic-like stem cells to differentiate into chondrocytes. The following protocol is employed to induce chondrogenic differentiation:

1. Placental stem cells are maintained in MSCGM (Bio Whittaker) or DMEM supplemented with 15% cord blood serum.

2. Placental stem cells are aliquoted into a sterile polypropylene tube. The cells are centrifuged (150×g for 5 minutes), and washed twice in Incomplete Chondrogenesis Medium (Bio Whittaker).

3. After the last wash, the cells are resuspended in Complete Chondrogenesis Medium (Bio Whittaker) containing 0.01 μg/ml TGF-beta-3 at a concentration of 5×10(5) cells/ml.

4. 0.5 ml of cells is aliquoted into a 15 ml polypropylene culture tube. The cells are pelleted at 150×g for 5 minutes. The pellet is left intact in the medium.

5. Loosely capped tubes are incubated at 37° C., 5% CO₂ for 24 hours.

6. The cell pellets are fed every 2-3 days with freshly prepared complete chondrogenesis medium.

7. Pellets are maintained suspended in medium by daily agitation using a low speed vortex.

8. Chondrogenic cell pellets are harvested after 14-28 days in culture.

9. Chondrogenesis may be characterized by e.g., observation of production of esoinophilic ground substance, assessing cell morphology, an/or RT/PCR for examining collagen 2 and collagen 9 gene expression.

5.6.4. Induction of Differentiation into Osteocytes

This example describes the induction of cord blood cells and/or embryonic-like stem cells to differentiate into osteocytes. The following protocol is employed to induce osteogenic differentiation:

1. Adherent cultures of placental stem cells are cultured in MSCGM (Bio Whittaker) or DMEM supplemented with 15% cord blood serum.

2. Cultures are rested for 24 hours in tissue culture flasks.

3. Osteogenic differentiation is induced by replacing MSCGM with Osteogenic Induction Medium (Bio Whittaker) containing 0.1 μM dexamethasone, 0.05 mM ascorbic acid-2-phosphate, 10 mM beta glycerophosphate.

4. Cells are fed every 3-4 days for 2-3 weeks with Osteogenic Induction Medium.

5. Differentiation is assayed using a calcium-specific stain and RT/PCR for alkaline phosphatase and osteopontin gene expression.

5.6.5. Induction of Differentiation into Hepatocytes

This example describes the induction of cord blood cells and/or embryonic-like stem cells to differentiate into hepatocytes. The following protocol is employed to induce hepatogenic differentiation:

1. Placental stem cells are cultured in DMEM/20% CBS supplemented with hepatocyte growth factor, 20 ng/ml; and epidermal growth factor, 100 ng/ml. KnockOut Serum Replacement may be used in lieu of FBS.

2. IL-6 50 ng/ml is added to induction flasks.

5.6.6. Induction of Differentiation into Pancreatic Cells

This example describes the induction of cord blood cells and/or embryonic-like stem cells to differentiate into pancreatic cells. The following protocol is employed to induce pancreatic differentiation:

1. Placental stem cells are cultured in DMEM/20% CBS, supplemented with basic fibroblast growth factor, 10 ng/ml; and transforming growth factor beta-1, 2 ng/ml. KnockOut Serum Replacement may be used in lieu of CBS.

2. Conditioned media from nestin-positive neuronal cell cultures is added to media at a 50/50 concentration.

3. Cells are cultured for 14-28 days, refeeding every 3-4 days.

4. Differentiation is characterized by assaying for insulin protein or insulin gene expression by RT/PCR.

5.6.7. Induction of Differentiation into Cardiac Cells

This example describes the induction of cord blood cells and/or embryonic-like stem cells to differentiate into cardiac cells. The following protocol is employed to induce myogenic differentiation:

1. Placental stem cells are cultured in DMEM/20% CBS, supplemented with retinoic acid, 1 μM; basic fibroblast growth factor, 10 ng/ml; and transforming growth factor beta-1, 2 ng/ml; and epidermal growth factor, 100 ng/ml. KnockOut Serum Replacement may be used in lieu of CBS.

2. Alternatively, placental stem cells are cultured in DMEM/20% CBS supplemented with 50 ng/ml Cardiotropin-1 for 24 hours.

3. Alternatively, placental stem cells are maintained in protein-free media for 5-7 days, then stimulated with human myocardium extract (escalating dose analysis). Myocardium extract is produced by homogenizing 1 gm human myocardium in 1% HEPES buffer supplemented with 1% cord blood serum. The suspension is incubated for 60 minutes, then centrifuged and the supernatant collected.

4. Cells are cultured for 10-14 days, refeeding every 3-4 days.

5. Differentiation is assessed using cardiac actin RT/PCR gene expression assays.

5.6.8. Characterization of Cord Blood Cells and/or Embryonic-like Stem Cells Prior to and/or After Differentiation

The embryonic-like stem cells, the cord blood cells and/or the populations of cord blood cells spiked with embryonic-like stem cells are characterized prior to and/or after differentiation by measuring changes in morphology and cell surface markers using techniques such as flow cytometry and immunocytochemistry, and measuring changes in gene expression using techniques, such as PCR. Cells that have been exposed to growth factors and/or that have differentiated are characterized by having the following cell surface markers: CD10, CD29, CD44, CD54, CD90, SH2, SH3, SH4, OCT-4 and ABC-p, or lacking the following cell surface markers: CD34, CD38, CD45, SSEA3 and SSEA4, or the equivalents thereof in different mammalian species. Preferably, the embryonic-like stem cell are characterized, prior to differentiation, by the presence of cell surface markers OCT-4 or ABC-p, and the absence of cell usrface markers CD34 and CD38. Stem cells bearing these markers are as versatile (e.g., pluripotent) as human embryonic stem cells. Cord blood cells are characterized, prior to differentiation, by the presence of cell surface markers CD34 and CD38. Differentiated cells derived from embryonic-like stem cells, cord blood cells and/or a populations of cord blood cells spiked with embryonic-like stem cells preferably do not express these markers.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All references cited herein are incorporated herein by reference in their entirety and for all purposes to the same extent as if each individual publication, patent or patent application was specifically and individually indicated to be incorporated by reference in its entirety for all purposes.

The citation of any publication is for its disclosure prior to the filing date and should not be construed as an admission that the present invention is not entitled to antedate such publication by virtue of prior invention.

What is claimed is:

1. A method of modulating the differentiation of a mammalian stem cell comprising contacting the stem cell with a compound that modulates JNK or MKK activity, under conditions suitable for differentiation of said stem cell.

2. The method of claim 1, wherein the compound inhibits JNK or MKK activity.

3. A method of conditioning a mammalian stem cell comprising contacting the stem cell with a compound that modulates JNK or MKK activity.

4. The method of claim 3, wherein the compound inhibits JNK or MKK activity.

5. The method of claim 3, wherein the mammalian stem cell or progenitor cell is cryopreserved and thawed prior to said conditioning.

6. A method of transplanting a mammalian stem cell or progenitor cell to a patient in need thereof comprising:
(a) contacting the stem cell or progenitor cell with a compound that inhibits JNK activity to produce a treated stem cell or progenitor cell; and
(b) transplanting the treated stem cell into said patient.

7. The method of claim 6, wherein step (b) comprises administering said treated stem cell with untreated cells.

8. The method of claim 7 wherein the untreated cell is selected from the group consisting of an embryonic stem cell, a placental stem cell, an adult stem cell, a cord blood cell, a bone marrow cell and a peripheral blood cell

9. The method of claim 7, wherein the mammalian stem cell has been cryopreserved and thawed prior to said contacting.

10. A method of producing a hematopoietic cell comprising contacting a mammalian stem cell with a compound that inhibits JNK or MKK activity under conditions suitable for differentiation of the stem cell, wherein said differentiation results in the production of a hematopoictic cell.

11. The method of claim 1, 3, 6 or 10 wherein the stem cell is selected from the group consisting of an embryonic stem cell, a placental stem cell, an adult stem cell, a cord blood cell, a peripheral blood cell, and a bone marrow cell.

12. The method of claim 1, 3, 6 or 10 wherein the stem cell is a human stem cell.

13. The method of claim 1, 3, 6 or 10 wherein the compound is an indazole, anilinopyrimidine, isothiazoloanthrone, isoxazoloanthrone, isoindolanthrone, or pyrazoloanthrone.

14. The method of claim 1, 3, 6 or 10 wherein the contacting step is conducted in vitro.

15. The method of claim 1, 3, 6 or 10 wherein the concentration of the compound is between 0.005 μg/ml and 5 mg/ml.

16. The method of claim 15, wherein the concentration of the compound is between 1 μg/ml and 2 mg/ml.

17. The method of claim 10 wherein said hematopoietic cell is a hematopoietic progenitor cell.

18. A pharmaceutical composition comprising a mammalian stem cell and a pharmaceutically-acceptable carrier, wherein said stem cell has been contacted with a compound that inhibits JNK or MKK activity for a time sufficient to cause modulation of differentiation or proliferation of said stem cell.

19. A pharmaceutical composition comprising a mammalian progenitor cell and a pharmaceutically-acceptable carrier, wherein said stem cell has been contacted with a compound that inhibits JNK or MKK activity for a time sufficient to cause modulation of differentiation or proliferation of said progenitor cell.

20. The pharmaceutical composition of claim 18 wherein the stem cell is selected from the group consisting of an embryonic stem cell, an adult cell, a cord blood cell, a placental stem cell or a peripheral blood stem cell.

21. The pharmaceutical composition of claim 18 or 19 wherein the compound is an imide or amide.

22. The pharmaceutical composition of claim 18 or 19 wherein the contacting step is conducted in cell culture.

23. The pharmaceutical composition of claim 18 or 19 wherein the concentration of the compound is from about 0.005 μg/ml to about 5 mg/ml.

24. The pharmaceutical composition of claim 18 or 19 wherein the concentration of the compound is from about 1 μg/ml to about 2 mg/ml.

25. The pharmaceutical composition of claim 18 wherein the stem cell is a human stem cell.

26. The pharmaceutical composition of claim 19, wherein said progenitor cell is a human progenitor cell.

27. The pharmaceutical composition of claim 19 wherein the progenitor cell is a hematopoietic progenitor cell.

28. The pharmaceutical composition of claim 18 or 19 wherein said differentiation is differentiation into a hematopoietic cell.

29. The pharmaceutical composition of claim 28 wherein the hematopoietic cell is CD34+ or CD38+.

30. The pharmaceutical composition of claim 28 wherein the hematopoietic cell is CD11b+.

31. A pharmaceutical composition comprising in a pharmaceutically acceptable carrier isolated cord blood cells and an isolated population of white blood cells, wherein the white blood cells are generated by a method comprising differentiating a stem cell under suitable conditions and in the presence of a compound that inhibits JNK activity or MKK activity, and isolating the white blood cells differentiated thereby.

32. A pharmaceutical composition comprising isolated cord blood cells and an isolated population of white blood cells, wherein the white blood cells are generated by a method comprising differentiating a stem cell under suitable conditions and in the presence of a compound that inhibits JNK activity or MKK activity, and isolating the white blood cells differentiated thereby.

33. The pharmaceutical composition of claim 31 or 32 wherein said differentiating is conducted in cell culture.

34. The pharmaceutical composition of claim 31 or 32 wherein the concentration of the compound is between 0.005 μg/ml and 5 mg/ml.

35. The pharmaceutical composition of claim 31 or 32 wherein the concentration of the compound is between 1 μg/ml and 2 mg/ml.

36. The pharmaceutical composition of claim 31 wherein the stem cell is a human stem cell.

37. The pharmaceutical composition of claim 32 wherein the progenitor cell is a hematopoietic progenitor cell.

38. A method of treating a mammalian subject in need of white blood cells comprising differentiating a stem cell or a progenitor cell under suitable conditions and in the presence of a compound that inhibits JNK or MKK activity, wherein said differentiating produces white blood cells, and administering a therapeutically effective amount of said white blood cells to said mammalian subject.

39. The method of claim 38 wherein the stem cell or progenitor cell is differentiated in vitro.

40. The method of claim 38 wherein the stem cell or progenitor cell is differentiated in a postpartum perfused placenta.

41. The method of claim 38 wherein the white blood cells are administered to the recipient mammalian subject in a cell preparation that is substantially free of red blood cells.

42. The method of claim 38 wherein the white blood cells are administered to the recipient mammalian subject in a cell preparation that comprises cord blood cells.

43. The method of claim 38 wherein the white blood cells are administered to the recipient mammalian subject in conjunction with a carrier.

44. The method of claim 38 wherein the white blood cells are administered intravenously.

45. The method of claim 38 wherein the white blood cells express incorporated genetic material of interest.

46. The method of claim 38 wherein said mammalian subject is human.

47. A method of transplanting bone marrow in a patient in need thereof, comprising transplanting to said patient cord blood, stem cells obtained from cord blood, peripheral blood or stem cells obtained from peripheral blood, wherein said cord blood, stem cells obtained from cord blood, peripheral blood or stem cells have been contacted with an inhibitor of JNK or MKK activity for a time sufficient to cause modulation of differentiation or proliferation of said stem cells.

48. The method of any of claims 1, 3, 6, 10, 38 or 47 wherein the JNK inhibitor or MKK inhibitor is a compound of the following structure (I): 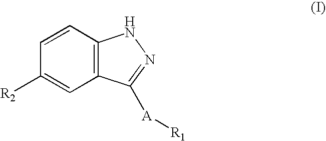
wherein:
A is a direct bond, —(CH₂)ₐ—, —(CH₂)_{b}CH═CH(CH₂)C—, or —(CH₂)_{b}C≡C(CH₂)_{c}—;
R₁ is aryl, heteroaryl or heterocycle fused to phenyl, each being optionally substituted with one to four substituents independently selected from R₃;
R₂ is —R₃, —R₄, —(CH₂)_{b}C(═O)R₅, —(CH₂)_{b}C(═O)OR₅, —(CH₂)_{b}C(═O)NR₅R₆, —(CH₂)_{b}C(═O)NR₅(CH₂)_{c}C(═O)R₆, —(CH₂)_{b}NR₅C(═O)R₆, —(CH₂)_{b}NR₅C(═O)NR₆R₇, —(CH₂)_{b}NR₅R₆, —(CH₂)_{b}OR₅, —(CH₂)_{b}SO_{d}R₅ or —(CH₂)_{b}SO₂NR₅R₆;
a is 1, 2, 3, 4, 5 or 6;
b and c are the same or different and at each occurrence independently selected from 0, 1, 2, 3 or 4;
d is at each occurrence 0, 1 or 2;
R₃ is at each occurrence independently halogen, hydroxy, carboxy, alkyl, alkoxy, haloalkyl, acyloxy, thioalkyl, sulfinylalkyl, sulfonylalkyl, hydroxyalkyl, aryl, arylalkyl, heterocycle, heterocycloalkyl, —C(═O)OR₈, —OC(═O)R₈, —C(═O)NR₈R₉, —C(═O)NR₈OR₉, —SO₂NR₈R₉, —NR₈SO₂R₉, —CN, —NO₂, —NR₈R₉, —NR₈C(═O)R₉, —NR₈C(═O)(CH₂)_{b}OR₉, —NR₈C(═O)(CH₂)_{b}R₉, —NR₈C(═O)(CH₂)_{b}NR₈R₉, —O(CH₂)_{b}NR₈R₉, or heterocycle fused to phenyl;
R₄ is alkyl, aryl, arylalkyl, heterocycle or heterocycloalkyl, each being optionally substituted with one to four substituents independently selected from R₃, or R₄ is halogen or hydroxy;
R₅, R₆ and R₇ are the same or different and at each occurrence independently hydrogen, alkyl, aryl, arylalkyl, heterocycle or heterocycloalkyl, wherein each of R₅, R₆ and R₇ are optionally substituted with one to four substituents independently selected from R₃; and
R₈ and R₉ are the same or different and at each occurrence independently hydrogen, alkyl, aryl, arylalkyl, heterocycle, or heterocycloalkyl, or R₈ and R₉ taken together with the atom or atoms to which they are bonded form a heterocycle, wherein each of R₈, R₉, and R₈ and R₉ taken together to form a heterocycle are optionally substituted with one to four substituents independently selected from R₃.

49. The method of any of claims 1, 3, 6, 10, 38 or 47 wherein the JNK inhibitor or MKK inhibitor is a compound of the following structure (II): 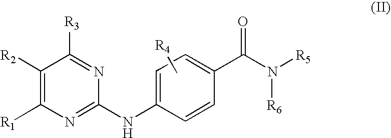
wherein:
R₁ is aryl or heteroaryl optionally substituted with one to four substituents independently selected from R₇;
R₂ is hydrogen;
R₃ is hydrogen or lower alkyl;
R₄ represents one to four optional substituents, wherein each substituent is the same or different and independently selected from halogen, hydroxy, lower alkyl and lower alkoxy;
R₅ and R₆ are the same or different and independently —R₈, —(CH₂)ₐC(═O)R₉, —(CH₂)ₐC(═O)OR₉, —(CH₂)ₐC(═O)NR₉R₁₀, —(CH₂)ₐC(═O)NR₉(CH₂)_{b}C(═O)R₁0, —(CH₂)ₐNR₉C(═O)R₁₀, (CH₂)ₐNR₁₁C(═O)NR₉R₁₀, —(CH₂)ₐNR₉R₁₀, —(CH₂)ₐOR₉, —(CH₂)ₐSO_{c}R₉ or —(CH₂)ₐSO₂NR₉R₁₀;
or R₅ and R₆ taken together with the nitrogen atom to which they are attached to form a heterocycle or substituted heterocycle;
R₇ is at each occurrence independently halogen, hydroxy, cyano, nitro, carboxy, alkyl, alkoxy, haloalkyl, acyloxy, thioalkyl, sulfinylalkyl, sulfonylalkyl, hydroxyalkyl, aryl, arylalkyl, heterocycle, substituted heterocycle, heterocycloalkyl, —C(═O)OR₈, —OC(═O)R₈, —C(═O)NR₈R₉, —C(═O)NR₈OR₉, —SO_{c}R₈, —SO_{c}NR₈R₉, —NR₈SO_{c}R₉, —NR₈R₉, —NR₈C(═O)R₉, —NR₈C(═O)(CH₂)_{b}OR₉, —NR₈C(═O)(CH₂)_{b}R₉, —O(CH₂)_{b}NR₈R₉, or heterocycle fused to phenyl;
R₈, R₉, R₁₀ and R₁₁ are the same or different and at each occurrence independently hydrogen, alkyl, aryl, arylalkyl, heterocycle, heterocycloalkyl;
or R₈ and R₉ taken together with the atom or atoms to which they are attached to form a heterocycle;
a and b are the same or different and at each occurrence independently selected from 0, 1, 2, 3 or 4; and
c is at each occurrence 0, 1 or 2.

50. The method of any of claims 1, 3, 6, 38 or 47 wherein the JNK inhibitor or MKK inhibitor is a compound of the following structure (III): 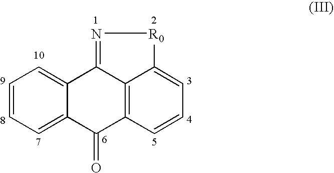
wherein R₀ is —O—, —S—, —S(O)—, —S(O)₂—, NH or —CH₂—;
the compound of structure (III) being: (i) unsubstituted, (ii) monosubstituted and having a first substituent, or (iii) disubstituted and having a first substituent and a second substituent;
the first or second substituent, when present, is at the 3, 4, 5, 7, 8, 9, or 10 position, wherein the first and second substituent, when present, are independently alkyl, hydroxy, halogen, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono-alkylaminoalkoxy, di-alkylaminoalkoxy, or a group represented by structure (a), (b), (c), (d), (e), or (f): 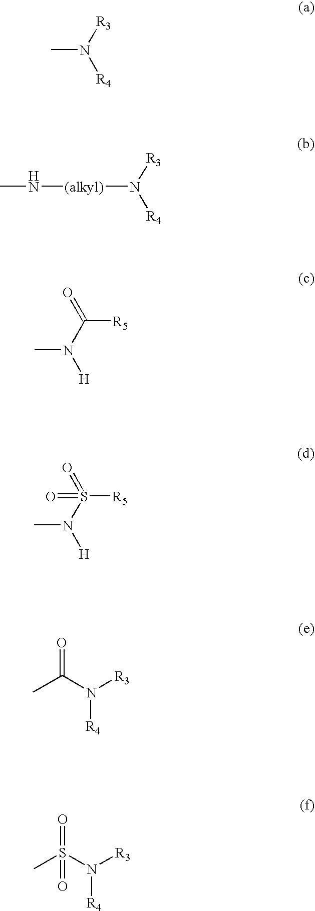
wherein R₃ and R₄ are taken together and represent alkylidene or a heteroatom-containing cyclic alkylidene or R₃ and R₄ are independently hydrogen, alkyl, cycloalkyl, aryl, arylalkyl, cycloalkylalkyl, aryloxyalkyl, alkoxyalkyl, aminoalkyl, mono-alkylaminoalkyl, or di-alkylaminoalkyl; and
R₅ is hydrogen, alkyl, cycloalkyl, aryl, arylalkyl, cycloalkylalkyl, alkoxy, alkoxyalkyl, alkoxycarbonylalkyl, amino, mono-alkylamino, di-alkylamino, arylamino, arylalkylamino, cycloalkylamino, cycloalkylalkylamino, aminoalkyl, mono-alkylaminoalkyl, or di-alkylaminoalkyl.

51. A method of treating or preventing a myeloproliferative disorder, comprising administering to a patient in need thereof an effective amount of a JNK inhibitor or an MKK inhibitor.

52. The method of claim 51, wherein the myeloproliferative disorder is polycythemia rubra vera; primary thrombocythemia; chronic myelogenous leukemia; acute or chronic granulocytic leukemia; acute or chronic myelomonocytic leukemia; myelofibro-erythroleukemia; or agnogenic myeloid metaplasia.

53. A method for treating or preventing a symptom of or an abnormality associated with a myeloproliferative disorder, comprising administering to a patient in need thereof an effective amount of a JNK inhibitor or an MKK inhibitor.

54. The method of claim 53, wherein the abnormality is clonal expansion of a multipotent hematopoietic progenitor cell with the overproduction of one or more of the formed elements of the blood, presence of Philadelphia chromosome or bcr-abl gene, teardrop poikilocytosis on peripheral blood smear, leukoerythroblastic blood picture, giant abnormal platelets, hypercellular bone marrow with reticular or collagen fibrosis or marked left-shifted myeloid series with a low percentage of promyelocytes and blasts.

55. A method for treating or preventing a myelodysplastic syndrome, comprising administering to a patient in need thereof an effective amount of a JNK inhibitor or an MKK inhibitor.

56. The method of claim 55, wherein the myelodysplastic syndrome is refractory anemia, refractory anemia with ringed sideroblasts, refractory anemia with excess blasts, refractory anemia with excess blasts in transformation, preleukemia or chronic myelomonocytic leukemia.

57. A method for treating or preventing a symptom of a myelodysplastic syndrome, comprising administering to a patient in need thereof an effective amount of a JNK inhibitor or an MKK inhibitor.

58. The method of claim 57, wherein the symptom is anemia, thrombocytopenia, neutropenia, bicytopenia or pancytopenia.
